SEQUENCE LISTING (5 sequences)

SEQ ID NO: 1 - skipped

SEQ ID NO: 2 - DNA, 20 bp, synthetic construct
  source 1..20: mol_type = other DNA
tctcaggattgaagacgata

SEQ ID NO: 3 - DNA, 20 bp, synthetic construct
  source 1..20: mol_type = other DNA
gagcacatattcgatggaga

SEQ ID NO: 4 - DNA, 21 bp, synthetic construct
  source 1..21: mol_type = other DNA
gcacagtgagcgtgagccggc

SEQ ID NO: 5 - DNA, 20 bp, synthetic construct
  source 1..20: mol_type = other DNA
ggcggcggagcaatggaccg

(19) United States
(12) Patent Application Publication
(10) Pub. No.: US 2025/0189527 A1
(43) Pub. Date: Jun. 12, 2025

(54) COMPOSITIONS AND METHODS FOR DETECTING, PREDICTING RISK OF DEVELOPING, AND TREATING LEPTOMENINGEAL DISEASE

(71) Applicant: Duke University, Durham, NC (US)

(72) Inventors: Dorothy SIPKINS, Durham, NC (US); Andrew WHITELEY, Durham, NC (US)

(21) Appl. No.: 18/844,550

(22) PCT Filed: Mar. 7, 2023
(86) PCT No.: PCT/US2023/063842

§ 371 Date: Sep. 6, 2024

Related U.S. Application Data
(60) Provisional application No. 63/317,210, filed on Mar. 7, 2022.

(51) Int. Cl. G01N 33/574

(52) U.S. Cl. CPC G01N 33/57415

(57) ABSTRACT

This disclosure provides compositions and methods for detecting, methods for predicting a risk of developing, and methods for treating systemic and brain parenchymal metastases including leptomeningeal disease (LMD). In particular, provided herein are methods for detecting and/or predicting a risk of developing systemic and brain parenchymal metastases including LMD in a subject (e.g., a human subject) through identifying the presence or absence of integrin a6 and/or breast cancer cells (BCCs) expressing integrin a6 in a sample obtained from the subject. In addition, provided herein are methods for treating, ameliorating, or preventing systemic and brain parenchymal metastases including LMD in a subject through inhibiting expression and/or activity of one or more of integrin a6, BCCs expressing integrin a6, neuroprotective factor glial cell line-derived neurotrophic factor (GDNF), and neural cell adhesion molecule (NCAM).

### STATEMENT REGARDING RELATED APPLICATIONS

This application claims priority to U.S. Provisional Patent Application No. 63/317,210, filed Mar. 7, 2022, the entire contents of which are incorporated herein by reference for all purposes.

### STATEMENT REGARDING FEDERAL FUNDING

This invention was made with Government support under Federal Grant no. R01 CA234580 awarded by the National Cancer Institute. The Federal Government has certain rights to this invention.

### SEQUENCE LISTING

The text of the computer readable sequence listing filed herewith, titled “41683-601_SequenceListing”, created Mar. 7, 2023, having a file size of 4,802 bytes, is hereby incorporated by reference in its entirety.

### FIELD OF THE INVENTION

This disclosure provides compositions and methods for detecting, methods for predicting a risk of developing, and methods for treating systemic and brain parenchymal metastases including leptomeningeal disease (LMD). In particular, provided herein are methods for detecting and/or predicting a risk of developing systemic and brain parenchymal metastases including LMD in a subject (e.g., a human subject) through identifying the presence or absence of integrin α6 and/or breast cancer cells (BCCs) expressing integrin α6 in a sample obtained from the subject. In addition, provided herein are methods for treating, ameliorating, or preventing systemic and brain parenchymal metastases including LMD in a subject through inhibiting expression and/or activity of one or more of integrin α6, BCCs expressing integrin α6, neuroprotective factor glial cell line-derived neurotrophic factor (GDNF), and neural cell adhesion molecule (NCAM).

### INTRODUCTION

Systemic and brain parenchymal metastases resulting from cancers such as breast cancer, are a devastating complication of metastatic cancer. One type of this metastasis included metastasis to the leptomeninges (LM), the membranes surrounding the brain and spinal cord. Of those breast cancer patients who develop brain metastasis, 10-20% will have leptomeningeal disease (LMD). Moreover, the incidence of LMD has been increasing as patients with metastatic disease experience prolonged survival in the era of novel targeted therapies. While there have been recent advances in treating brain parenchymal metastases, standard of care for LMD has remained essentially unchanged for decades. The current pillars of treatment are chemotherapy, radiation, and corticosteroids. Despite these interventions, most patients succumb to their disease within weeks-to-months of diagnosis. The paucity of targeted molecular therapies to treat LMD is attributed to our poor understanding of the molecular mechanisms governing leptomeningeal (LM) invasion. Identifying patients at risk for LMD and novel targeted therapies to prevent LM invasion could have significant impact on this population.

Improved techniques and methods for detecting, predicting a risk of developing, and treating systemic and brain parenchymal metastases including LMD are needed.

The present invention addresses these needs.

### SUMMARY OF THE INVENTION

Breast cancer cell (BCC) metastasis to the LM is a rapidly fatal disease complication. The paucity of targeted therapies to prevent or treat LM metastasis reflects a limited understanding of how BCCs invade and proliferate within this niche. Experiments conducted during the course of developing embodiments for the present invention demonstrated that BCCs in mice can invade the LM by abluminal migration along emissary vessels that connect vertebral/calvarial bone marrow and meninges, bypassing the restrictive blood-brain barrier (BBB). This process is dependent on BCC engagement with vascular basement membrane laminin through expression of the neuronal pathfinding molecule integrin α6. Once in the LM, BCCs remain in close contact with blood vessels and are encased by perivascular CSF1R+ meningeal macrophages. BCCs induce CSF1R+ cells to upregulate expression of the neuroprotective factor glial cell line-derived neurotrophic factor (GDNF), which is anti-apoptotic for BCCs. Specific ablation of CSFTR+ cells markedly decreases LM BCC growth, with LM disease progression rescued by recombinant GDNF. Taken together, these results indicate that BCCs co-opt neuronal pathfinding and survival mechanisms and resident macrophages to invade and thrive within the harsh LM microenvironment. Lastly, meningeal metastasis correlated with BCC α6 and stromal GDNF expression in a case-control series of bone-metastatic BC patients, indicating the clinical relevance of these pathways as therapeutic targets.

Accordingly, the present invention provides compositions and methods for detecting, methods for predicting a risk of developing, and methods for treating systemic and brain parenchymal metastases including LMD. In particular, provided herein are methods for detecting and/or predicting a risk of developing systemic and brain parenchymal metastases including LMD in a subject (e.g., a human subject) through identifying the presence or absence of integrin α6 and/or BCCs expressing integrin α6 in a sample obtained from the subject. In addition, provided herein are methods for treating, ameliorating, or preventing systemic and brain parenchymal metastases including LMD in a subject through inhibiting expression and/or activity of one or more of integrin α6, BCCs expressing integrin α6, GDNF, and NCAM.

In certain embodiments, the present invention provides a method for determining if a subject (e.g., a human subject suffering from breast cancer) is suffering from LMD comprising obtaining a biological sample from the subject, detecting the presence or absence of BCCs expressing integrin α6 in the biological sample, and determining that the subject is suffering from LMD if BCCs expressing integrin α6 are detected in the biological sample. In some embodiments, the biological sample is LM cells and/or LM tissue. In some embodiments, the biological sample is cerebrospinal fluid (CSF). In some embodiments, the biological sample is selected from a tissue sample, a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, a blood sample, a plasma sample, a serum sample, a whole blood sample, a secretion sample, an organ secretion sample, a cerebrospinal fluid (CSF) sample, a saliva sample, a urine sample, and/or a stool sample.

In certain embodiments, the present invention provides a method for determining if a subject (e.g., a human subject suffering from breast cancer) is suffering from LMD comprising obtaining a biological sample from the subject, detecting the presence or absence of integrin α6 in the biological sample, and determining that the subject is suffering from LMD if integrin α6 are detected in the biological sample. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides a method for determining if a subject (e.g., a human subject suffering from breast cancer) is suffering from LMD comprising obtaining a biological sample from the subject, detecting the presence or absence of NCAM in the biological sample, and determining that the subject is suffering from LMD if NCAM is detected in the biological sample. In some embodiments, detecting the presence or absence of NCAM in the biological sample comprises detecting the presence or absence of NCAM signaling. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides a method for determining if a subject (e.g., a human subject suffering from breast cancer) is suffering from LMD comprising obtaining a biological sample from the subject, detecting the presence or absence of GDNF in the biological sample, and determining that the subject is suffering from LMD if GDNF is detected in the biological sample. In some embodiments, detecting the presence or absence of GDNF in the biological sample comprises detecting the presence or absence of GDNF signaling. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides a method for determining if a subject (e.g., a human subject suffering from breast cancer) is suffering from LMD comprising obtaining a biological sample from the subject, detecting the presence or absence of one or more of GDNF, neuturin (NRTN), artemin (ARTN), and persephin (PSPN) in the biological sample, and determining that the subject is suffering from LMD if one or more of GDNF, NRTN, ARTN, and PSPN is detected in the biological sample. In some embodiments, detecting the presence or absence of one or more of GDNF comprises detecting the presence or absence of GDNF signaling. In some embodiments, detecting the presence or absence of one or more of NRTN comprises detecting the presence or absence of NRTN signaling. In some embodiments, detecting the presence or absence of one or more of ARTN comprises detecting the presence or absence of ARTN signaling. In some embodiments, detecting the presence or absence of one or more of PSPN comprises detecting the presence or absence of PSPN signaling. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides a method for determining if a subject (e.g., a human subject suffering from breast cancer) is suffering from LMD comprising obtaining a biological sample from the subject, detecting the presence or absence of one or more of: integrin α6, BCCs expressing integrin α6, NCAM, GDNF, NRTN, ARTN, and PSPN in the biological sample, and determining that the subject is suffering from LMD if of one or more of: integrin α6, BCCs expressing integrin α6, NCAM, GDNF, NRTN, ARTN, and PSPN are detected in the biological sample. In some embodiments, detecting the presence or absence of one or more of NCAM comprises detecting the presence or absence of NCAM signaling. In some embodiments, detecting the presence or absence of one or more of GDNF comprises detecting the presence or absence of GDNF signaling. In some embodiments, detecting the presence or absence of one or more of NRTN comprises detecting the presence or absence of NRTN signaling. In some embodiments, detecting the presence or absence of one or more of ARTN comprises detecting the presence or absence of ARTN signaling. In some embodiments, detecting the presence or absence of one or more of PSPN comprises detecting the presence or absence of PSPN signaling. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides a method for predicting a risk of developing LMD, the method comprising providing a biological sample from a subject (e.g., a human subject suffering from breast cancer), determining the level of BCCs expressing integrin α6 in the biological sample; and comparing the determined level to a reference level; wherein a higher level of BCCs expressing integrin α6 in the biological sample compared to the reference level is indicative of an increased risk of developing LMD; and wherein a level equal to or lower than the reference level is not indicative of an increased risk of developing LMD. In some embodiments, the reference level is an expression and/or activity level of BCCs expressing integrin α6 correlated with a low risk or no risk of developing LMD. In some embodiments, the biological sample is a tissue sample, a breast tissue sample, a breast tumor sample, a bone-marrow sample, a bone-marrow tumor sample, a brain tissue sample, a brain tumor sample, a LM tissue sample, a blood sample, a plasma sample, a serum sample, a whole blood sample, a secretion sample, an organ secretion sample, a CSF sample, a saliva sample, a urine sample, and/or a stool sample.

In certain embodiments, the present invention provides a method for predicting a risk of developing LMD, the method comprising providing a biological sample from a subject (e.g., a human subject suffering from breast cancer), determining the level of integrin α6 in the biological sample; and comparing the determined level to a reference level; wherein a higher level of integrin α6 in the biological sample compared to the reference level is indicative of an increased risk of developing LMD; and wherein a level equal to or lower than the reference level is not indicative of an increased risk of developing LMD. In some embodiments, the reference level is an expression and/or activity level of integrin α6 correlated with a low risk or no risk of developing LMD. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides a method for predicting a risk of developing LMD, the method comprising providing a biological from a subject (e.g., a human subject suffering from breast cancer), determining the level of NCAM in the biological sample; and comparing the determined level to a reference level; wherein a higher level of NCAM in the biological sample compared to the reference level is indicative of an increased risk of developing LMD; and wherein a level equal to or lower than the reference level is not indicative of an increased risk of developing LMD. In some embodiments, the reference level is an expression and/or activity level of NCAM correlated with a low risk or no risk of developing LMD. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides a method for predicting a risk of developing LMD, the method comprising providing a biological from a subject (e.g., a human subject suffering from breast cancer), determining the level of GDNF in the biological sample; and comparing the determined level to a reference level; wherein a higher level of GDNF in the biological sample compared to the reference level is indicative of an increased risk of developing LMD; and wherein a level equal to or lower than the reference level is not indicative of an increased risk of developing LMD. In some embodiments, the reference level is an expression and/or activity level of GDNF correlated with a low risk or no risk of developing LMD. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides a method for predicting a risk of developing LMD, the method comprising providing a biological from a subject (e.g., a human subject suffering from breast cancer), determining the level of one or more of GDNF, NRTN, ARTN, and PSPN in the biological sample; and comparing the determined level to a reference level; wherein a higher level of one or more of GDNF, NRTN, ARTN, and PSPN in the biological sample compared to the reference level is indicative of an increased risk of developing LMD; and wherein a level equal to or lower than the reference level is not indicative of an increased risk of developing LMD. In some embodiments, the reference level is an expression and/or activity level of one or more of GDNF, NRTN, ARTN, and PSPN correlated with a low risk or no risk of developing LMD. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides a method for predicting a risk of developing LMD, the method comprising providing a biological from a subject (e.g., a human subject suffering from breast cancer), determining the level of one or more of integrin α6, BCCs expressing integrin α6, GDNF, NRTN, ARTN, and PSPN in the biological sample; and comparing the determined level to a reference level; wherein a higher level of one or more of integrin α6, BCCs expressing integrin α6, GDNF, NRTN, ARTN, and PSPN in the biological sample compared to the reference level is indicative of an increased risk of developing LMD; and wherein a level equal to or lower than the reference level is not indicative of an increased risk of developing LMD. In some embodiments, the reference level is an expression and/or activity level of one or more of integrin α6, BCCs expressing integrin α6, GDNF, NRTN, ARTN, and PSPN correlated with a low risk or no risk of developing LMD. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides methods for characterizing a biological sample, comprising obtaining a biological sample from a subject (e.g., a human subject suffering from breast cancer), detecting the presence or absence of BCCs expressing integrin α6 in the biological sample, and characterizing the biological sample as consistent with LMD if BCCs expressing integrin α6 are detected in the biological sample. In some embodiments, the biological sample is a tissue sample, a breast tissue sample, a LM tissue sample, a blood sample, a plasma sample, a serum sample, a whole blood sample, a secretion sample, an organ secretion sample, a CSF sample, a saliva sample, a urine sample, and/or a stool sample. In some embodiments, the biological sample is LM cells and/or LM tissue. In some embodiments, the biological sample is CSF. In some embodiments, the biological sample is selected from a tissue sample, a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, a blood sample, a plasma sample, a serum sample, a whole blood sample, a secretion sample, an organ secretion sample, a cerebrospinal fluid (CSF) sample, a saliva sample, a urine sample, and/or a stool sample.

In certain embodiments, the present invention provides methods for characterizing a biological sample, comprising obtaining a biological sample from a subject (e.g., a human subject suffering from breast cancer), detecting the presence or absence of integrin α6 in the biological sample, and characterizing the biological sample as consistent with LMD if integrin α6 is detected in the biological sample. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides methods for characterizing a biological sample, comprising obtaining a biological sample from a subject (e.g., a human subject suffering from breast cancer), detecting the presence or absence of NCAM in the biological sample, and characterizing the biological sample as consistent with LMD if NCAM is detected in the biological sample. In some embodiments, detecting the presence or absence of NCAM in the biological sample comprises detecting the presence or absence of NCAM signaling in the biological sample. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides methods for characterizing a biological sample, comprising obtaining a biological sample from a subject (e.g., a human subject suffering from breast cancer), detecting the presence or absence of GDNF in the biological sample, and characterizing the biological sample as consistent with LMD if GDNF is detected in the biological sample. In some embodiments, detecting the presence or absence of GDNF in the biological sample comprises detecting the presence or absence of GDNF signaling in the biological sample. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides methods for characterizing a biological sample, comprising obtaining a biological sample from a subject (e.g., a human subject suffering from breast cancer), detecting the presence or absence of one or more of GDNF, NRTN, ARTN, and PSPN in the biological sample, and characterizing the biological sample as consistent with LMD if one or more of GDNF, NRTN, ARTN, and PSPN is detected in the biological sample. In some embodiments, detecting the presence or absence of GDNF in the biological sample comprises detecting the presence or absence of GDNF signaling in the biological sample. In some embodiments, detecting the presence or absence of NRTN in the biological sample comprises detecting the presence or absence of NRTN signaling in the biological sample. In some embodiments, detecting the presence or absence of ARTN in the biological sample comprises detecting the presence or absence of ARTN signaling in the biological sample. In some embodiments, detecting the presence or absence of PSPN in the biological sample comprises detecting the presence or absence of PSPN signaling in the biological sample. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides methods for characterizing a biological sample, comprising obtaining a biological sample from a subject (e.g., a human subject suffering from breast cancer), detecting the presence or absence of one or more of integrin α6, BCCs expressing integrin α6, NCAM, GDNF, NRTN, ARTN, and PSPN in the biological sample, and characterizing the biological sample as consistent with LMD if one or more of integrin α6, BCCs expressing integrin α6, NCAM, GDNF, NRTN, ARTN, and PSPN is detected in the biological sample. In some embodiments, detecting the presence or absence of NCAM in the biological sample comprises detecting the presence or absence of NCAM signaling in the biological sample. In some embodiments, detecting the presence or absence of GDNF in the biological sample comprises detecting the presence or absence of GDNF signaling in the biological sample. In some embodiments, detecting the presence or absence of NRTN in the biological sample comprises detecting the presence or absence of NRTN signaling in the biological sample. In some embodiments, detecting the presence or absence of ARTN in the biological sample comprises detecting the presence or absence of ARTN signaling in the biological sample. In some embodiments, detecting the presence or absence of PSPN in the biological sample comprises detecting the presence or absence of PSPN signaling in the biological sample. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides methods of treating, preventing, and/or ameliorating the symptoms of LMD in a subject in need thereof, the method comprising administering to the subject a therapeutically effective amount of an agent capable of one or more of the following: inhibiting expression of integrin α6, inhibiting activity of integrin α6, inhibiting BCC expression of integrin α6, inhibiting activity of BCCs expressing integrin α6, preventing metastasis of BCCs expressing integrin α6 to LM cells, preventing abluminal vascular LM invasion by BCCs expressing integrin α6, preventing BCCs expressing integrin α6 from entering the CNS along emissary vessels bridging bone marrow (BM) and LM, preventing survival of BCCs expressing integrin α6 in the LM, inhibiting LM related NCAM expression and/or activity, inhibiting LM related GDNF expression and/or activity, inhibiting LM related NRTN expression and/or activity, inhibiting LM related ARTN expression and/or activity, inhibiting LM related PSPN expression and/or activity, inhibiting LM related NCAM signaling, inhibiting LM related GDNF signaling, inhibiting LM related NRTN signaling, inhibiting LM related ARTN signaling, inhibiting LM related PSPN signaling, preventing engagement of BCCs having integrin α6 expression with vascular basement membrane laminin in LM cells, preventing encasement of BCC having integrin α6 expression in perivascular CSF1R+ meningeal macrophages, and preventing BCC having integrin α6 expression inducement of CSF1R+ cells to upregulate expression of the neuroprotective factor GDNF.

In certain embodiments, the present invention provides a method of treating LMD, preventing LMD, and/or ameliorating the symptoms of LMD in a subject, the method comprising:
(a) determining whether BCCs expressing integrin α6 are present or absent in a biological sample taken from the subject; and
(b) administering a therapeutically effective amount of an agent to the subject if BCCs expressing integrin α6 are present in the biological sample of LM cells, wherein the agent is capable of one or more of the following: inhibiting expression of integrin α6, inhibiting activity of integrin α6, inhibiting BCC expression of integrin α6, inhibiting activity of BCCs expressing integrin α6, preventing metastasis of BCCs expressing integrin α6 to LM cells, preventing abluminal vascular LM invasion by BCCs expressing integrin α6, preventing BCCs expressing integrin α6 from entering the CNS along emissary vessels bridging bone marrow (BM) and LM, preventing survival of BCCs expressing integrin α6 in the LM, inhibiting LM related NCAM expression and/or activity, inhibiting LM related GDNF expression and/or activity, inhibiting LM related NRTN expression and/or activity, inhibiting LM related ARTN expression and/or activity, inhibiting LM related PSPN expression and/or activity, inhibiting LM related NCAM signaling, inhibiting LM related GDNF signaling, inhibiting LM related NRTN signaling, inhibiting LM related ARTN signaling, inhibiting LM related PSPN signaling, preventing engagement of BCCs having integrin α6 expression with vascular basement membrane laminin in LM cells, preventing encasement of BCC having integrin α6 expression in perivascular CSF1R+ meningeal macrophages, and preventing BCC having integrin α6 expression inducement of CSF1R+ cells to upregulate expression of the neuroprotective factor GDNF. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample. In some embodiments, the biological sample is LM cells and/or LM tissue. In some embodiments, the biological sample is CSF. In some embodiments, the biological sample is selected from a tissue sample, a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, a blood sample, a plasma sample, a serum sample, a whole blood sample, a secretion sample, an organ secretion sample, a CSF sample, a saliva sample, a urine sample, and/or a stool sample.

In certain embodiments, the present invention provides a method of treating LMD, preventing LMD, and/or ameliorating the symptoms of LMD in a subject, the method comprising:
(a) determining the presence of absence of integrin α6 expression in a biological sample taken from the subject; and
(b) administering a therapeutically effective amount of an agent to the subject if integrin α6 expression in BCCs is identified, wherein the agent is capable of one or more of the following: inhibiting expression of integrin α6, inhibiting activity of integrin α6, inhibiting BCC expression of integrin α6, inhibiting activity of BCCs expressing integrin α6, preventing metastasis of BCCs expressing integrin α6 to LM cells, preventing abluminal vascular LM invasion by BCCs expressing integrin α6, preventing BCCs expressing integrin α6 from entering the CNS along emissary vessels bridging bone marrow (BM) and LM, preventing survival of BCCs expressing integrin α6 in the LM, inhibiting LM related NCAM expression and/or activity, inhibiting LM related GDNF expression and/or activity, inhibiting LM related NRTN expression and/or activity, inhibiting LM related ARTN expression and/or activity, inhibiting LM related PSPN expression and/or activity, inhibiting LM related NCAM signaling, inhibiting LM related GDNF signaling, inhibiting LM related NRTN signaling, inhibiting LM related ARTN signaling, inhibiting LM related PSPN signaling, preventing engagement of BCCs having integrin α6 expression with vascular basement membrane laminin in LM cells, preventing encasement of BCC having integrin α6 expression in perivascular CSF1R+ meningeal macrophages, and preventing BCC having integrin α6 expression inducement of CSF1R+ cells to upregulate expression of the neuroprotective factor GDNF.

In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides a method of treating LMD, preventing LMD, and/or ameliorating the symptoms of LMD in a subject, the method comprising:
(a) determining the presence of absence of one or more of integrin α6, BCCs expressing integrin α6, NCAM, GDNF, NRTN, ARTN, and PSPN in a biological sample taken from the subject; and
(b) administering a therapeutically effective amount of an agent to the subject if one or more of integrin α6, BCCs expressing integrin α6, NCAM, GDNF, NRTN, ARTN, and PSPN is identified, wherein the agent is capable of one or more of the following: inhibiting expression of integrin α6, inhibiting activity of integrin α6, inhibiting BCC expression of integrin α6, inhibiting activity of BCCs expressing integrin α6, preventing metastasis of BCCs expressing integrin α6 to LM cells, preventing abluminal vascular LM invasion by BCCs expressing integrin α6, preventing BCCs expressing integrin α6 from entering the CNS along emissary vessels bridging bone marrow (BM) and LM, preventing survival of BCCs expressing integrin α6 in the LM, inhibiting LM related NCAM expression and/or activity, inhibiting LM related GDNF expression and/or activity, inhibiting LM related NRTN expression and/or activity, inhibiting LM related ARTN expression and/or activity, inhibiting LM related PSPN expression and/or activity, inhibiting LM related NCAM signaling, inhibiting LM related GDNF signaling, inhibiting LM related NRTN signaling, inhibiting LM related ARTN signaling, inhibiting LM related PSPN signaling, preventing engagement of BCCs having integrin α6 expression with vascular basement membrane laminin in LM cells, preventing encasement of BCC having integrin α6 expression in perivascular CSF1R+ meningeal macrophages, and preventing BCC having integrin α6 expression inducement of CSF1R+ cells to upregulate expression of the neuroprotective factor GDNF.

In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

Such methods are not limited to particular type of LMD. In some embodiments, the LMD may be leptomeningeal, disseminated, and/or multicentric disease. In some instances, the LMD may be associated with one or more primary CNS tumors. In other cases, the LMD may be associated with one or more low-grade gliomas (LGGs). In some embodiments, the LMD is leptomeningeal carcinomatosis.

Such methods are not limited to particular symptoms of LMD. In some embodiments, the symptoms of LMD include, but are not limited to, headaches (usually associated with nausea, vomiting, light-headedness), gait difficulties from weakness or ataxia, memory problems, incontinence, and sensory abnormalities. In some embodiments, the symptoms of LMD include pain and seizures. CNS symptoms of LMD are generally divided into three anatomic groups (1) cerebral involvement including headache, lethargy, papilledema, behavioral changes, and gait disturbance; (2) cranial-nerve involvement including impaired vision, diplopia, hearing loss, and sensory deficits, including vertigo; and cranial-nerve palsies; and (3) spinal-root involvement including nuchal rigidity and neck and back pain, or invasion of the spinal roots.

Such methods are not limited to a particular type or kind of agent. In some embodiments, the agent is a small molecule, an antibody, nucleic acid molecule (e.g., siRNA, antisense oligonucleotide), a mimetic peptide, or a recombinant peptide.

In some embodiments, the agent may be comprised within any type or kind of composition. For example, in some embodiments, such a composition may be an over-the-counter composition, a pharmaceutical composition, or any kind of cosmetic composition.

Such methods are not limited to a particular manner of administration of the agent to the subject. In some embodiments, the administration is systemic administration. In some embodiments, the administration is local administration. In some embodiments, the administration is intravenous administration.

In some embodiments, the agent is a small molecule capable of inhibiting activity and/or expression of BCCs having integrin α6. In some embodiments, the agent is a small molecule capable of inhibiting activity and/or expression of integrin α6. In some embodiments, the agent is a small molecule capable of preventing expression of integrin α6 in BCCs. In some embodiments, the agent is a small molecule capable of preventing metastasis of BCCs expressing integrin α6 to LM cells.

Currently, there are no small molecule inhibitors that target integrin α6. While several monoclonal antibodies to target integrin α6 have been developed for use in biomedical research, they have not advanced to clinical trials. This is in part due to their affinity for both integrin heterodimers that contain integrin α6-integrin α6β1 and integrin α6β4. Integrin α6β4 is widely expressed by keratinocytes (skin cells), therefore targeting it can lead to skin blistering. In contrast, integrin α6β1 is only expressed by certain leukocytes and several reports indicate that its deletion does not severely impact hematopoiesis or hemostasis. Therefore, targeting integrin α6β1 and not α6β4 enables targeting of LMD while sparing keratinocytes. As such, in some embodiments, the agent is a small molecule capable of inhibiting activity and/or expression of BCCs expressing integrin α61. In some embodiments, the agent is a small molecule capable of inhibiting activity and/or expression of integrin α6β1.

In some embodiments, the agent is an antibody capable of inhibiting activity and/or expression of BCCs expressing integrin α6. In some embodiments, the agent is an antibody capable of inhibiting activity and/or expression of integrin α6. In some embodiments, the agent is an antibody capable of inhibiting activity and/or expression of BCCs expressing integrin α6β1. In some embodiments, the agent is an antibody capable of inhibiting activity and/or expression of integrin α6β1. In some embodiments, the agent is an antibody capable of preventing expression of integrin α6 in BCCs. In some embodiments, the agent is an antibody capable of preventing metastasis of BCCs expressing integrin α6 to LM cells.

Efforts to target the GDNF signaling and/or NCAM signaling in cancer have been limited. Indeed, small molecule inhibitors that block GDNF signaling and/or NCAM signaling are needed. The limited number of antibodies in clinical development have been designed to utilize tumor NCAM expression as a means to deliver conjugated drugs or induce antibody-dependent cytotoxic killing. Results using this approach have been disappointing. Monoclonal antibodies against GDNF have not been clinically pursued. Based on such needs, in some embodiments, the agent is a small molecule capable of inhibiting activity/expression of LM related GDNF signaling and/or NCAM signaling. In some embodiments, the agent is an antibody capable of inhibiting activity/expression of LM related GDNF signaling and/or NCAM signaling. In some embodiments, the agent is a small molecule capable of inhibiting activity/expression of LM related GDNF expression and/or activity. In some embodiments, the agent is a small molecule capable of inhibiting activity/expression of LM related NCAM expression and/or activity. In some embodiments, the agent is a small molecule capable of inhibiting activity/expression of LM related GDNF expression and/or activity. In some embodiments, the agent is a small molecule capable of inhibiting activity/expression of LM related NRTN expression and/or activity. In some embodiments, the agent is a small molecule capable of inhibiting activity/expression of LM related ARTN expression and/or activity. In some embodiments, the agent is a small molecule capable of inhibiting activity/expression of LM related PSPN expression and/or activity. In some embodiments, the agent is an antibody capable of inhibiting activity/expression of LM related NCAM expression and/or activity. In some embodiments, the agent is an antibody capable of inhibiting activity/expression of LM related GDNF expression and/or activity. In some embodiments, the agent is an antibody capable of inhibiting activity/expression of LM related NRTN expression and/or activity. In some embodiments, the agent is an antibody capable of inhibiting activity/expression of LM related ARTN expression and/or activity. In some embodiments, the agent is an antibody capable of inhibiting activity/expression of LM related PSPN expression and/or activity.

Such methods are not limited to a particular type of subject. In some embodiments, the subject is a mammalian subject. In some embodiments, the subject is a human patient. In some embodiments, the human patient is a human patient suffering from or having an increased risk for developing LMD. In some embodiments, the human patient is a human patient suffering from breast cancer. In some embodiments, the human patient is a human patient having BCCs expressing integrin α6.

In some embodiments, such methods for treating, preventing, and/or ameliorating the symptoms of LMD in a subject or subject further comprise co-administering to the subject an additional therapeutic agent.

In some embodiments, the additional therapeutic agent is administered to the subject simultaneously with the agent (e.g., an agent capable of inhibiting activity and/or expression of integrin α6) (e.g., an agent capable of inhibiting activity and/or expression of BCCs expressing integrin α6β1) (e.g., an agent capable of preventing expression of integrin α6 in BCCs) (e.g., an agent capable of preventing metastasis of BCCs expressing integrin α6 to LM cells) (e.g., an agent capable of preventing metastasis of BCCs expressing integrin α6β1 to LM cells) (e.g., an agent capable of inhibiting activity/expression of LM related GDNF signaling) (e.g., an agent capable of inhibiting activity/expression of LM related NCAM signaling) (e.g., an agent capable of inhibiting activity/expression of LM related GDNF) (e.g., an agent capable of inhibiting activity/expression of LM related NCAM) (e.g., an agent capable of inhibiting activity/expression of LM related NRTN) (e.g., an agent capable of inhibiting activity/expression of LM related ARTN) (e.g., an agent capable of inhibiting activity/expression of LM related PSPN) in the subject. In some embodiments, the additional therapeutic agent is administered to the subject prior to the agent in the subject. In some embodiments, the additional therapeutic agent is administered to the subject after the agent in the subject.

Such embodiments are not limited to a specific type or kind of additional therapeutic agent.

In some embodiments, the additional therapeutic agent is an anti-cancer therapy. In some embodiments, the anti-cancer therapy comprises radiation therapy and/or a chemotherapeutic agent. In some embodiments, the chemotherapeutic agent is one or more of the following: a taxane, a platinum-based agent, an anthracycline, an anthraquinone, an alkylating agent, a HER2 targeting therapy, vinorelbine, a nucleoside analog, ixabepilone, eribulin, cytarabine, a hormonal therapy, capecitabine, lapatinib, 5-FU, vincristine, etoposide, or methotrexate.

In some embodiments, the additional therapeutic agent is a palliative therapy. In some embodiments, the palliative therapy is an analgesic, an anticonvulsant, an antidepressant, an anxiolytic, a psychostimulant, modafinil, palliative radiation, corticosteroids, an H1 antagonist, a hematopoietic growth factor, and/or a blood transfusion.

Additional embodiments will be apparent to persons skilled in the relevant art based on the teachings contained herein.

### BRIEF DESCRIPTION OF DRAWINGS

FIG. 1A-M: BCCs invade the LM along emissary vessels. a, Schematic of leptomeninges (LM). b, Incidence of hindlimb paralysis (HLP) in 1833-P-engrafted mice. c, tdTomato (tdT) IHC image of spine section from a 1833-P-engrafted mouse. Dotted lines=LM. SA=subarachnoid space. d, Schematic of EO-LM2 derivation. e, HLP scores of EO-Parental, -LM1, or -LM2 mice at endpoint. Kruskal-Wallis with Dunn's multiple comparisons test. f, H&E of spine showing EO-LM2 cells entirely filling the meninges. Below dotted line=meninges. g, tdT IHC of the choroid plexus of EO-LM2 or 1833-P-tumored mice at post-engraftment day 0, 3, or clinical endpoint (EP). h, Intravital confocal microscopy (TVM) Z-stack images of the LM of mice at multiple time-points post-EO-LM2 engraftment. Arrowhead=perivascular tumor cluster. BM=bone marrow. i, Quantification of tdT+EO-LM2 cells detected in the brain parenchyma, choroid plexus, or LM by whole-mount confocal imaging, IHC, or IVM respectively (n=3-5 mice/group). One-sample t-test against zero for LM Day 3. j, Spine microCT of healthy vs. EO-LM2-tumored mice with HLP. Dotted lines outline spinal canal. Brackets=bone channels encasing emissary vessels. Bone channel measurements from microCT images: healthy control, n=3 mice/group, n=48-109 channels/mouse. One-way ANOVA with Tukey's post-hoc. k,l, H&E and tdT IHC showing EO-LM2 (k) and 1833-P (l) cells surrounding emissary vessels in the spine. Brackets=bone channels. Arrowheads=BCCs. Scale bars=50 μm. m, Immunofluorescence images of optically cleared skulls showing tumor cells (red) migrating on α-SMA+ emissary vessels (green) that passage through the BM (white) to the LM. Brackets=bone channels. Arrowheads=BCCs. *P<0.05, **P<0.01, ***P<0.001. ns=not significant.±s.e.m.

FIG. 2: MicroCT reveals intact vertebral/spinal canals in diseased mice. Representative axial microCT images of lumbosacral vertebrae from healthy mice or mice with HLP (EO-LM2 and 1833). Dotted lines outline spinal canals containing the spinal cord within the vertebral column. Scale bars=1 mm.

FIG. 3A-C: Diagram of hematogenous entry routes into the CNS and IVM of the CNS. a, Cartoon showing locations of the blood-brain and blood-CSF barriers in the central nervous system (CNS). 1. The choroid plexus (CP) is a secretory epithelium located within the ventricles of the brain that produces the cerebral spinal fluid (CSF). The epithelial cells of the CP form a continuous layer connected through tight junctions. It is vascularized by fenestrated vessels that, in combination with the CP epithelium, constitute the blood-CSF barrier. 2. Leptomeningeal blood vessels within the CSF-containing sub-arachnoid space have tight-junctions between endothelial cells that create a physical barrier between the blood and CSF. 3. Microvasculature within the brain parenchyma also contains tight-junctions and is surrounded by astrocytic endfeet forming the blood-brain barrier. b, Diagram of skull-thinning approach for intravital confocal microscopy (IVM) of the leptomeninges (LM). The image field of view (FOV) contains the LM, as well as a ring of intact calvarial bone marrow (BM) surrounding the thinned skull window. c, To scale diagram of the average size of the skull thinned imaging window (pink).

FIG. 4A-H: BCCs do not invade the LM through a hematogenous route. a, tdTomato (tdT) IHC of a brain from a mouse engrafted with 1833-P cells at clinical endpoint. Dotted lines=leptomeninges (LM). Scale=50 m. b, Representative intravital microscopy (IVM) confocal images of EG-LM2 cells in the calvarial bone marrow (BM) on days 0, 1, 2, and 3 post-engraftment. EG-LM2=red, BM vasculature=white. Scale=100 um. (n=3 mice per group). c, Frequency of BM and LM disease in mice on days 0, 1, 2, and 3 post-engraftment as determined by IVM. n=3 mice (day 0, 1, 2). n=4 mice (day 3) d, Representative IVM images of the BM and LM of 1833-P-tumored mice on days 0, 10, or 23 post-intracardiac engraftment. Dotted lines outline the skull thinned window revealing the LM vasculature. 1833-P=red, LM vasculature=white. Scale bar=100 um. (n=3 mice per group). e, Frequency of BM and LM disease in mice on days 0, 10, and 23 post-engraftment as determined by IVM. n=3 mice (day 0, 10). n=4 mice (day 23) f, Representative confocal whole mount images of the brain parenchyma of EO-LM2 or 1833-P-tumored mice at various time-points post-intracardiac engraftment. BCC=red. Brain microvessel=white. Arrows=extravasating BCCs. Scale=50 um. (n=3 mice per group). g, Quantification of 1833 cells detected in the brain parenchyma, choroid plexus, or LM by whole mount imaging, IHC, or IVM, respectively. (n=3-5 mice per group). s.e.m. h, Box and whisker plot showing the distance of brain parenchymal lesions from the pia mater on day 3 and 30 post-engraftment (1833) or day 3 and 15 post-engraftment (EO-LM2) as measured by fluorescence whole mount imaging of sagittal brain slices.

FIG. 5A-D: BCCs invade the LM along emissary vessels. a, Violin plot showing size distribution of bone channel diameters measured via microCT of vertebrae from individual healthy or tumored mice. n−3 mice/group, n=48-109 channels/mouse. Mean diameter above each violin plot. b, H&E showing EO-LM2 and 1833-cells surrounding emissary vessels in the spine. Brackets=bone channels. Arrowheads=BCCs. Scale bars=50 μm. c, Representative immunofluorescence (IF) images of optically cleared skulls harvested from mice on day 3 post-engraftment of EO-LM2 or EO-LM2-α6 KO cells. Osteosense (bone)=white. tdTomato (tumor)=red. α-SMA (vessel)=green. Arrowhead=tdT+ tumor cell. BM=bone marrow. LM=leptomeninges. d, Percentage of mice in which EO-LM2 or EO-LM2-α6 KO tumor cells were detected in α-SMA+ emissary vessel channels of the skull on day 3 post-engraftment as determined by IF imaging of the entire optically cleared skull. n=4 EO-LM2; n=3 EO-LM2-α6 KO. Fisher's Exact Test. *P=0.0286.

FIG. 6A-F: Validation of integrin α6 KO cell lines. a-c, Representative histograms of flow cytometry showing integrin α6 expression on 1833-P and EO-LM2 BCCs. Isotype controls=black. 1833-P and EO-LM2-P=red. 1833-tdT-α6 KO and EO-tdT-LM2-α6 KO=blue. d, Representative integrin α6 IHC on tissues of mice engrafted with wildtype (WT) or integrin α6 KO cells. Scale bars=50 μm. Dotted lines outline tumor metastases. e,f, In vitro proliferation of parental compared to α6 KO or NCAM KO BCCs. n=3 biological replicates. ±s.e.m. one-way ANOVA conducted on 72 h timepoints.

FIG. 7A-K: Integrin α6 promotes BCC metastasis to the LM. a, Invasion index (normalized to collagen I) of 1833-P vs. −α6KO cells through a laminin-enriched 3D matrix. n=3 biological replicates. Two-sided, unpaired Student's t-test. s.e.m. b, Kaplan-Meier survival curve for 1833-P vs. −α6KO mice. Two-sided log rank Mantel-Cox test, n 7 mice per group. c, Hindlimb paralysis (HLP) incidence for paired 1833-P vs. −α6KO mice euthanized when either reached a clinical endpoint. Parental n=8, α6KO n=7. Fisher's exact test. d, Representative tdT IHC of brain sections from mice in (c). Dotted lines=leptomeninges (LM). e, Quantification of timelapse migration assay of EO-LM2 vs. −α6KO cell displacement across a laminin-coated plate. n 794-928 tracks. Unpaired Student's t-test. ±s.e.m. f, Kaplan-Meier survival curve for EO-LM2 vs. −α6KO mice. Two-sided log rank Mantel-Cox test, EO-LM2 n−7, EO-LM2-α6KO n−8. g, HLP incidence for paired EO-LM2 vs. −α6KO mice euthanized when either reached a clinical endpoint. n=8 per group. Fisher's exact test. h, Representative tdT IHC of brain sections from mice in (g). Dotted lines=LM. i, Incidence of LMD in the brains and/or spines of MCF7-P vs. −α6 OE engrafted mice. Fisher's exact test. Representative tdT IHC of brain section. Dotted lines=LM. j, Representative intravital microscopy images and quantification of EO-LM2 vs. −α6KO cells (red) in the LM of mice on day 3 post-engraftment. k, Quantification of the number of cells in the LM from the mice in (j). n−3 mice per group. One-way ANOVA. ±s.e.m. All scale bars=50 m. *P<0.05, **P<0.01, ***P<0.001. ns=not significant. s.e.m.

FIG. 8A-N: Integrin α6 KO does not affect systemic BCC growth. a, Quantification of the number of tdTomato+(tdT) BCCs in the leptomeninges (LM) of mice engrafted with parental or α6 KO cells at matched time-points. n=10 brain sections quantified per mouse. n=6 mice/group. The difference in cell number between matched mouse pairs was taken and then log-transformed prior to running a One-sample t-test against zero. *P=0.0154, ***P=0.0003. b, Hindlimb paralysis (HLP) incidence for paired 1833-P vs. −α6 KO (alternative guide) mice euthanized when either reached a clinical endpoint. n=5. mice/group. Fisher's exact test. *P=0.0476. c, Mean abdominal tumor measurements of paired EO-LM2 or EO-LM2-α6 KO mice euthanized when either reached a clinical endpoint. Tumors pooled from n=4 mice/group. Two-way unpaired Students t-test. ns=not significant. f s.e.m. d,e Quantification of tumor burden by mean tdT signal/total tissue area of whole mount tissue slices of the lung (d) or the brain (e) imaged by confocal fluorescence microscopy. Tissue slices were collected from mice engrafted with EO-P, EO-LM2, or EO-LM2-α6 KO cells on day 15 post-engraftment. n=4-5 mice/group. One-way ANOVA with Tukey's post-hoc test. f, Representative low and high magnification calvarial bone marrow (BM) intravital microscopy (IVM) images from EO-LM2 or EO-LM2-α6 KO mice on day 0 (˜2 h post-engraftment). White=BM vessel. Red=tumor cell. Scale bar=250 m. g, Quantification of the total number of tdT+ tumor cells in the calvarial BM of mice from (f). n=3 mice/group. Two-sided Student's t-test. ns=not significant. h, Flow cytometry quantification of the percent of tdT+ tumor cells in BM of EO-P, EO-LM2, or EO-LM2-α6 KO mice on day 12 post-engraftment. One-way ANOVA with Tukey's post-hoc test. i, IVIS bioluminescent imaging of 1833-luciferase-P or 1833-luciferase-α6 KO mice on day 28 post-engraftment. n=6 mice per group. j, Quantification of IVIS imaging (photons/s) of non-CNS (lung and femur) regions of interest (ROIs) of mice in (i). Data log(10) transformed for presentation. Unpaired two-sided Student's t-test. ns not significant. ±s.e.m. k Quantification of tumor burden by mean tdT signal/total tissue area of whole mount tissue slices of the lung or brain imaged by confocal fluorescence microscopy. Tissue slices were collected from mice engrafted with 1833-P or 1833-α6 KO mice on day 25 post-engraftment. n=4-5 mice/group. Two-way ANOVA with Sidak's post-hoc test. ns=not significant. 1, Representative low and high magnification calvarial BM IVM images from 1833-P or 1833-α6 KO mice on day 0 (˜2 h post-engraftment). White=BM vessel. Red=tumor cell. Scale bar=250 μm. m, Quantification of the total number of tdT+ tumor cells in the calvarial BM of mice in (1). n=3 mice/group. Two-sided Student's t-test. ns=not significant. in, Flow cytometry quantification of the percent of tdT+ tumor cells in the BM of 1833-P or 1833-α6 KO mice on day 25 post-engraftment. Two-sided Student's t-test. ns=not significant.

FIG. 9A-G: Integrin α6 overexpression promotes LM metastasis but not peripheral metastasis in mice engrafted with the MCF7 cell line. a, Representative histogram of flow cytometry showing integrin α6 expression on MCF7-P and MCF7-α6 OE cells. b, Representative whole mount (lung, liver, brain) or intravital bone marrow (BM) confocal fluorescence images from MCF7-P vs. MCF7-α6 OE mice on day 14 post-engraftment. Quantification of lung, liver, and brain metastases tumor burden shown as mean tdT signal/total tissue area. Quantification of BM metastases shown as the total number of tdT+ tumor cells in the calvarial BM. Two-sided Student's t-test. ns=not significant. n=3-4 mice/group/±s.e.m. MCF7 cells=red. Vasculature=white. c, Incidence of hindlimb paralysis (HLP) at clinical endpoint in mice engrafted with the MCF7-P (n=5) or MCF7-α6 OE (n=6) cell line. d, Number of spine or brain sections containing tdT+ tumor cells in the leptomeninges (LM) of mice from (c). 10 brain and 14 spine sections per mouse. One-sample t-test against zero. *P=0.0395 (spine), *P=0.0221 (brain). s.e.m. e, Representative tdT IHC of the brains of endpoint MCF7-P or MCF7-α6 OE mice. Scale bar=50 μm. Dotted lines=LM. f, Number of brain sections containing tdT+ tumor cells in the choroid plexus of mice from (c). 10 brain sections per mouse. g, In vitro proliferation of MCF7-P vs. MCF7-α6 OE cells. Two-sided Student's t-test conducted at 72 hr. n=3 biological replicates. ±s.e.m.

FIG. 10A-B: The EO-LM2 and EO-P cell lines have equivalent integrin α6 expression. a, Representative histogram showing integrin α6 expression on EO-P and EO-LM2 BCCs via flow cytometry. Isotype=grey. EO-P=green. EO-LM2=magenta. b, Quantification of mean fluorescence intensity (MFI) of integrin α6 staining

FIG. 11A-Q: Meningeal macrophages promote BCC survival through GDNF/NCAM signaling. a, Volcano plot of differentially expressed genes in EO-LM2 vs. −P cells. b, Ncam1 mean fluorescence intensity (MFI) by flow cytometry. n=3 biological replicates. Two-sided, unpaired Student's t-test. c, Representative intravital microscopy (IVM) and quantification of EO-LM2 vs. −NCAM1KO cells in the LM of mice on day 3 post-engraftment. n=3 mice. Two-sided, unpaired Student's t-test. d, Hindlimb paralysis (HLP) incidence and representative brain section H&E of paired EO-LM2 vs. −NCAM1KO mice euthanized when either reached a clinical endpoint (EP). n=5 mice. Fisher's exact test. e, Percent live EO-LM2 or 1833-P cells after glucose deprivation in vitro+/−GDNF and +/−NCAM neutralizing antibody. n=3 biological replicates. One-way ANOVA with Tukey's multiple comparisons test. f, GDNF (red) immunofluorescence (IF) on meninges cytospins from healthy or EO-LM2-engrafted CSFIR-GFP mice. Dotted line=isotype. Arrows=CSFIR+ cells (green). n=3 mice; n=18-43 cells quantified/mouse. Two-way ANOVA with Tukey's multiple comparisons. g, Representative GDNF IHC on brain sections from healthy vs. 1833-P mice at EP. Arrowheads=GDNF. h, Representative CD206, GDNF, and tdT IF on brain section of 1833-P mouse at EP. Arrows=CD206+ macrophages. Arrowheads=tdT+1833 cells. i, Flow cytometry quantification of GDNF expression in cells isolated from meninges of EO-LM2 mice. n=3 biological replicates. One-way ANOVA. j, Representative 3D IVM image of EO-LM2 cells (red) co-localizing with CSF1R+ cells (green) surrounding LM vessels (white). k, Kaplan-Meier curves showing time to HLP score of 0.5 in veh. vs. PLX5622-treated EO-LM2 mice. Two-sided log rank Mantel-Cox test, n 9 control, n=10 PLX5622. 1, GDNF IF (magenta) on meninges cystopin from veh. or PLX5622-treated EO-LM2-engrafted CSF1R-GFP (green) mice at EP. n=4 mice; n=25-110 cells/mouse. Two-sided, unpaired Student's t-test. m, Representative 3D IVM images of EO-LM2 cells (red, arrowheads) co-localizing with CSF1R+ cells (green) surrounding LM vessels (white) in veh. vs. PLX5622-treated mice on day 3 or 10 post-engraftment. n, Quantification of EO-LM2 and CSF1R co-localizing (co-loc.) or non-co-localizing (no-coloc.) cells in the LM of mice in (m). n−3 mice. Two-way ANOVA with Tukey's multiple comparisons. o, Dosing strategy for intracerebroventricular (ICV) GDNF rescue experiment. p, Kaplan-Meier survival curve of time to HLP score of 0.5 of mice in (o). Two-sided log rank Mantel-Cox test n=9 veh. ICV, n=10 GDNF ICV. Scale bars for (f,l)=10 μm, all others=50 μm. *P<0.05, ***P<0.001, ****P<0.001. ns=not significant. s.e.m. q, data mining of The Cancer Genome Atlas revealed that breast cancer patients with high NCAM expression have worse overall survival compared to patients with low NCAM expression.

FIG. 12A-C: RNA-seq on EO-P vs. EO-LM2 cells. a, Principal component analysis of EO-P vs. EO-LM2 cells. n=3 biological replicates/group. b, Heat map showing the upregulation of neural genes and brain metastasis genes in the EO-LM2 cells. c, Table of the top 50 differentially expressed genes in the EO-LM2 cells compared to the EO-P cells.

FIG. 13A-E: EO-LM2, 1833, and MCF7 BCCs express NCAMs. a, Ncam1 mRNA fold-change in EO-LM2 vs. EO-P as assessed by RT-qPCR. n=3 biological replicates. Unpaired, two-sided Student's t-test. **P=0.0096. ±s.e.m. b, Representative NCAM1 western blot (WB) of whole cell lysates from EO-P, EO-LM2, and EO-LM2-NCAM1 KO cell lines cultured in vitro. Representative NCAM2 and integrin α6 WB of whole cell lysates from the MCF7-P, MCF7-α6 OE, and 1833-P cell lines cultured in vitro. c, Quantification of tumor burden by mean tdT signal/total tissue area of whole mount tissue slices of the lung imaged by confocal fluorescence microscopy. Tissue slices were collected from paired EO-LM2 or EO-LM2-NCAM1 KO mice euthanized when either reached clinical endpoint. n=5 mice/group. Two-sided Student's t-test. ns=not significant. ±s.e.m. d, Mean abdominal tumor diameter from mice in (c). Tumors pooled from n=5 mice per group. ±s.e.m. Unpaired Student's t-test. ns=not significant. e, HLP incidence for paired EO-LM2 and −NCAM1 KO (alternative clone) mice euthanized when either reached a clinical endpoint mice. n=6. Mice/group. Fisher's exact test. **P=0.0022.

FIG. 14A-C: Macrophages produce GDNF. a, Representative GDNF IHC on spine sections from healthy vs. tumored (EO-LM2) mice at endpoint. Arrowheads=GDNF. Scale bar=50 um. b, Representative CD206, GDNF, tdT immunofluorescence images of brain sections of tumored (1833-P) mice at endpoint. Arrows=CD206+ macrophages. Arrowheads=tdT+1833 cells. c, GDNF ELISA on conditioned media from EO-LM2 or 1833 cell, control media, or recombinant GDNF. Dotted lines represent the detection range of the ELISA.

FIG. 15A-H: Immune subset analysis of mice engrafted with the EO-tdt-LM2 cells versus healthy control. a, The proportion of monocytes, monocyte derived macrophages (MDMs), border associated macrophages (BAMs), neutrophils, T-cells, natural killer T-cells (NKT). natural killer (NK) cells, or B-cells out of total CD45+ cells in the meninges of EO-LM2-tumored or healthy mice. Meninges isolated on day 13 post-engraftment. n=3 samples per group. n=3 mice pooled per sample. s.e.m. b, The raw number of immune cell subsets in the meninges from mice in (a). Two-way ANOVA. s.e.m. c, The proportion of CD4+, CD8+, or CD4+FOXP3+ T-cells out of total CD3+ cells in the meninges of EO-LM2-tumored or healthy mice. Meninges isolated on day 13 post-engraftment. n=3 samples per group. n=3 mice pooled per sample. ±s.e.m. ns=not significant. d, The raw number of immune cell subsets in the meninges from mice in (c). Two-way ANOVA. ±s.e.m. ****P<0.0001. ns=not significant. e, The ratio of CD4+ over CD8+ T-cells from the mice in (c). ±s.e.m. Unpaired Student's t-test. ns=not significant. f, The proportion of effector memory, central memory, or naïve CD3+ T-cells from the mice in (c). Two-way ANOVA. ±s.e.m. ns=not significant. g, The proportion of progenitor exhausted, terminal exhausted, or PD1+CD44+ T-cells out of total CD8+FOXP3− T-cells from the mice in (c). s.e.m. Two-sided Student's t-test. ns=not significant. h, The number of CD45+ live cells in the meninges of EO-LM2-tumored or healthy mice. Meninges isolated on day 13 post-engraftment. n=3 samples per group. n=3 mice pooled per sample. ±s.e.m. Unpaired Student's t-test. *P=0.0287.

FIG. 16A-E: Meningeal macrophages co-localize with EO-LM2 cells and are depleted with the CSF1R inhibitor, PLX5622. a, Confocal intravital microscopy (IVM) of the brain leptomeninges (LM) showing tdTomato+EO-LM2 cells co-localizing with CSF1R+ myeloid cells in the perivascular space of LM vessels (white). Scale bar=50 m. b, Representative whole mount (brain parenchyma) and IVM (LM) images of CSF1R-GFP reporter mice treated with either control or PLX5622 (1200 mg kg⁻¹) formulated AIN-76A rodent chow for three days. CSF1R+ cells=green. Vessels=white. c, Quantification of the number of CSF1R+ cells per imaging field of view of mice in (b). Three 10× field of view images acquired per mouse. Unpaired, two-sided Student's t-test. n=3 mice per group. ***P=0.0001. ****P<0.0001. s.e.m. d, Dosing Strategy for experiment in FIG. 11k,l. e, Dosing strategy for experiment in FIG. 11m,n.

FIG. 17: Summary of clinical data for patients with meningeal-based or brain parenchymal metastases.

FIG. 18: Summary of clinical data for patients with primary breast tumors or bone marrow metastases.

FIG. 19A-G: Integrin α6 expression correlates with the incidence of LMD. a, Tumor integrin α6+ expression in biopsies of meningeal (n=7), brain parenchymal (n=23), and bone marrow (BM) (n=18) metastatic lesions and primary tumors (n=35) from breast cancer patients. Chi-squared test. ***P<0.001. b, Representative integrin α6 (brown) IHC images from (a). c,d, IHC on serial sections of a meningeal-based breast cancer metastasis from the patient series in (a). Single stain (c): GDNF (brown), CD68 (purple); Dual stain (d): GDNF (yellow), CD68 (purple). Dotted lines surround tumor nests. e,f, Machine learning (e) or manual (f) quantification of GDNF/CD68 dual stained slides. g, Cartoon showing integrin α6+ BCCs invading the LM along the laminin-rich abluminal surface of BM-LM bridging emissary vessels and then co-localizing with tumor-associated meningeal macrophages that secrete GDNF and activate NCAM-dependent tumor cell survival pathways.

FIG. 20A-B: Integrin α6 expression mediates survival of BC metastases in the LM niche. a. Number of tdT+ tumor cells enumerated in LM of brain histologic sections from intracerebroventricularly (icv) engrafted EO-LM2 vs. EO-LM2-α6KO mice. b. Representative tdT IHC of brain section from icv engrafted EO-LM2 mouse. Arrow points to breast cancer cells in the cerebrospinal fluid of the 3rd ventricle. Scale bar=100 μm.

### DEFINITIONS

As used herein, the term “administration” refers to the administration of a composition (e.g., a compound, a conjugate, or a preparation that includes a compound or conjugate as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and vitreal.

The term “effective amount” or “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “effective amount” does not in fact require successful treatment be achieved in a particular individual. Rather, an effective amount may be that amount that provides a particular desired pharmacological response in a significant number of subjects when administered to patients in need of such treatment. It is specifically understood that particular subjects may, in fact, be “refractory” to an “effective amount.” To give but one example, a refractory subject may have a low bioavailability such that clinical efficacy is not obtainable. In some embodiments, reference to an effective amount may be a reference to an amount as measured in one or more specific tissues (e.g., a tissue affected by the disease, disorder or condition) or fluids (e.g., blood, saliva, serum, sweat, tears, urine, etc). Those of ordinary skill in the art will appreciate that, in some embodiments, an effective amount may be formulated and/or administered in a single dose. In some embodiments, an effective amount may be formulated and/or administered in a plurality of doses, for example, as part of a dosing regimen.

A “palliative therapy,” as used herein refers to an therapy administered to a subject for the purpose of improving quality of life, e.g., by relieving one or more symptoms or side effects associated with a disease.

As used herein, the term “pharmaceutical composition” refers to an agent (e.g., an agent capable of inhibiting activity and/or expression of integrin α6) (e.g., an agent capable of inhibiting activity and/or expression of BCCs expressing integrin α6β1) (e.g., an agent capable of preventing expression of integrin α6 in BCCs) (e.g., an agent capable of preventing metastasis of BCCs expressing integrin α6 to LM cells) (e.g., an agent capable of preventing metastasis of BCCs expressing integrin α6β1 to LM cells) (e.g., an agent capable of inhibiting activity/expression of LM related GDNF signaling) (e.g., an agent capable of inhibiting activity/expression of LM related NCAM signaling) (e.g., an agent capable of inhibiting activity/expression of LM related GDNF) (e.g., an agent capable of inhibiting activity/expression of LM related NCAM) (e.g., an agent capable of inhibiting activity/expression of LM related NRTN) (e.g., an agent capable of inhibiting activity/expression of LM related ARTN) (e.g., an agent capable of inhibiting activity/expression of LM related PSPN), formulated together with one or more pharmaceutically acceptable carriers. In some embodiments, the agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

The term “subject,” as used herein, refers to a human or non-human animal (e.g., a mammal such as a non-human primate, horse, cow, or dog).

A “therapeutic regimen” refers to a dosing regimen whose administration across a relevant population is correlated with a desired or beneficial therapeutic outcome.

The term “treatment” (also “treat” or “treating”), in its broadest sense, refers to any administration of a substance (e.g., provided compositions) that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be administered to a subject who does not exhibit signs of the relevant disease, disorder and/or condition and/or of a subject who exhibits only early signs of the disease, disorder, and/or condition. Alternatively or additionally, in some embodiments, treatment may be administered to a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The terms “disease, disorder or physiological condition” as used herein includes, but is not limited to, any abnormal condition and/or disorder of a structure or a function that affects a part of an organism. It may be caused by an external factor, such as an infectious disease, or by internal dysfunctions, such as cancer, cancer metastasis, and the like.

As is known in the art, a cancer is generally considered as uncontrolled cell growth. The methods of the present invention can be used to treat any cancer, and any metastases thereof, including, but not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, pancreatic cancer, thyroid cancer, hepatic carcinoma, skin cancer, melanoma, brain cancer, neuroblastoma, myeloma, various types of head and neck cancer, acute lymphoblastic leukemia, acute myeloid leukemia, Ewing sarcoma and peripheral neuroepithelioma. In some embodiments, the cancer comprises metastatic cancer. In other embodiments, the cancer comprises metastatic breast cancer.

As used herein, the term “metastatic cancer” includes all forms of metastatic cancer, including systemic and brain parenchymal metastases, metastases to the leptomeninges (e.g., leptomeningeal disease), other meningeal diseases associated with metastatic cancer, and the like.

As used herein “leptomeningeal disease” (LMD) or “leptomeningeal cancer” or “leptomeningeal carcinomatosis” pertains to a rare complication of cancer in which the disease spreads to the membranes (meninges) surrounding the brain and spinal cord. LMD occurs in approximately 5% of people with cancer and is usually terminal. If left untreated, median survival is 4-6 weeks; if treated, median survival is 2-3 months. LMD may occur at any stage of cancer, either as a presenting sign or as a late complication, though it is associated frequently with relapse of cancer elsewhere in the body. LMD occurs with invasion to and subsequent proliferation of neoplastic cells in the subarachnoid space. Malignancies of diverse origins may spread to this space, which is bound by the leptomeninges. The leptomeninges consist of the arachnoid and the pia mater; the space between the two contains the CSF. When tumor cells enter the CSF (either by direct extension, as in primary brain tumors, or by hematogenous dissemination, as in leukemia), they are transported throughout the nervous system by CSF flow, causing either multifocal or diffuse infiltration of the leptomeninges in a sheetlike fashion along the surface of the brain and spinal cord.

As used herein, the term “biomarker” refers to a naturally occurring biological molecule present in a subject at varying concentrations useful in predicting the risk or incidence of a disease or a condition, such as metastatic cancer. For example, the biomarker can be a protein present in higher or lower amounts in a subject at risk for metastatic cancer. The biomarker can include nucleic acids, ribonucleic acids, or a polypeptide used as an indicator or marker for metastatic cancer in the subject. In some embodiments, the biomarker is a protein. A biomarker may also comprise any naturally or nonnaturally occurring polymorphism (e.g., single-nucleotide polymorphism [SNP]) present in a subject that is useful in predicting the risk or incidence of metastatic cancer. Suitable biomarkers include, but are not limited to, integrin α6, BCCs expressing integrin α6, GDNF, NCAM, GDNF/NCAM complex, macrophages and/or microglia, and combinations thereof, and the like. In some embodiments, the biomarker is one or more of integrin α6, BCCs expressing integrin α6, GDNF, NRTN, ARTN, and PSPN. In some embodiments, the biomarker is an expression and/or activity level of one or more of integrin α6, BCCs expressing integrin α6, GDNF, NRTN, ARTN, and PSPN.

### DETAILED DESCRIPTION OF THE INVENTION

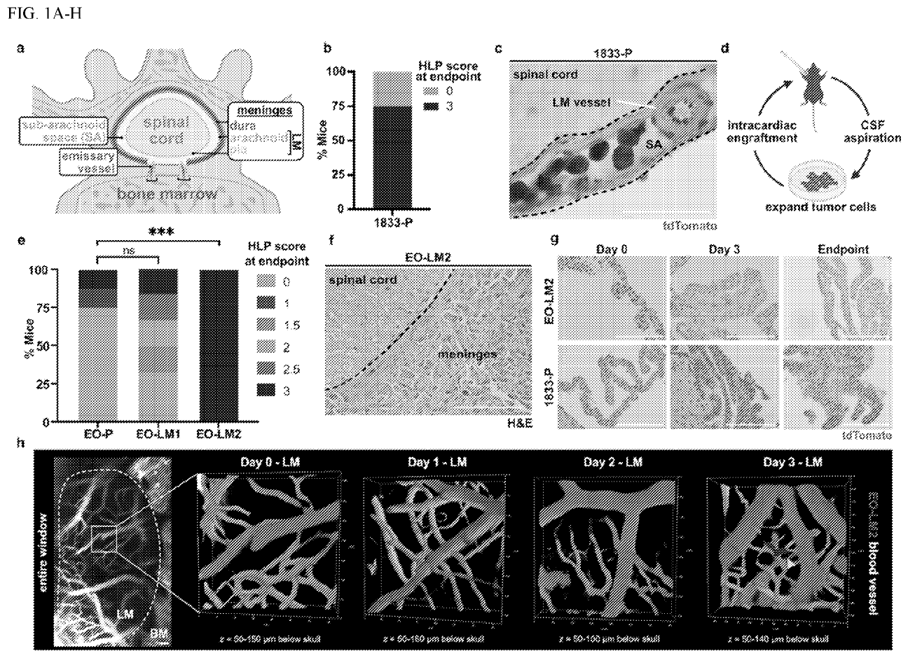

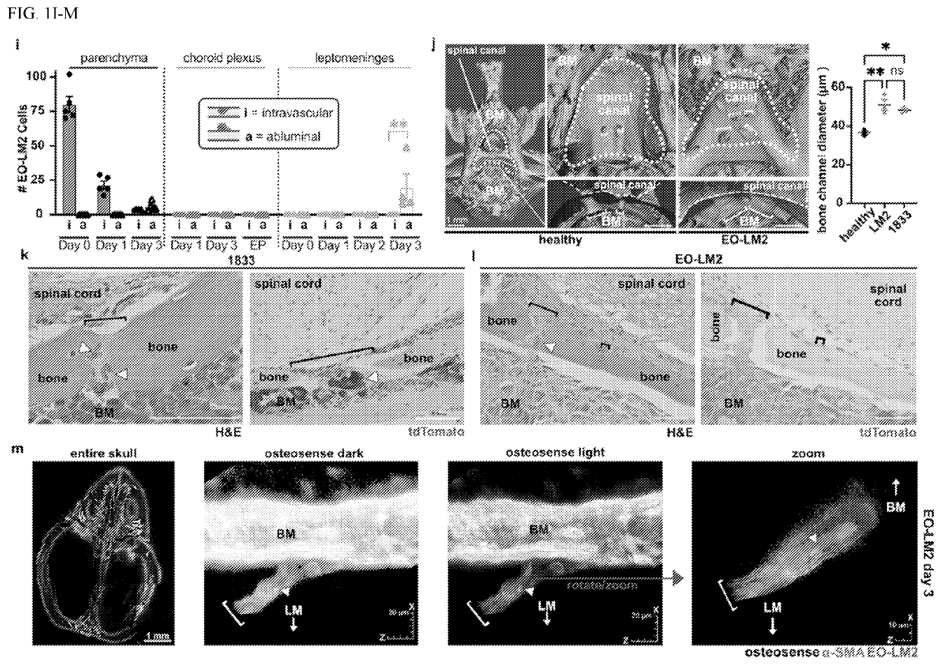

Breast cancer (BC) patients diagnosed with metastases to the leptomeninges (LM), the cerebrospinal fluid (CSF)-containing membranes surrounding the brain and spinal cord (FIG. 1a), have a median survival of less than six months^{1,2}. Despite recent advances in treating brain parenchymal metastases, there has been little improvement in leptomeningeal disease (LMD) outcomes³⁻⁵. A significant barrier to the development of effective, targeted therapies for LMD has been our limited understanding of the molecular mechanisms that regulate metastatic invasion and survival within the unique LM microenvironment, which is both anatomically and immunologically sequestered and nutrient-poor^{6,7}.

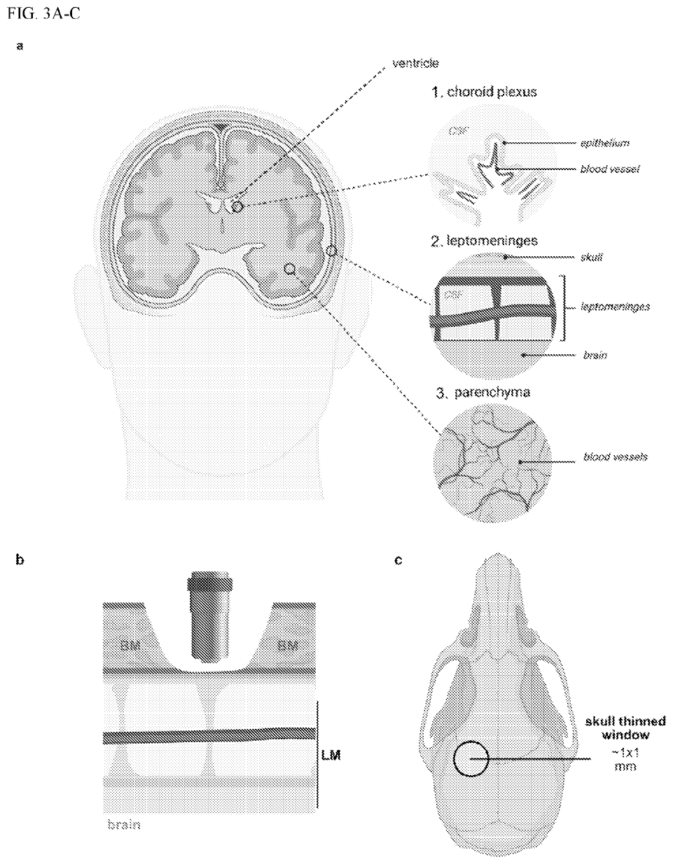

Elegant experiments have shown that BCCs can colonize the LM by compromising the blood-CSF barrier of the choroid plexus⁸—a CSF-producing secretory epithelium within the ventricles of the brain (FIG. 3a). However, many BC patients with LMD do not have evidence of choroid plexus involvement⁹. Experiments described herein investigated a potential alternative anatomic route for BC LM metastasis. Such experiments also probed the BCC crosstalk that enables tumor cells to subvert CNS-protective immunologic response mechanisms and thrive within the LM niche. Specifically, such experiments demonstrated that BCCs in mice can invade the LM by abluminal migration along emissary vessels that connect vertebral/calvarial bone marrow and meninges, bypassing the restrictive BBB. This process is dependent on BCC engagement with vascular basement membrane laminin through expression of the neuronal pathfinding molecule integrin α6. Once in the LM, BCCs remain in close contact with blood vessels and are encased by perivascular CSF1R+ meningeal macrophages. BCCs induce CSF1R+ cells to upregulate expression of the neuroprotective factor GDNF, which is anti-apoptotic for BCCs. Specific ablation of CSF1R+ cells markedly decreases LM BCC growth, with LM disease progression rescued by recombinant GDNF. Taken together, these results indicate that BCCs co-opt neuronal pathfinding and survival mechanisms and resident macrophages to invade and thrive within the harsh LM microenvironment. Lastly, it was shown that meningeal metastasis correlated with BCC α6 and stromal GDNF expression in a case-control series of bone-metastatic BC patients, indicating the clinical relevance of these pathways as therapeutic targets.

Accordingly, the present invention provides compositions and methods for detecting, methods for predicting a risk of developing, and methods for treating systemic and brain parenchymal metastases including LMD. In particular, provided herein are methods for detecting and/or predicting a risk of developing systemic and brain parenchymal metastases including LMD in a subject (e.g., a human subject) through identifying the presence or absence of integrin α6 and/or BCCs expressing integrin α6 in a sample obtained from the subject. In addition, provided herein are methods for treating, ameliorating, or preventing systemic and brain parenchymal metastases including LMD in a subject through inhibiting expression and/or activity of one or more of integrin α6, BCCs expressing integrin α6, GDNF, and NCAM.

In certain embodiments, the present invention provides a method for determining if a subject (e.g., a human subject suffering from breast cancer) is suffering from LMD comprising obtaining a biological sample from the subject, detecting the presence or absence of BCCs expressing integrin α6 in the biological sample, and determining that the subject is suffering from LMD if BCCs expressing integrin α6 are detected in the biological sample. In some embodiments, the biological sample is LM cells and/or LM tissue. In some embodiments, the biological sample is cerebrospinal fluid (CSF). In some embodiments, the biological sample is selected from a tissue sample, a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, a blood sample, a plasma sample, a serum sample, a whole blood sample, a secretion sample, an organ secretion sample, a cerebrospinal fluid (CSF) sample, a saliva sample, a urine sample, and/or a stool sample.

In certain embodiments, the present invention provides a method for determining if a subject (e.g., a human subject suffering from breast cancer) is suffering from LMD comprising obtaining a biological sample from the subject, detecting the presence or absence of integrin α6 in the biological sample, and determining that the subject is suffering from LMD if integrin α6 are detected in the biological sample. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides a method for determining if a subject (e.g., a human subject suffering from breast cancer) is suffering from LMD comprising obtaining a biological sample from the subject, detecting the presence or absence of NCAM in the biological sample, and determining that the subject is suffering from LMD if NCAM is detected in the biological sample. In some embodiments, detecting the presence or absence of NCAM in the biological sample comprises detecting the presence or absence of NCAM signaling. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides a method for determining if a subject (e.g., a human subject suffering from breast cancer) is suffering from LMD comprising obtaining a biological sample from the subject, detecting the presence or absence of GDNF in the biological sample, and determining that the subject is suffering from LMD if GDNF is detected in the biological sample. In some embodiments, detecting the presence or absence of GDNF in the biological sample comprises detecting the presence or absence of GDNF signaling. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides a method for determining if a subject (e.g., a human subject suffering from breast cancer) is suffering from LMD comprising obtaining a biological sample from the subject, detecting the presence or absence of one or more of GDNF, neuturin (NRTN), artemin (ARTN), and persephin (PSPN) in the biological sample, and determining that the subject is suffering from LMD if one or more of GDNF, NRTN, ARTN, and PSPN is detected in the biological sample. In some embodiments, detecting the presence or absence of one or more of GDNF comprises detecting the presence or absence of GDNF signaling. In some embodiments, detecting the presence or absence of one or more of NRTN comprises detecting the presence or absence of NRTN signaling. In some embodiments, detecting the presence or absence of one or more of ARTN comprises detecting the presence or absence of ARTN signaling. In some embodiments, detecting the presence or absence of one or more of PSPN comprises detecting the presence or absence of PSPN signaling. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides a method for determining if a subject (e.g., a human subject suffering from breast cancer) is suffering from LMD comprising obtaining a biological sample from the subject, detecting the presence or absence of one or more of: integrin α6, BCCs expressing integrin α6, NCAM, GDNF, NRTN, ARTN, and PSPN in the biological sample, and determining that the subject is suffering from LMD if of one or more of: integrin α6, BCCs expressing integrin α6, NCAM, GDNF, NRTN, ARTN, and PSPN are detected in the biological sample. In some embodiments, detecting the presence or absence of one or more of NCAM comprises detecting the presence or absence of NCAM signaling. In some embodiments, detecting the presence or absence of one or more of GDNF comprises detecting the presence or absence of GDNF signaling. In some embodiments, detecting the presence or absence of one or more of NRTN comprises detecting the presence or absence of NRTN signaling. In some embodiments, detecting the presence or absence of one or more of ARTN comprises detecting the presence or absence of ARTN signaling. In some embodiments, detecting the presence or absence of one or more of PSPN comprises detecting the presence or absence of PSPN signaling. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides a method for predicting a risk of developing LMD, the method comprising providing a biological sample from a subject (e.g., a human subject suffering from breast cancer), determining the level of BCCs expressing integrin α6 in the biological sample; and comparing the determined level to a reference level; wherein a higher level of BCCs expressing integrin α6 in the biological sample compared to the reference level is indicative of an increased risk of developing LMD; and wherein a level equal to or lower than the reference level is not indicative of an increased risk of developing LMD. In some embodiments, the reference level is an expression and/or activity level of BCCs expressing integrin α6 correlated with a low risk or no risk of developing LMD. In some embodiments, the biological sample is a tissue sample, a breast tissue sample, a breast tumor sample, a bone-marrow sample, a bone-marrow tumor sample, a brain tissue sample, a brain tumor sample, a LM tissue sample, a blood sample, a plasma sample, a serum sample, a whole blood sample, a secretion sample, an organ secretion sample, a CSF sample, a saliva sample, a urine sample, and/or a stool sample.

In certain embodiments, the present invention provides a method for predicting a risk of developing LMD, the method comprising providing a biological sample from a subject (e.g., a human subject suffering from breast cancer), determining the level of integrin α6 in the biological sample; and comparing the determined level to a reference level; wherein a higher level of integrin α6 in the biological sample compared to the reference level is indicative of an increased risk of developing LMD; and wherein a level equal to or lower than the reference level is not indicative of an increased risk of developing LMD. In some embodiments, the reference level is an expression and/or activity level of integrin α6 correlated with a low risk or no risk of developing LMD. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides a method for predicting a risk of developing LMD, the method comprising providing a biological from a subject (e.g., a human subject suffering from breast cancer), determining the level of NCAM in the biological sample; and comparing the determined level to a reference level; wherein a higher level of NCAM in the biological sample compared to the reference level is indicative of an increased risk of developing LMD; and wherein a level equal to or lower than the reference level is not indicative of an increased risk of developing LMD. In some embodiments, the reference level is an expression and/or activity level of NCAM correlated with a low risk or no risk of developing LMD. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides a method for predicting a risk of developing LMD, the method comprising providing a biological from a subject (e.g., a human subject suffering from breast cancer), determining the level of GDNF in the biological sample; and comparing the determined level to a reference level; wherein a higher level of GDNF in the biological sample compared to the reference level is indicative of an increased risk of developing LMD; and wherein a level equal to or lower than the reference level is not indicative of an increased risk of developing LMD. In some embodiments, the reference level is an expression and/or activity level of GDNF correlated with a low risk or no risk of developing LMD. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides a method for predicting a risk of developing LMD, the method comprising providing a biological from a subject (e.g., a human subject suffering from breast cancer), determining the level of one or more of GDNF, NRTN, ARTN, and PSPN in the biological sample; and comparing the determined level to a reference level; wherein a higher level of one or more of GDNF, NRTN, ARTN, and PSPN in the biological sample compared to the reference level is indicative of an increased risk of developing LMD; and wherein a level equal to or lower than the reference level is not indicative of an increased risk of developing LMD. In some embodiments, the reference level is an expression and/or activity level of one or more of GDNF, NRTN, ARTN, and PSPN correlated with a low risk or no risk of developing LMD. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides a method for predicting a risk of developing LMD, the method comprising providing a biological from a subject (e.g., a human subject suffering from breast cancer), determining the level of one or more of integrin α6, BCCs expressing integrin α6, GDNF, NRTN, ARTN, and PSPN in the biological sample; and comparing the determined level to a reference level; wherein a higher level of one or more of integrin α6, BCCs expressing integrin α6, GDNF, NRTN, ARTN, and PSPN in the biological sample compared to the reference level is indicative of an increased risk of developing LMD; and wherein a level equal to or lower than the reference level is not indicative of an increased risk of developing LMD. In some embodiments, the reference level is an expression and/or activity level of one or more of integrin α6, BCCs expressing integrin α6, GDNF, NRTN, ARTN, and PSPN correlated with a low risk or no risk of developing LMD. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides methods for characterizing a biological sample, comprising obtaining a biological sample from a subject (e.g., a human subject suffering from breast cancer), detecting the presence or absence of BCCs expressing integrin α6 in the biological sample, and characterizing the biological sample as consistent with LMD if BCCs expressing integrin α6 are detected in the biological sample. In some embodiments, the biological sample is a tissue sample, a breast tissue sample, a LM tissue sample, a blood sample, a plasma sample, a serum sample, a whole blood sample, a secretion sample, an organ secretion sample, a CSF sample, a saliva sample, a urine sample, and/or a stool sample. In some embodiments, the biological sample is LM cells and/or LM tissue. In some embodiments, the biological sample is CSF. In some embodiments, the biological sample is selected from a tissue sample, a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, a blood sample, a plasma sample, a serum sample, a whole blood sample, a secretion sample, an organ secretion sample, a cerebrospinal fluid (CSF) sample, a saliva sample, a urine sample, and/or a stool sample.

In certain embodiments, the present invention provides methods for characterizing a biological sample, comprising obtaining a biological sample from a subject (e.g., a human subject suffering from breast cancer), detecting the presence or absence of integrin α6 in the biological sample, and characterizing the biological sample as consistent with LMD if integrin α6 is detected in the biological sample. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides methods for characterizing a biological sample, comprising obtaining a biological sample from a subject (e.g., a human subject suffering from breast cancer), detecting the presence or absence of NCAM in the biological sample, and characterizing the biological sample as consistent with LMD if NCAM is detected in the biological sample. In some embodiments, detecting the presence or absence of NCAM in the biological sample comprises detecting the presence or absence of NCAM signaling in the biological sample. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides methods for characterizing a biological sample, comprising obtaining a biological sample from a subject (e.g., a human subject suffering from breast cancer), detecting the presence or absence of GDNF in the biological sample, and characterizing the biological sample as consistent with LMD if GDNF is detected in the biological sample. In some embodiments, detecting the presence or absence of GDNF in the biological sample comprises detecting the presence or absence of GDNF signaling in the biological sample. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides methods for characterizing a biological sample, comprising obtaining a biological sample from a subject (e.g., a human subject suffering from breast cancer), detecting the presence or absence of one or more of GDNF, NRTN, ARTN, and PSPN in the biological sample, and characterizing the biological sample as consistent with LMD if one or more of GDNF, NRTN, ARTN. and PSPN is detected in the biological sample. In some embodiments, detecting the presence or absence of GDNF in the biological sample comprises detecting the presence or absence of GDNF signaling in the biological sample. In some embodiments, detecting the presence or absence of NRTN in the biological sample comprises detecting the presence or absence of NRTN signaling in the biological sample. In some embodiments, detecting the presence or absence of ARTN in the biological sample comprises detecting the presence or absence of ARTN signaling in the biological sample. In some embodiments, detecting the presence or absence of PSPN in the biological sample comprises detecting the presence or absence of PSPN signaling in the biological sample. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides methods for characterizing a biological sample, comprising obtaining a biological sample from a subject (e.g., a human subject suffering from breast cancer), detecting the presence or absence of one or more of integrin α6, BCCs expressing integrin α6, NCAM, GDNF, NRTN, ARTN, and PSPN in the biological sample, and characterizing the biological sample as consistent with LMD if one or more of integrin α6, BCCs expressing integrin α6, NCAM, GDNF, NRTN, ARTN, and PSPN is detected in the biological sample. In some embodiments, detecting the presence or absence of NCAM in the biological sample comprises detecting the presence or absence of NCAM signaling in the biological sample. In some embodiments, detecting the presence or absence of GDNF in the biological sample comprises detecting the presence or absence of GDNF signaling in the biological sample. In some embodiments, detecting the presence or absence of NRTN in the biological sample comprises detecting the presence or absence of NRTN signaling in the biological sample. In some embodiments, detecting the presence or absence of ARTN in the biological sample comprises detecting the presence or absence of ARTN signaling in the biological sample. In some embodiments, detecting the presence or absence of PSPN in the biological sample comprises detecting the presence or absence of PSPN signaling in the biological sample. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides methods of treating, preventing, and/or ameliorating the symptoms of LMD in a subject in need thereof, the method comprising administering to the subject a therapeutically effective amount of an agent capable of one or more of the following: inhibiting expression of integrin α6, inhibiting activity of integrin α6, inhibiting BCC expression of integrin α6, inhibiting activity of BCCs expressing integrin α6, preventing metastasis of BCCs expressing integrin α6 to LM cells, preventing abluminal vascular LM invasion by BCCs expressing integrin α6, preventing BCCs expressing integrin α6 from entering the CNS along emissary vessels bridging bone marrow (BM) and LM, preventing survival of BCCs expressing integrin α6 in the LM, inhibiting LM related NCAM expression and/or activity, inhibiting LM related GDNF expression and/or activity, inhibiting LM related NRTN expression and/or activity, inhibiting LM related ARTN expression and/or activity, inhibiting LM related PSPN expression and/or activity, inhibiting LM related NCAM signaling, inhibiting LM related GDNF signaling, inhibiting LM related NRTN signaling, inhibiting LM related ARTN signaling, inhibiting LM related PSPN signaling, preventing engagement of BCCs having integrin α6 expression with vascular basement membrane laminin in LM cells, preventing encasement of BCC having integrin α6 expression in perivascular CSF1R+ meningeal macrophages, and preventing BCC having integrin α6 expression inducement of CSF1R+ cells to upregulate expression of the neuroprotective factor GDNF.

In certain embodiments, the present invention provides a method of treating LMD, preventing LMD, and/or ameliorating the symptoms of LMD in a subject, the method comprising:
(a) determining whether BCCs expressing integrin α6 are present or absent in a biological sample taken from the subject; and
(b) administering a therapeutically effective amount of an agent to the subject if BCCs expressing integrin α6 are present in the biological sample of LM cells, wherein the agent is capable of one or more of the following: inhibiting expression of integrin α6, inhibiting activity of integrin α6, inhibiting BCC expression of integrin α6, inhibiting activity of BCCs expressing integrin α6, preventing metastasis of BCCs expressing integrin α6 to LM cells, preventing abluminal vascular LM invasion by BCCs expressing integrin α6, preventing BCCs expressing integrin α6 from entering the CNS along emissary vessels bridging bone marrow (BM) and LM, preventing survival of BCCs expressing integrin α6 in the LM, inhibiting LM related NCAM expression and/or activity, inhibiting LM related GDNF expression and/or activity, inhibiting LM related NRTN expression and/or activity, inhibiting LM related ARTN expression and/or activity, inhibiting LM related PSPN expression and/or activity, inhibiting LM related NCAM signaling, inhibiting LM related GDNF signaling, inhibiting LM related NRTN signaling, inhibiting LM related ARTN signaling, inhibiting LM related PSPN signaling, preventing engagement of BCCs having integrin α6 expression with vascular basement membrane laminin in LM cells, preventing encasement of BCC having integrin α6 expression in perivascular CSF1R+ meningeal macrophages, and preventing BCC having integrin α6 expression inducement of CSFIR+ cells to upregulate expression of the neuroprotective factor GDNF. In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample. In some embodiments, the biological sample is LM cells and/or LM tissue. In some embodiments, the biological sample is CSF. In some embodiments, the biological sample is selected from a tissue sample, a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, a blood sample, a plasma sample, a serum sample, a whole blood sample, a secretion sample, an organ secretion sample, a CSF sample, a saliva sample, a urine sample, and/or a stool sample.

In certain embodiments, the present invention provides a method of treating LMD, preventing LMD, and/or ameliorating the symptoms of LMD in a subject, the method comprising:
(a) determining the presence of absence of integrin α6 expression in a biological sample taken from the subject; and
(b) administering a therapeutically effective amount of an agent to the subject if integrin α6 expression in BCCs is identified, wherein the agent is capable of one or more of the following: inhibiting expression of integrin α6, inhibiting activity of integrin α6, inhibiting BCC expression of integrin α6, inhibiting activity of BCCs expressing integrin α6, preventing metastasis of BCCs expressing integrin α6 to LM cells, preventing abluminal vascular LM invasion by BCCs expressing integrin α6, preventing BCCs expressing integrin α6 from entering the CNS along emissary vessels bridging bone marrow (BM) and LM, preventing survival of BCCs expressing integrin α6 in the LM, inhibiting LM related NCAM expression and/or activity, inhibiting LM related GDNF expression and/or activity, inhibiting LM related NRTN expression and/or activity, inhibiting LM related ARTN expression and/or activity, inhibiting LM related PSPN expression and/or activity, inhibiting LM related NCAM signaling, inhibiting LM related GDNF signaling, inhibiting LM related NRTN signaling, inhibiting LM related ARTN signaling, inhibiting LM related PSPN signaling, preventing engagement of BCCs having integrin α6 expression with vascular basement membrane laminin in LM cells, preventing encasement of BCC having integrin α6 expression in perivascular CSF1R+ meningeal macrophages, and preventing BCC having integrin α6 expression inducement of CSF1R+ cells to upregulate expression of the neuroprotective factor GDNF.

In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

In certain embodiments, the present invention provides a method of treating LMD, preventing LMD, and/or ameliorating the symptoms of LMD in a subject, the method comprising.
(a) determining the presence of absence of one or more of integrin α6, BCCs expressing integrin α6, NCAM, GDNF, NRTN, ARTN, and PSPN in a biological sample taken from the subject; and
(b) administering a therapeutically effective amount of an agent to the subject if one or more of integrin α6, BCCs expressing integrin α6, NCAM, GDNF, NRTN, ARTN, and PSPN is identified, wherein the agent is capable of one or more of the following: inhibiting expression of integrin α6, inhibiting activity of integrin α6, inhibiting BCC expression of integrin α6, inhibiting activity of BCCs expressing integrin α6, preventing metastasis of BCCs expressing integrin α6 to LM cells, preventing abluminal vascular LM invasion by BCCs expressing integrin α6, preventing BCCs expressing integrin α6 from entering the CNS along emissary vessels bridging bone marrow (BM) and LM, preventing survival of BCCs expressing integrin α6 in the LM, inhibiting LM related NCAM expression and/or activity, inhibiting LM related GDNF expression and/or activity, inhibiting LM related NRTN expression and/or activity, inhibiting LM related ARTN expression and/or activity, inhibiting LM related PSPN expression and/or activity, inhibiting LM related NCAM signaling, inhibiting LM related GDNF signaling, inhibiting LM related NRTN signaling, inhibiting LM related ARTN signaling, inhibiting LM related PSPN signaling, preventing engagement of BCCs having integrin α6 expression with vascular basement membrane laminin in LM cells, preventing encasement of BCC having integrin α6 expression in perivascular CSF1R+ meningeal macrophages, and preventing BCC having integrin α6 expression inducement of CSF1R+ cells to upregulate expression of the neuroprotective factor GDNF.

In some embodiments, the biological sample is LM cells and/or LM tissue. In some embodiments, the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

Such methods are not limited to particular type of LMD. In some embodiments, the LMD may be leptomeningeal, disseminated, and/or multicentric disease. In some instances, the LMD may be associated with one or more primary CNS tumors. In other cases, the LMD may be associated with one or more low-grade gliomas (LGGs). In some embodiments, the LMD is leptomeningeal carcinomatosis.

Such methods are not limited to particular symptoms of LMD. In some embodiments, the symptoms of LMD include, but are not limited to, headaches (usually associated with nausea, vomiting, light-headedness), gait difficulties from weakness or ataxia, memory problems, incontinence, and sensory abnormalities. In some embodiments, the symptoms of LMD include pain and seizures. CNS symptoms of LMD are generally divided into three anatomic groups (1) cerebral involvement including headache, lethargy, papilledema, behavioral changes, and gait disturbance; (2) cranial-nerve involvement including impaired vision, diplopia, hearing loss, and sensory deficits, including vertigo; and cranial-nerve palsies; and (3) spinal-root involvement including nuchal rigidity and neck and back pain, or invasion of the spinal roots.

Such methods are not limited to a particular type or kind of agent. In some embodiments, the agent is a small molecule, an antibody, nucleic acid molecule (e.g., siRNA, antisense oligonucleotide), a mimetic peptide, or a recombinant peptide.

In some embodiments, the agent may be comprised within any type or kind of composition. For example, in some embodiments, such a composition may be an over-the-counter composition, a pharmaceutical composition, or any kind of cosmetic composition.

Such methods are not limited to a particular manner of administration of the agent to the subject. In some embodiments, the administration is systemic administration. In some embodiments, the administration is local administration. In some embodiments, the administration is intravenous administration.

In some embodiments, the agent is a small molecule capable of inhibiting activity and/or expression of BCCs having integrin α6. In some embodiments, the agent is a small molecule capable of inhibiting activity and/or expression of integrin α6. In some embodiments, the agent is a small molecule capable of preventing expression of integrin α6 in BCCs. In some embodiments, the agent is a small molecule capable of preventing metastasis of BCCs expressing integrin α6 to LM cells.

Currently, there are no small molecule inhibitors that target integrin α6. While several monoclonal antibodies to target integrin α6 have been developed for use in biomedical research, they have not advanced to clinical trials. This is in part due to their affinity for both integrin heterodimers that contain integrin α6-integrin α6β1 and integrin α6β4. Integrin α6β4 is widely expressed by keratinocytes (skin cells), therefore targeting it can lead to skin blistering. In contrast, integrin α6β1 is only expressed by certain leukocytes and several reports indicate that its deletion does not severely impact hematopoiesis or hemostasis. Therefore, targeting integrin α6β1 and not α6β4 enables targeting of LMD while sparing keratinocytes. As such, in some embodiments, the agent is a small molecule capable of inhibiting activity and/or expression of BCCs expressing integrin α6β1. In some embodiments, the agent is a small molecule capable of inhibiting activity and/or expression of integrin α6β1.

In some embodiments, the agent is an antibody capable of inhibiting activity and/or expression of BCCs expressing integrin α6. In some embodiments, the agent is an antibody capable of inhibiting activity and/or expression of integrin α6. In some embodiments, the agent is an antibody capable of inhibiting activity and/or expression of BCCs expressing integrin α6β1. In some embodiments, the agent is an antibody capable of inhibiting activity and/or expression of integrin α6β1. In some embodiments, the agent is an antibody capable of preventing expression of integrin α6 in BCCs. In some embodiments, the agent is an antibody capable of preventing metastasis of BCCs expressing integrin α6 to LM cells.

Efforts to target the GDNF signaling and/or NCAM signaling in cancer have been limited. Indeed, small molecule inhibitors that block GDNF signaling and/or NCAM signaling are needed. The limited number of antibodies in clinical development have been designed to utilize tumor NCAM expression as a means to deliver conjugated drugs or induce antibody-dependent cytotoxic killing. Results using this approach have been disappointing. Monoclonal antibodies against GDNF have not been clinically pursued. Based on such needs, in some embodiments, the agent is a small molecule capable of inhibiting activity/expression of LM related GDNF signaling and/or NCAM signaling. In some embodiments, the agent is an antibody capable of inhibiting activity/expression of LM related GDNF signaling and/or NCAM signaling. In some embodiments, the agent is a small molecule capable of inhibiting activity/expression of LM related GDNF expression and/or activity. In some embodiments, the agent is a small molecule capable of inhibiting activity/expression of LM related NCAM expression and/or activity. In some embodiments, the agent is a small molecule capable of inhibiting activity/expression of LM related GDNF expression and/or activity. In some embodiments, the agent is a small molecule capable of inhibiting activity/expression of LM related NRTN expression and/or activity. In some embodiments, the agent is a small molecule capable of inhibiting activity/expression of LM related ARTN expression and/or activity. In some embodiments, the agent is a small molecule capable of inhibiting activity/expression of LM related PSPN expression and/or activity. In some embodiments, the agent is an antibody capable of inhibiting activity/expression of LM related NCAM expression and/or activity. In some embodiments, the agent is an antibody capable of inhibiting activity/expression of LM related GDNF expression and/or activity. In some embodiments, the agent is an antibody capable of inhibiting activity/expression of LM related NRTN expression and/or activity. In some embodiments, the agent is an antibody capable of inhibiting activity/expression of LM related ARTN expression and/or activity. In some embodiments, the agent is an antibody capable of inhibiting activity/expression of LM related PSPN expression and/or activity.

Such methods are not limited to a particular type of subject. In some embodiments, the subject is a mammalian subject. In some embodiments, the subject is a human patient. In some embodiments, the human patient is a human patient suffering from or having an increased risk for developing LMD. In some embodiments, the human patient is a human patient suffering from breast cancer. In some embodiments, the human patient is a human patient having BCCs expressing integrin α6.

In some embodiments, such methods for treating, preventing, and/or ameliorating the symptoms of LMD in a subject or subject further comprise co-administering to the subject an additional therapeutic agent.

In some embodiments, the additional therapeutic agent is administered to the subject simultaneously with the agent (e.g., an agent capable of inhibiting activity and/or expression of integrin α6) (e.g., an agent capable of inhibiting activity and/or expression of BCCs expressing integrin α6β1) (e.g., an agent capable of preventing expression of integrin α6 in BCCs) (e.g., an agent capable of preventing metastasis of BCCs expressing integrin α6 to LM cells) (e.g., an agent capable of preventing metastasis of BCCs expressing integrin α6β1 to LM cells) (e.g., an agent capable of inhibiting activity/expression of LM related GDNF signaling) (e.g., an agent capable of inhibiting activity/expression of LM related NCAM signaling) (e.g., an agent capable of inhibiting activity/expression of LM related GDNF) (e.g., an agent capable of inhibiting activity/expression of LM related NCAM) (e.g., an agent capable of inhibiting activity/expression of LM related NRTN) (e.g., an agent capable of inhibiting activity/expression of LM related ARTN) (e.g., an agent capable of inhibiting activity/expression of LM related PSPN) in the subject. In some embodiments, the additional therapeutic agent is administered to the subject prior to the agent in the subject. In some embodiments, the additional therapeutic agent is administered to the subject after the agent in the subject.

Such embodiments are not limited to a specific type or kind of additional therapeutic agent.

In some embodiments, the additional therapeutic agent is a palliative therapy. In some embodiments, the palliative therapy is an analgesic, an anticonvulsant, an antidepressant, an anxiolytic, a psychostimulant, modafinil, palliative radiation, corticosteroids, an H1 antagonist, a hematopoietic growth factor, and/or a blood transfusion.

In some embodiments, the additional therapeutic agent is an anti-cancer therapy. In some embodiments, the anti-cancer therapy comprises radiation therapy and/or a chemotherapeutic agent. In some embodiments, the chemotherapeutic agent is one or more of the following: a taxane, a platinum-based agent, an anthracycline, an anthraquinone, an alkylating agent, a HER2 targeting therapy, vinorelbine, a nucleoside analog, ixabepilone, eribulin, cytarabine, a hormonal therapy, capecitabine, lapatinib, 5-FU, vincristine, etoposide, or methotrexate.

A number of suitable anticancer agents are contemplated for use in the methods of the present disclosure. Indeed, the present disclosure contemplates, but is not limited to, administration of numerous anticancer agents such as: agents that induce apoptosis; polynucleotides (e.g., anti-sense, ribozymes, siRNA); polypeptides (e.g., enzymes and antibodies); biological mimetics; alkaloids; alkylating agents; antitumor antibiotics; antimetabolites; hormones; platinum compounds; monoclonal or polyclonal antibodies (e.g., antibodies conjugated with anticancer drugs, toxins, defensins), toxins; radionuclides; biological response modifiers (e.g., interferons (e.g., IFN-α) and interleukins (e.g., IL-2)); adoptive immunotherapy agents; hematopoietic growth factors; agents that induce tumor cell differentiation (e.g., all-trans-retinoic acid); gene therapy reagents (e.g., antisense therapy reagents and nucleotides); tumor vaccines; angiogenesis inhibitors; proteosome inhibitors: NF-KB modulators; anti-CDK compounds; HDAC inhibitors; and the like. Numerous other examples of chemotherapeutic compounds and anticancer therapies suitable for co-administration with the disclosed compounds are known to those skilled in the art.

In certain embodiments, anticancer agents comprise agents that induce or stimulate apoptosis. Agents that induce apoptosis include, but are not limited to, radiation (e.g., X-rays, gamma rays, UV); tumor necrosis factor (TNF)-related factors (e.g., TNF family receptor proteins, TNF family ligands, TRAIL, antibodies to TRAIL-R1 or TRAIL-R2); kinase inhibitors (e.g., epidermal growth factor receptor (EGFR) kinase inhibitor, vascular growth factor receptor (VGFR) kinase inhibitor, fibroblast growth factor receptor (FGFR) kinase inhibitor, platelet-derived growth factor receptor (PDGFR) kinase inhibitor, and Bcr-Abl kinase inhibitors (such as GLEEVEC)); antisense molecules; antibodies (e.g., HERCEPTIN, RITUXAN, ZEVALIN, and AVASTIN); anti-estrogens (e.g., raloxifene and tamoxifen); antiandrogens (e.g., flutamide, bicalutamide, finasteride, aminoglutethamide, ketoconazole, and corticosteroids); cyclooxygenase 2 (COX-2) inhibitors (e.g., celecoxib, meloxicam, NS-398, and non-steroidal anti-inflammatory drugs (NSAIDs)); anti-inflammatory drugs (e.g., butazolidin, DECADRON, DELTASONE, dexamethasone, dexamethasone intensol, DEXONE, HEXADROL, hydroxychloroquine, METICORTEN, ORADEXON, ORASONE, oxyphenbutazone, PEDIAPRED, phenylbutazone, PLAQUENIL, prednisolone, prednisone, PRELONE, and TANDEARIL); and cancer chemotherapeutic drugs (e.g., irinotecan (CAMPTOSAR), CPT-11, fludarabine (FLUDARA), dacarbazine (DTIC), dexamethasone, mitoxantrone, MYLOTARG, VP-16, cisplatin, carboplatin, oxaliplatin, 5-FU, doxorubicin, gemcitabine, bortezomib, gefitinib, bevacizumab, TAXOTERE or TAXOL); cellular signaling molecules; ceramides and cytokines; staurosporine, and the like.

In still other embodiments, the compositions and methods of the present disclosure provide an agent as described herein (e.g., an agent capable of inhibiting activity and/or expression of integrin α6) (e.g., an agent capable of inhibiting activity and/or expression of BCCs expressing integrin α6β1) (e.g., an agent capable of preventing expression of integrin α6 in BCCs) (e.g., an agent capable of preventing metastasis of BCCs expressing integrin α6 to LM cells) (e.g., an agent capable of preventing metastasis of BCCs expressing integrin α6β1 to LM cells) (e.g., an agent capable of inhibiting activity/expression of LM related GDNF signaling) (e.g., an agent capable of inhibiting activity/expression of LM related NCAM signaling) (e.g., an agent capable of inhibiting activity/expression of LM related GDNF) (e.g., an agent capable of inhibiting activity/expression of LM related NCAM) (e.g., an agent capable of inhibiting activity/expression of LM related NRTN) (e.g., an agent capable of inhibiting activity/expression of LM related ARTN) (e.g., an agent capable of inhibiting activity/expression of LM related PSPN) and at least one anti-hyperproliferative or antineoplastic agent selected from alkylating agents, antimetabolites, and natural products, e.g., herbs and other plant and/or animal derived compounds.

Alkylating agents suitable for use in the present compositions and methods include, but are not limited to: 1) nitrogen mustards (e.g., mechlorethamine, cyclophosphamide, ifosfamide, melphalan (L-sarcolysin); and chlorambucil); 2) ethylenimines and methylmelamines (e.g., hexamethylmelamine and thiotepa); 3) alkyl sulfonates (e.g., busulfan); 4) nitrosoureas (e.g., carmustine (BCNU); lomustine (CCNU); semustine (methyl-CCNU); and streptozocin (streptozotocin)); and 5) triazenes (e.g., dacarbazine (DTIC; dimethyltriazenoimid-azolecarboxamide).

In some embodiments, antimetabolites suitable for use in the present compositions and methods include, but are not limited to: 1) folic acid analogs (e.g., methotrexate (amethopterin)); 2) pyrimidine analogs (e.g., fluorouracil (5-fluorouracil; 5-FU), floxuridine (fluorode-oxvuridine; FudR), and cytarabine (cytosine arabinoside)); and 3) purine analogs (e.g., mercaptopurine (6-mercaptopurine; 6-MP), thioguanine (6-thioguanine; TG), and pentostatin (2′-deoxycoformycin)).

In still further embodiments, chemotherapeutic agents suitable for use in the compositions and methods of the present disclosure include, but are not limited to: 1) vinca alkaloids (e.g., vinblastine (VLB), vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin (cis-DDP) and carboplatin); 7) anthracenediones (e.g., mitoxantrone); 8) substituted ureas (e.g., hydroxyurea); 9) methylhydrazine derivatives (e.g., procarbazine (N-methylhydrazine; MIH)); 10) adrenocortical suppressants (e.g., mitotane (o,p′-DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide): and 17) gonadotropin-releasing hormone analogs (e.g., leuprolide).

Any oncolytic agent that is routinely used in a cancer therapy context finds use in the compositions and methods of the present disclosure. For example, the U.S. Food and Drug Administration maintains a formulary of oncolytic agents approved for use in the United States. International counterpart agencies to the U.S.F.D.A. maintain similar formularies. Table 1 provides a list of exemplary antineoplastic agents approved for use in the U.S. Those skilled in the art will appreciate that the “product labels” required on all U.S. approved chemotherapeutics describe approved indications, dosing information, toxicity data, and the like, for the exemplary agents.

TABLE 1

| | | |
|---|---|---|
| Aldesleukin | Proleukin | Chiron Corp., |
| (des-alanyl-1, serine-125 human interleukin-2) | | Emeryville, CA |
| Alemtuzumab | Campath | Millennium and ILEX |
| (IgG1κ anti CD52 antibody) | | Partners, LP, |
| | | Cambridge, MA |
| Alitretinoin | Panretin | Ligand Pharmaceuticals, |
| (9-cis-retinoic acid) | | Inc., San Diego CA |
| Allopurinol | Zyloprim | GlaxoSmithKline, |
| (1,5-dihydro-4 H-pyrazolo[3,4-d]pyrimidin-4- | | Research Triangle Park, |
| one monosodium salt) | | NC |
| Altretamine | Hexalen | US Bioscience, West |
| (N,N,N′,N′,N″,N″,-hexamethyl-1,3,5-triazine- | | Conshohocken, PA |
| 2,4,6-triamine) | | |
| Amifostine | Ethyol | US Bioscience |
| (ethanethiol, 2-[(3-aminopropyl)amino]-, | | |
| dihydrogen phosphate (ester)) | | |
| Anastrozole | Arimidex | AstraZeneca |
| (1,3-Benzenediacetonitrile, a,a,a′,a′- | | Pharmaceuticals, LP, |
| tetramethyl-5-(1H-1,2,4-triazol-1-ylmethyl)) | | Wilmington, DE |
| Arsenic trioxide | Trisenox | Cell Therapeutic, Inc., |
| | | Seattle, WA |
| Asparaginase | Elspar | Merck & Co., Inc., |
| (L-asparagine amidohydrolase, type EC-2) | | Whitehouse Station, NJ |
| BCG Live | TICE BCG | Organon Teknika, Corp., |
| (lyophilized preparation of an attenuated strain | | Durham, NC |
| of Mycobacterium bovis (Bacillus Calmette- | | |
| Gukin [BCG], substrain Montreal) | | |
| bexarotene capsules | Targretin | Ligand Pharmaceuticals |
| (4-[1-(5,6,7,8-tetrahydro-3,5,5,8,8- | | |
| pentamethyl-2-napthalenyl)ethenyl]benzoic acid) | | |
| bexarotene gel | Targretin | Ligand Pharmaceuticals |
| Bleomycin | Blenoxane | Bristol-Myers Squibb |
| (cytotoxic glycopeptide antibiotics produced by | | Co., NY, NY |
| Streptomyces verticillus; bleomycin A₂ and | | |
| bleomycin B₂) | | |
| Capecitabine | Xeloda | Roche |
| (5′-deoxy-5-fluoro-N-[(pentyloxy)carbonyl]- | | |
| cytidine) | | |
| Carboplatin | Paraplatin | Bristol-Myers Squibb |
| (platinum, diammine [1,1- | | |
| cyclobutanedicarboxylato(2-)-0,0′]-,(SP-4-2)) | | |
| Carmustine | BCNU, BiCNU | Bristol-Myers Squibb |
| (1,3-bis(2-chloroethyl)-1-nitrosourea) | | |
| Carmustine with Polifeprosan 20 Implant | Gliadel Wafer | Guilford |
| | | Pharmaceuticals, Inc., |
| | | Baltimore, MD |
| Celecoxib | Celebrex | Searle Pharmaceuticals, |
| (as 4-[5-(4-methylphenyl)-3-(trifluoromethyl)- | | England |
| 1H-pyrazol-1-yl] | | |
| benzenesulfonamide) | | |
| Chlorambucil | Leukeran | GlaxoSmithKline |
| (4-[bis(2chlorethyl)amino]benzenebutanoic acid) | | |
| Cisplatin | Platinol | Bristol-Myers Squibb |
| (PtCl₂H₆N₂) | | |
| Cladribine | Leustatin, 2- | R. W. Johnson |
| (2-chloro-2′-deoxy-b-D-adenosine) | CdA | Pharmaceutical |
| | | Research Institute, |
| | | Raritan, NJ |
| Cyclophosphamide | Cytoxan, | Bristol-Myers Squibb |
| (2-[bis(2-chloroethyl)amino]tetrahydro-2H- | Neosar | |
| 13,2-oxazaphosphorine 2-oxide monohydrate) | | |
| Cytarabine | Cytosar-U | Pharmacia & Upjohn |
| (1-b-D-Arabinofuranosylcytosine, C₉H₁₃N₃O₅) | | Company |
| cytarabine liposomal | DepoCyt | Skye Pharmaceuticals, |
| | | Inc., San Diego, CA |
| Dacarbazine | DTIC-Dome | Bayer AG, Leverkusen, |
| (5-(3,3-dimethyl-l-triazeno)-imidazole-4- | | Germany |
| carboxamide (DTIC)) | | |
| Dactinomycin, actinomycin D | Cosmegen | Merck |
| (actinomycin produced by Streptomyces | | |
| parvullus, C₆₂H₈₆N₁₂O₁₆) | | |
| Darbepoetin alfa | Aranesp | Amgen, Inc., Thousand |
| (recombinant peptide) | | Oaks, CA |
| daunorubicin liposomal | Danuo Xome | Nexstar |
| ((8S-cis)-8-acetyl-10-[(3-amino-2,3,6-trideoxy- | | Pharmaceuticals, Inc., |
| á-L-lyxo-hexopyranosyl)oxy]-7,8,9,10- | | Boulder, CO |
| tetrahydro-6,8,11-trihydroxy-1-methoxy-5,12- | | |
| naphthacenedione hydrochloride) | | |
| Daunorubicin HCl, daunomycin | Cerubidine | Wyeth Ayerst, Madison, |
| ((1S,3S)-3-Acetyl-1,2,3,4,6,11-hexahydro- | | NJ |
| 3,5,12-trihydroxy-10-methoxy-6,11-dioxo-1- | | |
| naphthacenyl 3-amino-2,3,6-trideoxy-(alpha)- | | |
| L-lyxo-hexopyranoside hydrochloride) | | |
| Denileukin diftitox | Ontak | Seragen, Inc., |
| (recombinant peptide) | | Hopkinton, MA |
| Dexrazoxane | Zinecard | Pharmacia & Upjohn |
| ((S)-4,4′-(1-methyl-1,2-ethanediyl)bis-2,6- | | Company |
| piperazinedione) | | |
| Docetaxel | Taxotere | Aventis |
| ((2R,3S)-N-carboxy-3-phenylisoserine, N-tert- | | Pharmaceuticals, Inc., |
| butyl ester, 13-ester with 5b-20-epoxy- | | Bridgewater, NJ |
| 12a,4,7b,10b,13a-hexahydroxytax-11-en-9-one | | |
| 4-acetate 2-benzoate, trihydrate) | | |
| Doxorubicin HCl | Adriamycin, | Pharmacia & Upjohn |
| (8S,10S)-10-[(3-amino-2,3,6-trideoxy-a-L- | Rubex | Company |
| lyxo-hexopyranosyl)oxy]-8-glycolyl-7,8,9,10- | | |
| tetrahydro-6,8,11-trihydroxy-1-methoxy-5,12- | | |
| naphthacenedione hydrochloride) | | |
| doxorubicin | Adriamycin PFS | Pharmacia & Upjohn |
| | Intravenous | Company |
| | injection | |
| doxorubicin liposomal | Doxil | Sequus Pharmaceuticals, |
| | | Inc., Menlo park, CA |
| dromostanolone propionate | Dromostanolone | Eli Lilly & Company, |
| (17b-Hydroxy-2a-methyl-5a-androstan-3-one | | Indianapolis, IN |
| propionate) | | |
| dromostanolone propionate | Masterone | Syntex, Corp., Palo |
| | injection | Alto, CA |
| Elliott′s B Solution | Elliott′s B | Orphan Medical, Inc |
| | Solution | |
| Epirubicin | Ellence | Pharmacia & Upjohn |
| ((8S-cis)-10-[(3-amino-2,3,6-trideoxy-a-L- | | Company |
| arabino-hexopyranosyl)oxy]-7,8,9,10- | | |
| tetrahydro-6,8,11-trihydroxy-8- | | |
| (hydroxyacetyl)-1-methoxy-5,12- | | |
| naphthacenedione hydrochloride) | | |
| Epoetin alfa (recombinant peptide) | Epogen | Amgen, Inc |
| Estramustine | Emcyt | Pharmacia & Upjohn |
| (estra-1,3,5(10)-triene-3,17-diol(17(beta))-,3- | | Company |
| [bis(2-chloroethyl)carbamate]17-(dihydrogen | | |
| phosphate), disodium salt, monohydrate, or | | |
| estradiol 3-[bis(2-chloroethyl)carbamate]17- | | |
| (dihydrogen phosphate), disodium salt, | | |
| monohydrate) | | |
| Etoposide phosphate | Etopophos | Bristol-Myers Squibb |
| (4′-Demethylepipodophyllotoxin 9-[4,6-O-(R)- | | |
| ethylidene-(beta)-D-glucopyranoside],4′- | | |
| (dihydrogen phosphate)) | | |
| etoposide, VP-16 | Vepesid | Bristol-Myers Squibb |
| (4′-demethylepipodophyllotoxin 9-[4,6-0-(R)- | | |
| ethylidene-(beta)-D-glucopyranoside]) | | |
| Exemestane | Aromasin | Pharmacia & Upjohn |
| (6-methylenandrosta-1,4-diene-3,17-dione) | | Company |
| Filgrastim (r-metHuG-CSF) | Neupogen | Amgen, Inc |
| floxuridine (intraarterial) | FUDR | Roche |
| (2′-deoxy-5-fluorouridine) | | |
| Fludarabine | Fludara | Berlex Laboratories, |
| (fluorinated nucleotide analog of the antiviral | | Inc., Cedar Knolls, NJ |
| agent vidarabine, 9-b-D- | | |
| arabinofuranosyladenine (ara-A)) | | |
| Fluorouracil, 5-FU | Adrucil | ICN Pharmaceuticals, |
| (5-fluoro-2,4(1H,3H)-pyrimidinedione) | | Inc., Humacao, Puerto |
| | | Rico |
| Fulvestrant | Faslodex | IPR Pharmaceuticals, |
| (7-alpha-[9-(4,4,5,5,5-penta | | Guayama, Puerto Rico |
| fluoropentylsulphinyl)nonyl]estra-1,3,5-(10)- | | |
| triene-3,17-beta-diol) | | |
| Gemcitabine | Gemzar | Eli Lilly |
| (2′-deoxy-2′,2′-difluorocytidine | | |
| monohydrochloride(b-isomer)) | | |
| Gemtuzumab Ozogamicin (anti-CD33 hP67.6) | Mylotarg | Wyeth Ayerst |
| Goserelin acetate | Zoladex | AstraZeneca |
| | Implant | Pharmaceuticals |
| Hydroxyurea | Hydrea | Bristol-Myers Squibb |
| Ibritumomab Tiuxetan | Zevalin | Biogen IDEC, Inc., |
| (immunoconjugate resulting from a thiourea | | Cambridge MA |
| covalent bond between the monoclonal | | |
| antibody Ibritumomab and the linker-chelator | | |
| tiuxetan [N-[2-bis(carboxymethyl)amino]-3-(p- | | |
| isothiocyanatophenyl)-propyl]-[N-[2- | | |
| bis(carboxymethyl)amino]-2-(methyl)- | | |
| ethyl]glycine) | | |
| Idarubicin | Idamycin | Pharmacia & Upjohn |
| (5,12-Naphthacenedione,9-acetyl-7-[(3- | | Company |
| amino-2,3,6-trideoxy-(alpha)-L-lyxo- | | |
| hexopyranosyl)oxy]-7,8,9,10-tetrahydro- | | |
| 6,9,11-trihydroxyhydrochloride, (7S-cis)) | | |
| Ifosfamide | IFEX | Bristol-Myers Squibb |
| (3-(2-chloroethyl)-2-[(2- | | |
| chloroethyl)amino]tetrahydro-2H-1,3,2- | | |
| oxazaphosphorine 2-oxide) | | |
| Imatinib Mesilate | Gleevec | Novartis AG, Basel, |
| (4-[(4-Methyl-1-piperazinyl)methyl]-N-[4- | | Switzerland |
| methyl-3-[[4-(3-pyridinyl)-2- | | |
| pyrimidinyl]amino]-phenyl]benzamide | | |
| methanesulfonate) | | |
| Interferon alfa-2a | Roferon-A | Hoffmann-La Roche, |
| (recombinant peptide) | | Inc., Nutley, NJ |
| Interferon alfa-2b | Intron A | Schering AG, Berlin, |
| (recombinant peptide) | (Lyophilized | Germany |
| | Betaseron) | |
| Irinotecan HCl | Camptosar | Pharmacia & Upjohn |
| | | Company |
| ((4S)-4,11-diethyl-4-hydroxy-9-[(4-piperi- | | |
| dinopiperidino)carbonyloxy]-1H-pyrano[3′,4′: | | |
| 6,7]indolizino[1,2-b]quinoline-3,14(4H,12H) | | |
| dione hydrochloride trihydrate) | | |
| Letrozole | Femara | Novartis |
| (4,4′-(1H-1,2,4-Triazol-1-ylmethylene) | | |
| dibenzonitrile) | | |
| Leucovorin | Wellcovorin, | Immunex, Corp., |
| (L-Glutamic acid, N[4[[(2amino-5-formyl- | Leucovorin | Seattle, WA |
| 1,4,5,6,7,8 hexahydro4oxo6- | | |
| pteridinyl)methyl]amino]benzoyl], calcium salt | | |
| (1:1)) | | |
| Levamisole HCl | Ergamisol | Janssen Research |
| ((−)-( S)-2,3,5,6-tetrahydro-6-phenylimidazo | | Foundation, Titusville, |
| [2,1-b] thiazole monohydrochloride | | NJ |
| C₁₁H₁₂N₂S•HCl) | | |
| Lomustine | CeeNU | Bristol-Myers Squibb |
| (1-(2-chloro-ethyl)-3-cyclohexyl-1-nitrosourea) | | |
| Meclorethamine, nitrogen mustard | Mustargen | Merck |
| (2-chloro-N-(2-chloroethyl)-N- | | |
| methylethanamine hydrochloride) | | |
| Megestrol acetate | Megace | Bristol-Myers Squibb |
| 17α(acetyloxy)-6-methylpregna-4,6-diene- | | |
| 3,20-dione | | |
| Melphalan, L-PAM | Alkeran | GlaxoSmithKline |
| (4-[bis(2-chloroethyl)amino]-L-phenylalanine) | | |
| Mercaptopurine, 6-MP | Purinethol | GlaxoSmithKline |
| (1,7-dihydro-6H-purine-6-thione monohydrate) | | |
| Mesna | Mesnex | Asta Medica |
| (sodium 2-mercaptoethane sulfonate) | | |
| Methotrexate | Methotrexate | Lederle Laboratories |
| (N-[4-[[(2,4-diamino-6- | | |
| pteridinyl)methyl]methylamino]benzoyl]-L- | | |
| glutamic acid) | | |
| Methoxsalen | Uvadex | Therakos, Inc., Way |
| (9-methoxy-7H-furo[3,2-g][1]-benzopyran-7-one) | | Exton, Pa |
| Mitomycin C | Mutamycin | Bristol-Myers Squibb |
| mitomycin C | Mitozytrex | SuperGen, Inc., Dublin, |
| | | CA |
| Mitotane | Lysodren | Bristol-Myers Squibb |
| (1,1-dichloro-2-(o-chlorophenyl)-2-(p- | | |
| chlorophenyl)ethane) | | |
| Mitoxantrone | Novantrone | Immunex Corporation |
| (1,4-dihydroxy-5,8-bis[[2-[(2- | | |
| hydroxyethyl)amino]ethyl]amino]-9,10- | | |
| anthracenedione dihydrochloride) | | |
| Nandrolone phenpropionate | Durabolin-50 | Organon, Inc., West |
| | | Orange, NJ |
| Nofetumomab | Verluma | Boehringer Ingelheim |
| | | Pharma KG, Germany |
| Oprelvekin | Neumega | Genetics Institute, Inc., |
| (IL-11) | | Alexandria, VA |
| Oxaliplatin | Eloxatin | Sanofi Synthelabo, Inc., |
| (cis-[(1R,2R)-1,2-cyclohexanediamine-N,N′] | | NY, NY |
| [oxalato(2-)-O,O′]platinum) | | |
| Paclitaxel | TAXOL | Bristol-Myers Squibb |
| (5β, 20-Epoxy-1,2a,4,7β,10β,13a- | | |
| hexahydroxytax-11-en-9-one 4,10-diacetate 2- | | |
| benzoate 13-ester with (2R,3S)-N-benzoyl-3- | | |
| phenylisoserine) | | |
| Pamidronate | Aredia | Novartis |
| (phosphonic acid (3-amino-1- | | |
| hydroxypropylidene) bis-, disodium salt, | | |
| pentahydrate, (APD)) | | |
| Pegademase | Adagen | Enzon Pharmaceuticals, |
| ((monomethoxypolyethylene glycol | (Pegademase | Inc., Bridgewater, NJ |
| succinimidyl)11-17-adenosine deaminase) | Bovine) | |
| Pegaspargase | Oncaspar | Enzon |
| (monomethoxypolyethylene glycol | | |
| succinimidyl L-asparaginase) | | |
| Pegfilgrastim | Neulasta | Amgen, Inc |
| (covalent conjugate of recombinant methionyl | | |
| human G-CSF (Filgrastim) and | | |
| monomethoxypolyethylene glycol) | | |
| Pentostatin | Nipent | Parke-Davis |
| | | Pharmaceutical Co., |
| | | Rockville, MD |
| Pipobroman | Vercyte | Abbott Laboratories, |
| | | Abbott Park, IL |
| Plicamycin, Mithramycin | Mithracin | Pfizer, Inc., NY, NY |
| (antibiotic produced by Streptomyces plicatus) | | |
| Porfimer sodium | Photofrin | QLT Phototherapeutics, |
| | | Inc., Vancouver, |
| | | Canada |
| Procarbazine | Matulane | Sigma Tau |
| (N-isopropyl-μ-(2-methylhydrazino)-p- | | Pharmaceuticals, Inc., |
| toluamide monohydrochloride) | | Gaithersburg, MD |
| Quinacrine | Atabrine | Abbott Labs |
| (6-chloro-9-(1-methyl-4-diethyl-amine) | | |
| butylamino-2-methoxyacridine) | | |
| Rasburicase (recombinant peptide) | Elitek | Sanofi-Synthelabo, Inc., |
| Rituximab | Rituxan | Genentech, Inc., South |
| (recombinant anti-CD20 antibody) | | San Francisco, CA |
| Sargramostim | Prokine | Immunex Corp |
| (recombinant peptide) | | |
| Streptozocin | Zanosar | Pharmacia & Upjohn |
| (streptozocin 2-deoxy-2- | | Company |
| [[(methylnitrosoamino)carbonyl]amino]-a(and | | |
| b)-D-glucopyranose and 220 mg citric acid | | |
| anhydrous) | | |
| Talc (Mg₃Si₄O₁₀(OH)₂) | Sclerosol | Bryan, Corp., Woburn, MA |
| Tamoxifen | Nolvadex | AstraZeneca |
| ((Z)2-[4-(1,2-diphenyl-1-butenyl) phenoxy]-N, | | Pharmaceuticals |
| N-dimethylethanamine 2-hydroxy-1,2,3- | | |
| propanetricarboxylate (1:1)) | | |
| Temozolomide | Temodar | Schering |
| (3,4-dihydro-3-methyl-4-oxoimidazo[5,1-d]-as- | | |
| tetrazine-8-carboxamide) | | |
| teniposide, VM-26 | Vumon | Bristol-Myers Squibb |
| (4′-demethylepipodophyllotoxin 9-[4,6-0-(R)- | | |
| 2-thenylidene-(beta)-D-glucopyranoside]) | | |
| Testolactone | Teslac | Bristol-Myers Squibb |
| (13-hydroxy-3-oxo-13,17-secoandrosta-1,4- | | |
| dien-17-oic acid [dgr]-lactone) | | |
| Thioguanine, 6-TG | Thioguanine | GlaxoSmithKline |
| (2-amino-1,7-dihydro-6H-purine-6-thione) | | |
| Thiotepa | Thioplex | Immunex Corporation |
| (Aziridine, 1,1′,1″-phosphinothioylidynetris-, or | | |
| Tris (1-aziridinyl) phosphine sulfide) | | |
| Topotecan HCl | Hycamtin | GlaxoSmithKline |
| ((S)-10-[(dimethylamino) methyl]-4-ethyl-4,9- | | |
| dihydroxy-1H-pyrano[3′,4′: 6,7] indolizino | | |
| [1,2-b] quinoline-3,14-(4H,12H)-dione | | |
| monohydrochloride) | | |
| Toremifene | Fareston | Roberts Pharmaceutical |
| (2-(p-[(Z)-4-chloro-1,2-diphenyl-1-butenyl]- | | Corp., Eatontown, NJ |
| phenoxy)-N,N-dimethylethylamine citrate (1:1)) | | |
| Tositumomab, I 131 Tositumomab | Bexxar | Corixa Corp., Seattle, |
| (recombinant murine immunotherapeutic | | WA |
| monoclonal IgG₂ₐ lambda anti-CD20 antibody | | |
| (I 131 is a radioimmunotherapeutic antibody)) | | |
| Trastuzumab | Herceptin | Genentech, Inc |
| (recombinant monoclonal IgG₁ kappa anti- | | |
| HER2 antibody) | | |
| Tretinoin, ATRA (all-trans retinoic acid) | Vesanoid | Roche |
| Uracil Mustard | Uracil Mustard | Roberts Labs |
| | Capsules | |
| Valrubicin, N-trifluoroacetyladriamycin-14- | Valstar | Anthra -- > Medeva |
| valerate | | |
| ((2S-cis)-2-[1,2,3,4,6,11-hexahydro-2,5,12- | | |
| trihydroxy-7 methoxy-6,11-dioxo-[[4 2,3,6- | | |
| trideoxy-3-[(trifluoroacetyl)-amino-α-L-lyxo- | | |
| hexopyranosyl]oxyl]-2-naphthacenyl]-2- | | |
| oxoethyl pentanoate) | | |
| Vinblastine, Leurocristine | Velban | Eli Lilly |
| (C₄₆H₅₆N₄O₁₀•H₂SO₄) | | |
| Vincristine | Oncovin | Eli Lilly |
| (C₄₆H₅₆N₄O₁₀•H₂SO₄) | | |
| Vinorelbine | Navelbine | GlaxoSmithKline |
| (3′,4′-didehydro-4′-deoxy-C′- | | |
| norvincaleukoblastine [R-(R*,R*)-2,3- | | |
| dihydroxybutanedioate (1:2)(salt)]) | | |
| Zoledronate, Zoledronic acid | Zometa | Novartis |
| ((1-Hydroxy-2-imidazol-1-yl-phosphonoethyl) | | |
| phosphonic acid monohydrate) | | |

Anticancer agents further include compounds which have been identified to have anticancer activity. Examples include, but are not limited to, 3-AP, 12-O-tetradecanoylphorbol-13-acetate, 17AAG, 852A, ABI-007, ABR-217620, ABT-751, ADI-PEG 20, AE-941, AG-013736, AGRO100, alanosine, AMG 706, antibody G250, antineoplastons, AP23573, apaziquone, APC8015, atiprimod, ATN-161, atrasenten, azacitidine, BB-10901, BCX-1777, bevacizumab, BG00001, bicalutamide, BMS 247550, bortezomib, bryostatin-1, buserelin, calcitriol, CCI-779, CDB-2914, cefixime, cetuximab, CG0070, cilengitide, clofarabine, combretastatin A4 phosphate, CP-675,206, CP-724,714, CpG 7909, curcumin, decitabine, DENSPM, doxercalciferol, E7070, E7389, ecteinascidin 743, efaproxiral, eflornithine, EKB-569, enzastaurin, erlotinib, exisulind, fenretinide, flavopiridol, fludarabine, flutamide, fotemustine, FR901228, G17DT, galiximab, gefitinib, genistein, glufosfamide, GTI-2040, histrelin, HKI-272, homoharringtonine, HSPPC-96, hul4.18-interleukin-2 fusion protein, HuMax-CD4, iloprost, imiquimod, infliximab, interleukin-12, IPI-504, irofulven, ixabepilone, lapatinib, lenalidomide, lestaurtinib, leuprolide, LMB-9 immunotoxin, lonafarnib, luniliximab, mafosfamide, MB07133, MDX-010, MLN2704, monoclonal antibody 3F8, monoclonal antibody J591, motexafin, MS-275, MVA-MUCT-IL2, nilutamide, nitrocamptothecin, nolatrexed dihydrochloride, nolvadex, NS-9, O6-benzylguanine, oblimersen sodium, ONYX-015, oregovomab, OSI-774, panitumumab, paraplatin, PD-0325901, pemetrexed, PHY906, pioglitazone, pirfenidone, pixantrone, PS-341, PSC 833, PXD101, pyrazoloacridine, R115777, RAD001, ranpirnase, rebeccamycin analogue, rhuAngiostatin protein, rhuMab 2C4, rosiglitazone, rubitecan, S-1, S-8184, satraplatin, SB-, 15992, SGN-0010, SGN-40, sorafenib, SR31747A, ST1571, SU011248, suberoylanilide hydroxamic acid, suramin, talabostat, talampanel, tariquidar, temsirolimus, TGFa-PE38 immunotoxin, thalidomide, thymalfasin, tipifamib, tirapazamine, TLK286, trabectedin, trimetrexate glucuronate, TroVax, UCN-1, valproic acid, vinflunine, VNP40101M, volociximab, vorinostat, VX-680, ZD1839, ZD6474, zileuton, and zosuquidar trihydrochloride.

For a more detailed description of anticancer agents and other therapeutic agents, those skilled in the art are referred to any number of instructive manuals including, but not limited to, the Physician's Desk Reference and to Goodman and Gilman's “Pharmaceutical Basis of Therapeutics” tenth edition, Eds. Hardman et al., 2002.

The present disclosure provides methods for administering an agent as described herein (e.g., an agent capable of inhibiting activity and/or expression of integrin α6) (e.g., an agent capable of inhibiting activity and/or expression of BCCs expressing integrin α6β1) (e.g., an agent capable of preventing expression of integrin α6 in BCCs) (e.g., an agent capable of preventing metastasis of BCCs expressing integrin α6 to LM cells) (e.g., an agent capable of preventing metastasis of BCCs expressing integrin α6β1 to LM cells) (e.g., an agent capable of inhibiting activity/expression of LM related GDNF signaling) (e.g., an agent capable of inhibiting activity/expression of LM related NCAM signaling) (e.g., an agent capable of inhibiting activity/expression of LM related GDNF) (e.g., an agent capable of inhibiting activity/expression of LM related NCAM) (e.g., an agent capable of inhibiting activity/expression of LM related NRTN) (e.g., an agent capable of inhibiting activity/expression of LM related ARTN) (e.g., an agent capable of inhibiting activity/expression of LM related PSPN) with radiation therapy. The disclosure is not limited by the types, amounts, or delivery and administration systems used to deliver the therapeutic dose of radiation to the subject. For example, the subject may receive photon radiotherapy, particle beam radiation therapy, other types of radiotherapies, and combinations thereof. In some embodiments, the radiation is delivered to the subject using a linear accelerator. In still other embodiments, the radiation is delivered using a gamma knife.

The source of radiation can be external or internal to the subject. External radiation therapy is most common and involves directing a beam of high-energy radiation to a tumor site through the skin using, for instance, a linear accelerator. While the beam of radiation is localized to the tumor site, it is nearly impossible to avoid exposure of normal, healthy tissue. However, external radiation is usually well tolerated by subjects. Internal radiation therapy involves implanting a radiation-emitting source, such as beads, wires, pellets, capsules, particles, and the like, inside the body at or near the tumor site including the use of delivery systems that specifically target cancer cells, e.g., using particles attached to cancer cell binding ligands. Such implants can be removed following treatment, or left in the body inactive. Types of internal radiation therapy include, but are not limited to, brachytherapy, interstitial irradiation, intracavity irradiation, radioimmunotherapy, and the like.

The subject may optionally receive radiosensitizers (e.g., metronidazole, misonidazole, intra-arterial Budr, intravenous iododeoxyuridine (IudR), nitroimidazole, 5-substituted-4-nitroimidazoles, 2H-isoindolediones, [[(2-bromoethyl)-amino]methyl]-nitro-1H-imidazole-1-ethanol, nitroaniline derivatives, DNA-affinic hypoxia selective cytotoxins, halogenated DNA ligand, 1,2,4 benzotriazine oxides, 2-nitroimidazole derivatives, fluorine-containing nitroazole derivatives, benzamide, nicotinamide, acridine-intercalator, 5-thiotretrazole derivative, 3-nitro-1,2,4-triazole, 4,5-dinitroimidazole derivative, hydroxylated texaphrins, cisplatin, mitomycin, tiripazamine, nitrosourea, mercaptopurine, methotrexate, fluorouracil, bleomycin, vincristine, carboplatin, epirubicin, doxorubicin, cyclophosphamide, vindesine, etoposide, paclitaxel, heat (hyperthermia), and the like), radioprotectors (e.g., cysteamine, aminoalkyl dihydrogen phosphorothioates, amifostine (WR 2721), IL-1, IL-6, and the like). Radiosensitizers enhance the killing of tumor cells. Radioprotectors protect healthy tissue from the harmful effects of radiation.

Any type of radiation can be administered to an subject, so long as the dose of radiation is tolerated by the subject without unacceptable negative side-effects. Suitable types of radiotherapy include, for example, ionizing (electromagnetic) radiotherapy, e.g., X-rays or gamma rays, or particle beam radiation therapy, e.g., high linear energy radiation. Ionizing radiation is defined as radiation comprising particles or photons that have sufficient energy to produce ionization, i.e., gain or loss of electrons (as described in, for example, U.S. Pat. No. 5,770,581 incorporated herein by reference in its entirety). The effects of radiation can be at least partially controlled by the clinician. In one embodiment, the dose of radiation is fractionated for maximal target cell exposure and reduced toxicity.

In one embodiment, the total dose of radiation administered to s subject is about 0.01 Gray (Gy) to about 100 Gy. In another embodiment, about 10 Gy to about 65 Gy (e.g., about 15 Gy, 20 Gy, 25 Gy, 30 Gy, 35 Gy, 40 Gy, 45 Gy, 50 Gy, 55 Gy, or 60 Gy) are administered over the course of treatment. While in some embodiments a complete dose of radiation can be administered over the course of one day, the total dose is ideally fractionated and administered over several days. Desirably, radiotherapy is administered over the course of at least about 3 days, e.g., at least 5, 7, 10, 14, 17, 21, 25, 28, 32, 35, 38, 42, 46, 52, or 56 days (about 1-8 weeks). Accordingly, a daily dose of radiation will comprise approximately 1-5 Gy (e.g., about 1 Gy, 1.5 Gy, 1.8 Gy, 2 Gy, 2.5 Gy, 2.8 Gy, 3 Gy, 3.2 Gy, 3.5 Gy, 3.8 Gy, 4 Gy, 4.2 Gy, or 4.5 Gy), or 1-2 Gy (e.g., 1.5-2 Gy). The daily dose of radiation should be sufficient to induce destruction of the targeted cells. If stretched over a period, in one embodiment, radiation is not administered every day, thereby allowing the animal to rest and the effects of the therapy to be realized. For example, radiation desirably is administered on 5 consecutive days, and not administered on 2 days, for each week of treatment, thereby allowing 2 days of rest per week. However, radiation can be administered 1 day/week, 2 days/week, 3 days/week, 4 days/week, 5 days/week. 6 days/week, or all 7 days/week, depending on the animal's responsiveness and any potential side effects. Radiation therapy can be initiated at any time in the therapeutic period. In one embodiment, radiation is initiated in week 1 or week 2, and is administered for the remaining duration of the therapeutic period. For example, radiation is administered in weeks 1-6 or in weeks 2-6 of a therapeutic period comprising 6 weeks for treating, for instance, a solid tumor. Alternatively, radiation is administered in weeks 1-5 or weeks 2-5 of a therapeutic period comprising 5 weeks. These exemplary radiotherapy administration schedules are not intended, however, to limit the present disclosure.

In some embodiments of the present disclosure, an agent as described herein (e.g., an agent capable of inhibiting activity and/or expression of integrin α6) (e.g., an agent capable of inhibiting activity and/or expression of BCCs expressing integrin α6β1) (e.g., an agent capable of preventing expression of integrin α6 in BCCs) (e.g., an agent capable of preventing metastasis of BCCs expressing integrin α6 to LM cells) (e.g., an agent capable of preventing metastasis of BCCs expressing integrin α6β1 to LM cells) (e.g., an agent capable of inhibiting activity/expression of LM related GDNF signaling) (e.g., an agent capable of inhibiting activity/expression of LM related NCAM signaling) (e.g., an agent capable of inhibiting activity/expression of LM related GDNF) (e.g., an agent capable of inhibiting activity/expression of LM related NCAM) (e.g., an agent capable of inhibiting activity/expression of LM related NRTN) (e.g., an agent capable of inhibiting activity/expression of LM related ARTN) (e.g., an agent capable of inhibiting activity/expression of LM related PSPN) and one or more anticancer agents are administered to a subject under one or more of the following conditions: at different periodicities, at different durations, at different concentrations, by different administration routes, etc. In some embodiments, the agent described herein is administered prior to the anticancer agent, e.g., 0.5, 1, 2, 3, 4, 5, 10, 12, or 18 hours, 1, 2, 3, 4, 5, or 6 days, or 1, 2, 3, or 4 weeks prior to the administration of anticancer agent. In some embodiments, the agent described herein is administered after the anticancer agent, e.g., 0.5, 1, 2, 3, 4, 5, 10, 12, or 18 hours, 1, 2, 3, 4, 5, or 6 days, or 1, 2, 3, or 4 weeks after the administration of the anticancer agent. In some embodiments, the agent described herein and the anticancer agent are administered concurrently but on different schedules, e.g., the compound is administered daily while the therapeutic or anticancer agent is administered once a week, once every two weeks, once every three weeks, or once every four weeks. In other embodiments, the agent described herein administered once a week while the anticancer agent is administered daily, once a week, once every two weeks, once every three weeks, or once every four weeks.

Compositions within the scope of this disclosure include all compositions wherein the agent as described herein (e.g., an agent capable of inhibiting activity and/or expression of integrin α6) (e.g., an agent capable of inhibiting activity and/or expression of BCCs expressing integrin α6β1) (e.g., an agent capable of preventing expression of integrin α6 in BCCs) (e.g., an agent capable of preventing metastasis of BCCs expressing integrin α6 to LM cells) (e.g., an agent capable of preventing metastasis of BCCs expressing integrin α6β1 to LM cells) (e.g., an agent capable of inhibiting activity/expression of LM related GDNF signaling) (e.g., an agent capable of inhibiting activity/expression of LM related NCAM signaling) (e.g., an agent capable of inhibiting activity/expression of LM related GDNF) (e.g., an agent capable of inhibiting activity/expression of LM related NCAM) (e.g., an agent capable of inhibiting activity/expression of LM related NRTN) (e.g., an agent capable of inhibiting activity/expression of LM related ARTN) (e.g., an agent capable of inhibiting activity/expression of LM related PSPN) is contained in an amount which is effective to achieve its intended purpose. While individual needs vary, determination of optimal ranges of effective amounts of each component is within the skill of the art. Typically, the agent may be administered to subjects, e.g., human cancer patients, orally at a dose of 0.0025 to 100 mg/kg, or an equivalent amount of the pharmaceutically acceptable salt thereof, per day of the body weight of the mammal being treated for disorders responsive to induction of apoptosis. In one embodiment, about 0.01 to about 25 mg/kg of the agent is orally administered to treat, ameliorate, or prevent LMD. For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 1000 mg, for example, about 0.1 to about 100 mg of the agent. The unit dose may be administered one or more times daily as one or more tablets or capsules each containing from about 0.1 to about 10 mg, conveniently about 0.25 to 50 mg of ESK981.

In a topical formulation, the agent as described herein (e.g., an agent capable of inhibiting activity and/or expression of integrin α6) (e.g., an agent capable of inhibiting activity and/or expression of BCCs expressing integrin α6β1) (e.g., an agent capable of preventing expression of integrin α6 in BCCs) (e.g., an agent capable of preventing metastasis of BCCs expressing integrin 06 to LM cells) (e.g., an agent capable of preventing metastasis of BCCs expressing integrin α6β1 to LM cells) (e.g., an agent capable of inhibiting activity/expression of LM related GDNF signaling) (e.g., an agent capable of inhibiting activity/expression of LM related NCAM signaling) (e.g., an agent capable of inhibiting activity/expression of LM related GDNF) (e.g., an agent capable of inhibiting activity/expression of LM related NCAM) (e.g., an agent capable of inhibiting activity/expression of LM related NRTN) (e.g., an agent capable of inhibiting activity/expression of LM related ARTN) (e.g., an agent capable of inhibiting activity/expression of LM related PSPN) may be present at a concentration of about 0.01 to 100 mg per gram of carrier. In a one embodiment, the agent is present at a concentration of about 0.07-1.0 mg/ml, for example, about 0.1-0.5 mg/ml, and in one embodiment, about 0.4 mg/ml.

In some embodiments, the agent as described herein (e.g., an agent capable of inhibiting activity and/or expression of integrin α6) (e.g., an agent capable of inhibiting activity and/or expression of BCCs expressing integrin α6β1) (e.g., an agent capable of preventing expression of integrin α6 in BCCs) (e.g., an agent capable of preventing metastasis of BCCs expressing integrin α6 to LM cells) (e.g., an agent capable of preventing metastasis of BCCs expressing integrin α6β1 to LM cells) (e.g., an agent capable of inhibiting activity/expression of LM related GDNF signaling) (e.g., an agent capable of inhibiting activity/expression of LM related NCAM signaling) (e.g., an agent capable of inhibiting activity/expression of LM related GDNF) (e.g., an agent capable of inhibiting activity/expression of LM related NCAM) (e.g., an agent capable of inhibiting activity/expression of LM related NRTN) (e.g., an agent capable of inhibiting activity/expression of LM related ARTN) (e.g., an agent capable of inhibiting activity/expression of LM related PSPN) may be administered as part of a pharmaceutical formulation containing suitable pharmaceutically acceptable carriers comprising excipients and auxiliaries which facilitate processing of the agent into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of the agent, together with the excipient.

The pharmaceutical compositions comprising an agent as described herein (e.g., an agent capable of inhibiting activity and/or expression of integrin α6) (e.g., an agent capable of inhibiting activity and/or expression of BCCs expressing integrin α6β1) (e.g., an agent capable of preventing expression of integrin α6 in BCCs) (e.g., an agent capable of preventing metastasis of BCCs expressing integrin α6 to LM cells) (e.g., an agent capable of preventing metastasis of BCCs expressing integrin α6β1 to LM cells) (e.g., an agent capable of inhibiting activity/expression of LM related GDNF signaling) (e.g., an agent capable of inhibiting activity/expression of LM related NCAM signaling) (e.g., an agent capable of inhibiting activity/expression of LM related GDNF) (e.g., an agent capable of inhibiting activity/expression of LM related NCAM) (e.g., an agent capable of inhibiting activity/expression of LM related NRTN) (e.g., an agent capable of inhibiting activity/expression of LM related ARTN) (e.g., an agent capable of inhibiting activity/expression of LM related PSPN) may be administered to any subject which may experience the beneficial effects of such an agent. Foremost among such subjects are mammals, e.g., humans, although the disclosure is not intended to be so limited. Other subjects include veterinary animals (cows, sheep, pigs, horses, dogs, cats and the like).

An agent as described herein (e.g., an agent capable of inhibiting activity and/or expression of integrin α6) (e.g., an agent capable of inhibiting activity and/or expression of BCCs expressing integrin α6β1) (e.g., an agent capable of preventing expression of integrin α6 in BCCs) (e.g., an agent capable of preventing metastasis of BCCs expressing integrin α6 to LM cells) (e.g., an agent capable of preventing metastasis of BCCs expressing integrin α6β1 to LM cells) (e.g., an agent capable of inhibiting activity/expression of LM related GDNF signaling) (e.g., an agent capable of inhibiting activity/expression of LM related NCAM signaling) (e.g., an agent capable of inhibiting activity/expression of LM related GDNF) (e.g., an agent capable of inhibiting activity/expression of LM related NCAM) (e.g., an agent capable of inhibiting activity/expression of LM related NRTN) (e.g., an agent capable of inhibiting activity/expression of LM related ARTN) (e.g., an agent capable of inhibiting activity/expression of LM related PSPN) and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present disclosure are manufactured in a manner which is itself known, for example, by means of conventional mixing, granulating, dragee-making, dissolving, or lyophilizing processes. Thus, pharmaceutical preparations for oral use can be obtained by combining agent as described herein (e.g., an agent capable of inhibiting activity and/or expression of integrin α6) (e.g., an agent capable of inhibiting activity and/or expression of BCCs expressing integrin α6$1) (e.g., an agent capable of preventing expression of integrin α6 in BCCs) (e.g., an agent capable of preventing metastasis of BCCs expressing integrin α6 to LM cells) (e.g., an agent capable of preventing metastasis of BCCs expressing integrin α6β1 to LM cells) (e.g., an agent capable of inhibiting activity/expression of LM related GDNF signaling) (e.g., an agent capable of inhibiting activity/expression of LM related NCAM signaling) (e.g., an agent capable of inhibiting activity/expression of LM related GDNF) (e.g., an agent capable of inhibiting activity/expression of LM related NCAM) (e.g., an agent capable of inhibiting activity/expression of LM related NRTN) (e.g., an agent capable of inhibiting activity/expression of LM related ARTN) (e.g., an agent capable of inhibiting activity/expression of LM related PSPN) with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this disclosure are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C12). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762; each herein incorporated by reference in its entirety.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight. Lotions may be conveniently prepared by dissolving the active ingredient, in a suitable high molecular weight alcohol such as propylene glycol or polyethylene glycol.

In another embodiment, the present disclosure provides methods of treating a subject having LMD comprising (a) determining whether a biomarker is present or absent in a biological sample taken from the subject; and (b) administering a therapeutically effective amount of an agent as described herein (e.g., an agent capable of inhibiting activity and/or expression of integrin α6) (e.g., an agent capable of inhibiting activity and/or expression of BCCs expressing integrin α6β1) (e.g., an agent capable of preventing expression of integrin α6 in BCCs) (e.g., an agent capable of preventing metastasis of BCCs expressing integrin α6 to LM cells) (e.g., an agent capable of preventing metastasis of BCCs expressing integrin α6β1 to LM cells) (e.g., an agent capable of inhibiting activity/expression of LM related GDNF signaling) (e.g., an agent capable of inhibiting activity/expression of LM related NCAM signaling) (e.g., an agent capable of inhibiting activity/expression of LM related GDNF) (e.g., an agent capable of inhibiting activity/expression of LM related NCAM) (e.g., an agent capable of inhibiting activity/expression of LM related NRTN) (e.g., an agent capable of inhibiting activity/expression of LM related ARTN) (e.g., an agent capable of inhibiting activity/expression of LM related PSPN) and, optionally, one or more additional anticancer agents to the subject if the biomarker is present in the biological sample.

In some embodiments, the biomarker is one or more of the following: the presence of BCC expression of integrin α6, the presence of activity of BCCs expressing integrin α6 in LM cells, the presence of abluminal vascular LM invasion by BCCs expressing integrin α6, the presence of engagement of BCCs having integrin α6 expression with vascular basement membrane laminin in LM cells, and the presence of encasement of BCC having integrin α6 expression in perivascular CSF1R+ meningeal macrophages.

Biomarker standards can be predetermined, determined concurrently, or determined after a biological sample is obtained from the subject. Biomarker standards for use with the methods described herein can, for example, include data from samples from subjects without LMD; data from samples from subjects with LMD. Comparisons can be made to establish predetermined threshold biomarker standards for different classes of subjects, e.g., diseased vs. undiseased subjects. The standards can be run in the same assay or can be known standards from a previous assay.

A biomarker is differentially present between different phenotypic status groups if the mean or median expression or mutation levels of the biomarker is calculated to be different, i.e., higher or lower, between the groups. Thus, biomarkers provide an indication that a subject, e.g., an LMD patient, belongs to one phenotypic status or another.

The determination of the expression level or mutation status of a biomarker in a subject can be performed using any of the many methods known in the art. Examples include, but are not limited to, PCR (polymerase chain reaction), or RT-PCR, flow cytometry, Northern blot, Western blot, ELISA (enzyme linked immunosorbent assay), RIA (radioimmunoassay), gene chip analysis of RNA expression, immunohistochemistry or immunofluorescence. See, e.g., Slagle et al. Cancer 83:1401 (1998); Hudlebusch et al., Clin Cancer Res 17:2919-2933 (2011). Certain embodiments of the disclosure include methods wherein biomarker RNA expression (transcription) is determined. Other embodiments of the disclosure include methods wherein protein expression in the biological sample is determined. See, e.g., Harlow et al., Antibodies: A Laboratory Manual, Cold Spring Harbor Laboratory, Cold Spring Harbor, NY, (1988); Ausubel et al., Current Protocols in Molecular Biology, John Wiley & Sons, New York 3rd Edition, (1995); Kamel and Al-Amodi, Genomics Proteomics Bioinformatics 15:220-235 (2017). For northern blot or RT-PCR analysis, RNA is isolated from the tumor tissue sample using RNAse free techniques. Such techniques are commonly known in the art.

In one embodiment of the disclosure, a biological sample of LM cells is obtained from the subject and the biological sample is assayed for determination of a biomarker presence or absence.

In certain embodiments, the present invention provides a method for determining if a subject (e.g., a human subject suffering from breast cancer) is suffering from or at risk of suffering from metastasis comprising obtaining a biological sample from the subject, detecting the presence or absence of BCCs expressing integrin α6, and determining that the subject is suffering from or at risk of suffering from metastasis if BCCs expressing integrin α6 are detected in the biological sample. In some embodiments, the metastatic cancer comprises systemic and brain parenchymal metastases. In some embodiments, the metastatic cancer comprises LMD.

In certain embodiments, the present invention provides a method for preventing and/or treating metastatic cancer in a subject, the method comprising, consisting of, or consisting essentially of administering to the subject a therapeutically effective amount of an inhibitory molecule, or a pharmaceutical composition thereof, capable of inhibiting and/or downregulating the binding, signaling, and/or function of a molecule selected from the group consisting of integrin α6, GDNF, NCAM, GDNF/NCAM complex, macrophages and/or microglia, and combinations thereof. In some embodiments, the metastatic cancer comprises systemic and brain parenchymal metastases. In some embodiments, the metastatic cancer comprises LMD.

In certain embodiments, the present invention provides a method for preventing and/or treating systemic and brain parenchymal metastases in a subject, the method comprising, consisting of, or consisting essentially of administering to the subject a therapeutically effective amount of an inhibitory molecule, or a pharmaceutical composition thereof, capable of inhibiting and/or downregulating the binding, signaling, and/or function of a molecule selected from the group consisting of: integrin α6, GDNF, NCAM, GDNF/NCAM complex, macrophages and/or microglia, and combinations thereof such that the systemic and brain parenchymal metastases is prevented and/or treated. In some embodiments, the metastatic cancer comprises systemic and brain parenchymal metastases. In some embodiments, the metastatic cancer comprises LMD.

Another aspect of the present disclosure provides a method of preventing and/or treating leptomeningeal disease in a subject, the method comprising, consisting of, or consisting essentially of administering to the subject a therapeutically effective amount of an inhibitory molecule, or a pharmaceutical composition thereof, capable of inhibiting and/or downregulating the binding, signaling, and/or function of a molecule selected from the group consisting of integrin α6, GDNF, NCAM, GDNF/NCAM complex, macrophages and/or microglia, and combinations thereof such that the leptomeningeal disease is prevented and/or treated.

In some embodiments, the inhibitory molecule is selected from the group consisting of an antibody, oligonucleotide, aptamer, small inhibitory molecule, and combinations thereof.

In some embodiments, the inhibitory molecule comprises an antibody. In one embodiment, the antibody is specific to α6β1. In another embodiment, the antibody is specific to GDNF. In another embodiment, the antibody is specific to NCAM. In yet another embodiment, the antibody is capable of inhibiting and/or disrupting the GDNF/NCAM complex.

In other embodiments, the inhibitory molecule comprises a small inhibitory molecule. In one embodiment, the small inhibitory molecule is capable of inhibiting and/or disrupting the GDNF/NCAM complex and/or signaling pathway.

In another embodiment, the inhibitory molecule is provided in a vector that is administered to the subject.

Another aspect of the present disclosure provides an inhibitory molecule as provided herein and a pharmaceutically acceptable carrier, diluent, and/or excipient.

Another aspect of the present disclosure provides a method of predicting a subject at risk of developing and/or identifying a subject suffering from, metastatic cancer, the method comprising, consisting of, or consisting essentially of: (i) obtaining a biological sample from the subject; (ii) identifying the presence of one or more biomarkers associated with metastatic cancer and comparing that to a control sample; (iii) if the biomarker is determined to be present, administering an anticancer therapy to the subject.

In one embodiment, the biomarker is selected from the group consisting of integrin α6, NCAM, and combinations thereof.

In some embodiments, the metastatic cancer comprises systemic and brain parenchymal metastases. In other embodiments, the metastatic cancer comprises leptomeningeal disease.

Another aspect of the present disclosure provides a method of preventing and/or treating metastatic cancer in a subject, the method comprising, consisting of, or consisting essentially of administering to the subject a therapeutically effective amount of an inhibitory molecule, or a pharmaceutical composition thereof, capable of inhibiting and/or downregulating the binding, signaling, and/or function of a molecule selected from the group consisting of: integrin α6, GDNF, NCAM, GDNF/NCAM complex, macrophages and/or microglia, and combinations thereof.

Another aspect of the present disclosure provides a method of preventing and/or treating systemic and brain parenchymal metastases in a subject, the method comprising, consisting of, or consisting essentially of administering to the subject a therapeutically effective amount of an inhibitory molecule, or a pharmaceutical composition thereof, capable of inhibiting and/or downregulating the binding, signaling, and/or function of a molecule selected from the group consisting of: integrin α6, GDNF, NCAM, GDNF/NCAM complex, macrophages and/or microglia, and combinations thereof such that the systemic and brain parenchymal metastases is prevented and/or treated.

Another aspect of the present disclosure provides a method of preventing and/or treating leptomeningeal disease in a subject, the method comprising, consisting of, or consisting essentially of administering to the subject a therapeutically effective amount of an inhibitory molecule, or a pharmaceutical composition thereof, capable of inhibiting and/or downregulating the binding, signaling, and/or function of a molecule selected from the group consisting of: integrin α6, GDNF, NCAM, GDNF/NCAM complex, macrophages and/or microglia, and combinations thereof such that the leptomeningeal disease is prevented and/or treated.

In some embodiments, the inhibitory molecule is selected from the group consisting of an antibody, oligonucleotide, aptamer, small inhibitory molecule, and combinations thereof.

Suitable anti-integrin α6 antibodies will preferably bind to integrin α6 (the antigen), preferably human integrin α6, and preferable human integrin α6β3l. Other antibodies may be specific to NCAM, preferably human NCAM, antibodies specific to GDNF, preferably human GDNF, and/or antibodies specific for the NCAM/GDNF complex such that signaling is inhibited and/or disrupted. Such antibodies may have a dissociation constant (KD) of one of 1 μM, 100 μM, 10 μM, 1 nM or 100 pM. Binding affinity of an antibody for its target is often described in terms of its dissociation constant (KD). Binding affinity can be measured by methods known in the art, such as by Surface Plasmon Resonance (SPR), or by a radiolabeled antigen binding assay (RIA) performed with the Fab version of the antibody and antigen molecule.

Such antibodies may be antagonist antibodies that inhibit or reduce a biological activity of integrin α6, NCAM, GDNF, and/or the NCAM/GDNF complex. These antibodies may be antagonist antibodies that inhibit or reduce any function of integrin α6, NCAM, GDNF, and/or the NCAM/GDNF complex, in particular signaling.

Such antibodies may be neutralizing antibodies that neutralize the biological effect of integrin α6, NCAM, GDNF, and/or the NCAM/GDNF complex, e.g. the ability to initiate productive signaling mediated by binding of integrin α6, NCAM, GDNF, and/or the NCAM/GDNF complex.

In some embodiments, the antibody is a polyclonal antibody. In other embodiments, the antibody is a monoclonal. In certain embodiments, monoclonal antibody is a humanized monoclonal antibody.

In view of today's techniques in relation to monoclonal antibody technology, antibodies can be prepared to most antigens. The antigen-binding portion may be a part of an antibody (for example a Fab fragment) or a synthetic antibody fragment (for example a single chain Fv fragment [ScFv]). Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimaeric antibodies are discussed by Neuberger et al (1988, 8th International Biotechnology Symposium Part 2, 792-799).

Monoclonal antibodies (mAbs) are useful in the methods of the disclosure and are a homogenous population of antibodies specifically targeting a single epitope on an antigen.

Polyclonal antibodies are useful in the methods of the disclosure. Monospecific polyclonal antibodies are preferred. Suitable polyclonal antibodies can be prepared using methods well known in the art.

Antigen binding fragments of antibodies, such as Fab and Fab2 fragments may also be used/provided as can genetically engineered antibodies and antibody fragments. The variable heavy (VH) and variable light (VL) domains of the antibody are involved in antigen recognition, a fact first recognised by early protease digestion experiments. Further confirmation was found by “humanization” of rodent antibodies. Variable domains of rodent origin may be fused to constant domains of human origin such that the resultant antibody retains the antigenic specificity of the rodent parented antibody (see, e.g., Morrison et al (1984) Proc. Natl. Acad. Sd. USA 81, 6851-6855).

That antigenic specificity is conferred by variable domains and is independent of the constant domains is known from experiments involving the bacterial expression of antibody fragments, all containing one or more variable domains. These molecules include Fab-like molecules (see, e.g., Better et al (1988) Science 240, 1041); Fv molecules (see, e.g., Skerra et al (1988) Science 240, 1038); single-chain Fv (ScFv) molecules where the VH and VL partner domains are linked via a flexible oligopeptide (see, e.g., Bird et al (1988) Science 242, 423; Huston et al (1988) Proc. Natl. Acad. Sd. USA 85, 5879) and single domain antibodies (dAbs) comprising isolated V domains (see, e.g., Ward et al (1989) Nature 341, 544). A general review of the techniques involved in the synthesis of antibody fragments which retain their specific binding sites is to be found in Winter & Milstein (1991) Nature 349, 293-299.

By “ScFv molecules” we mean molecules wherein the VH and VL partner domains are covalently linked, e.g. by a flexible oligopeptide.

Fab, Fv, ScFv and dAb antibody fragments can all be expressed in and secreted from E. coli, thus allowing the facile production of large amounts of the said fragments.

Whole antibodies, and F(ab′)2 fragments are “bivalent”. By “bivalent” we mean that the said antibodies and F(ab′)2 fragments have two antigen combining sites. In contrast, Fab, Fv, ScFv and dAb fragments are monovalent, having only one antigen combining site. Synthetic antibodies which bind to integrin α6, NCAM, GDNF, and/or the NCAM/GDNF complex may also be made using phage display technology as is well known in the art.

Antibodies may be produced by a process of affinity maturation in which a modified antibody is generated that has an improvement in the affinity of the antibody for antigen, compared to an unmodified parent antibody. Affinity-matured antibodies may be produced by procedures known in the art, e.g., Marks et al., Rio/Technology 10:779-783 (1992); Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):331 0-15 9 (1995); and Hawkins et al, J. Mol. Biol. 226:889-896 (1992).

Antibodies according to the present disclosure preferably exhibit specific binding to integrin α6, NCAM, GDNF, and/or the NCAM/GDNF complex. An antibody that specifically binds to a target molecule preferably binds the target with greater affinity, and/or with greater duration than it binds to other targets. In one embodiment, the extent of binding of an antibody to an unrelated target is less than about 10% of the binding of the antibody to the target as measured, e.g., by ELISA, or by a radioimmunoassay (RIA). Alternatively, the binding specificity may be reflected in terms of binding affinity where the antibody binds to integrin α6, NCAM, GDNF, and/or the NCAM/GDNF complex with a KD that is at least 0.1 order of magnitude (i.e. 0.1×10ⁿ, where n is an integer representing the order of magnitude) greater than the KD of the antibody towards another target molecule.

Antibodies may be detectably labelled or, at least, capable of detection. Such antibodies being useful for both in vivo (e.g. imaging methods) and in vitro (e.g. assay methods) applications. For example, the antibody may be labelled with a radioactive atom or a colored molecule or a fluorescent molecule or a molecule which can be readily detected in any other way. Suitable detectable molecules include fluorescent proteins, luciferase, enzyme substrates, and radiolabels. The binding moiety may be directly labelled with a detectable label or it may be indirectly labelled. For example, the binding moiety may be an unlabeled antibody which can be detected by another antibody which is itself labelled. Alternatively, the second antibody may have bound to it biotin and binding of labelled streptavidin to the biotin is used to indirectly label the first antibody.

Aspects of the present disclosure include bi-specific antibodies, e.g. composed of two different fragments of two different antibodies, such that the bi-specific antibody binds two types of antigen. One of the antigens is integrin α6, NCAM, GDNF, and/or the NCAM/GDNF complex, the bi-specific antibody comprising a fragment as described herein that binds to integrin α6, NCAM, GDNF, and/or the NCAM/GDNF complex. The antibody may contain a different fragment having affinity for a second antigen, which may be any desired antigen. Techniques for the preparation of bi-specific antibodies are well known in the art, e.g. see Mueller, D et al., (2010 Biodrugs 24 (2): 89-98), Wozniak-Knopp G et al., (2010 Protein Eng Des 23 (4): 289-297. Baeuerle, P A et al., (2009 Cancer Res 69 (12): 4941-4944).

In some embodiments, the bispecific antibody is provided as a fusion protein of two single-chain variable fragments (scFV) format, comprising a VH and VL of an integrin α6, NCAM, GDNF, and/or the NCAM/GDNF complex binding antibody or antibody fragment, and a VH and VL of an another antibody or antibody fragment.

Bispecific antibodies and bispecific antigen binding fragments may be provided in any suitable format, such as those formats described in Kontermann MAbs 2012, 4(2): 182-197, which is hereby incorporated by reference in its entirety.

Methods for producing bispecific antibodies include chemically crosslinking antibodies or antibody fragments, e.g. with reducible disulphide or non-reducible thioether bonds, for example as described in Segal and Bast, 2001. Production of Bispecific Antibodies. Current Protocols in Immunology. 14:IV:2.13:2.13.1-2.13.16, which is hereby incorporated by reference in its entirety. For example, N-succinimidyl-3-(-2-pyridyldithio)-propionate (SPDP) can be used to chemically crosslink e.g. Fab fragments via hinge region SH— groups, to create disulfide-linked bispecific F(ab)2 heterodimers.

Other methods for producing bispecific antibodies include fusing antibody-producing hybridomas e.g. with polyethylene glycol, to produce a quadroma cell capable of secreting bispecific antibody, for example as described in D. M. and Bast, B. J. 2001. Production of Bispecific Antibodies. Current Protocols in Immunology. 14:IV:2.13:2.13.1-2.13.16.

Bispecific antibodies and bispecific antigen binding fragments can also be produced recombinantly, by expression from e.g. a nucleic acid construct encoding polypeptides for the antigen binding molecules, for example as described in Antibody Engineering: Methods and Protocols, Second Edition (Humana Press, 2012), at Chapter 40: Production of Bispecific Antibodies: Diabodies and Tandem scFv (Hornig and Farber-Schwarz), or French, How to make bispecific antibodies, Methods Mol. Med. 2000; 40:333-339.

For example, a DNA construct encoding the light and heavy chain variable domains for the two antigen binding domains (i.e. the light and heavy chain variable domains for the antigen binding domain capable of binding integrin α6, NCAM, GDNF, and/or the NCAM/GDNF complex, and the light and heavy chain variable domains for the antigen binding domain capable of binding to another target protein), and including sequences encoding a suitable linker or dimerization domain between the antigen binding domains can be prepared by molecular cloning techniques. Recombinant bispecific antibody can thereafter be produced by expression (e.g. in vitro) of the construct in a suitable host cell (e.g. a mammalian host cell), and expressed recombinant bispecific antibody can then optionally be purified.

Peptide or polypeptide based integrin α6, NCAM, GDNF, and/or the NCAM/GDNF complex binding agents may be based on the integrin α6, NCAM, GDNF, and/or the NCAM/GDNF complex proteins or a fragments of integrin α6, NCAM, GDNF, and/or the NCAM/GDNF complex.

In other embodiments an integrin α6, NCAM, GDNF, and/or the NCAM/GDNF complex inhibiting molecule may be provided in the form of a small molecule inhibitor of integrin α6, NCAM, GDNF, and/or the NCAM/GDNF complex.

Peptide or polypeptide based integrin α6, NCAM, GDNF, and/or the NCAM/GDNF complex inhibitory molecules may be based on integrin α6, NCAM, GDNF, and/or the NCAM/GDNF complex, e.g. mutant, variant or binding fragment of integrin α6, NCAM, GDNF, and/or the NCAM/GDNF complex. Suitable peptide or polypeptide-based molecules may bind to integrin α6, NCAM, GDNF, and/or the NCAM/GDNF complex in a manner that does not lead to initiation of signal transduction or produces sub-optimal signaling. In some embodiments an integrin α6, NCAM, GDNF, and/or the NCAM/GDNF complex inhibitory molecule may be provided in the form of a small molecule inhibitor of integrin α6, NCAM, GDNF, and/or the NCAM/GDNF complex.

The inventors have identified that integrin α6, NCAM, GDNF, and/or the NCAM/GDNF complex expression is consistent with the molecular mechanism of metastatic cancer and that inhibition and/or disruption of integrin α6, NCAM, GDNF, and/or the NCAM/GDNF complex activity leads to a reduction in the molecular basis for metastatic cancer. Accordingly, in some aspects of the present disclosure treatment, prevention or alleviation of metastatic cancer may be provided by administration of an inhibitory molecule capable of preventing or reducing the expression of integrin α6, NCAM, GDNF, and/or the NCAM/GDNF complex by cells of the subject.

In some embodiments an inhibitory molecule capable of preventing or reducing the expression of integrin α6, NCAM, GDNF, and/or the NCAM/GDNF complex may be an oligonucleotide capable of repressing or silencing expression of integrin α6, NCAM. GDNF, and/or the NCAM/GDNF complex.

Accordingly, the present disclosure also includes the use of techniques known in the art for the therapeutic down regulation of integrin α6, NCAM, GDNF, and/or the NCAM/GDNF complex expression. These include the use of antisense oligonucleotides and RNA interference (RNAi). As in other aspects of the present disclosure, these techniques may be used in the treatment of metastatic cancer.

Accordingly, in one aspect of the present disclosure a method of treating or preventing metastatic cancer is provided, the method comprising, consisting of, or consisting essentially of administering to a subject in need of treatment a therapeutically effective amount of integrin α6, NCAM, GDNF, and/or the NCAM/GDNF complex inhibiting molecule(s) capable of preventing or reducing the expression of integrin α6, NCAM, GDNF, and/or the NCAM/GDNF complex, wherein the molecule comprises a vector comprising a therapeutic oligonucleotide capable of repressing or silencing expression of integrin α6, NCAM, GDNF, and/or the NCAM/GDNF complex.

In another aspect of the present disclosure a method of treating or preventing metastatic cancer is provided, the method comprising, consisting of, or consisting essentially of administering to a subject in need of treatment a therapeutically effective amount of an agent capable of preventing or reducing the expression of integrin α6, NCAM, GDNF, and/or the NCAM/GDNF complex, wherein the agent comprises an oligonucleotide vector, optionally a viral vector, encoding a therapeutic oligonucleotide capable of being expressed in cells of the subject, the expressed therapeutic oligonucleotide being capable of repressing or silencing expression of integrin α6, NCAM, GDNF, and/or the NCAM/GDNF complex.

The ability of an inhibiting molecule to prevent or reduce the expression of integrin α6, NCAM, GDNF, and/or the NCAM/GDNF complex may be assayed by determining the ability of the agent to inhibit integrin α6, NCAM, GDNF, and/or the NCAM/GDNF complex gene or protein expression by cells.

Reducing the amount of integrin α6, NCAM, GDNF, and/or the NCAM/GDNF complex available for expression provides an alternative means of reducing the level of integrin α6, NCAM, GDNF, and/or the NCAM/GDNF complex-stimulated signaling. Accordingly, in related aspects of the present disclosure, treatment, prevention or alleviation of metastatic cancer may be provided by administration of an inhibitory molecule capable of preventing or reducing the expression of integrin α6, NCAM, GDNF, and/or the NCAM/GDNF complex by cells of the subject.

In some embodiments an inhibiting molecule capable of preventing or reducing the expression of integrin α6, NCAM, GDNF, and/or the NCAM/GDNF complex may be an oligonucleotide capable of repressing or silencing expression of integrin α6, NCAM. GDNF, and/or the NCAM/GDNF complex.

Accordingly, the present disclosure also includes the use of techniques known in the art for the therapeutic down regulation of integrin α6, NCAM, GDNF, and/or the NCAM/GDNF complex expression. These include the use of antisense oligonucleotides and RNA interference (RNAi). As in other aspects of the present disclosure, these techniques may be used in the treatment of metastatic cancer.

Accordingly, one aspect of the present disclosure provides a method of treating or preventing metastatic cancer, the method comprising, consisting of, or consisting essentially of administering to a subject in need of treatment a therapeutically effective amount of an inhibiting molecule capable of preventing or reducing the expression of integrin α6, NCAM, GDNF, and/or the NCAM/GDNF complex, wherein the inhibiting molecule comprises a vector comprising a therapeutic oligonucleotide capable of repressing or silencing expression of integrin α6, NCAM, GDNF, and/or the NCAM/GDNF complex.

In another aspect of the present disclosure a method of treating or preventing metastatic cancer is provided, the method comprising, consisting of, or consisting essentially of administering to a subject in need of treatment a therapeutically effective amount of an inhibiting molecule capable of preventing or reducing the expression of integrin α6, NCAM, GDNF, and/or the NCAM/GDNF complex, wherein the inhibiting molecule comprises an oligonucleotide vector, optionally a viral vector, encoding a therapeutic oligonucleotide capable of being expressed in cells of the subject, the expressed therapeutic oligonucleotide being capable of repressing or silencing expression of integrin α6, NCAM, GDNF, and/or the NCAM/GDNF complex.

The ability of an inhibiting molecule to prevent or reduce the expression of integrin α6, NCAM, GDNF, and/or the NCAM/GDNF complex may be assayed by determining the ability of the agent to inhibit integrin α6, NCAM, GDNF, and/or the NCAM/GDNF complex gene or protein expression by cells.

Aptamers, also called nucleic acid ligands, are nucleic acid molecules characterized by the ability to bind to a target molecule with high specificity and high affinity. Almost every aptamer identified to date is a non-naturally occurring molecule. Aptamers to a given target (e.g. integrin α6, NCAM, GDNF, and/or the NCAM/GDNF complex) may be identified and/or produced, for example, by the method of Systematic Evolution of Ligands by EXponential enrichment (SELEX™). Aptamers and SELEX are described in Tuerk and Gold (Systematic evolution of ligands by exponential enrichment: RNA ligands to bacteriophage T4 DNA polymerase. Science. 1990 Aug. 3; 249(4968):505-10) and in WO91/19813. Aptamers may be DNA or RNA molecules and may be single stranded or double stranded. The aptamer may comprise chemically modified nucleic acids, for example in which the sugar and/or phosphate and/or base is chemically modified. Such modifications may improve the stability of the aptamer or make the aptamer more resistant to degradation and may include modification at the 2′ position of ribose.

Aptamers may be synthesized by methods which are well known to the skilled person. For example, aptamers may be chemically synthesized, e.g. on a solid support. Solid phase synthesis may use phosphoramidite chemistry. Briefly, a solid supported nucleotide is detritylated, then coupled with a suitably activated nucleoside phosphoramidite to form a phosphite triester linkage. Capping may then occur, followed by oxidation of the phosphite triester with an oxidant, typically iodine. The cycle may then be repeated to assemble the aptamer.

Aptamers can be thought of as the nucleic acid equivalent of monoclonal antibodies and often have K_{d}'s in the nM or pM range, e.g. less than one of 500 nM, 100 nM, 50 nM, 10 nM, 1 nM, 500 pM, 100 pM. As with monoclonal antibodies, they may be useful in virtually any situation in which target binding is required, including use in therapeutic and diagnostic applications, in vitro or in vivo. In vitro diagnostic applications may include use in detecting the presence or absence of a target molecule.

Aptamers according to the present disclosure may be provided in purified or isolated form. Aptamers according to the present disclosure may be formulated as a pharmaceutical composition or medicament. Suitable aptamers may optionally have a minimum length of one of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides. Suitable aptamers may optionally have a maximum length of one of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides. Suitable aptamers may optionally have a length of one of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides.

Oligonucleotide molecules, particularly RNA, may be employed to regulate gene expression. These include antisense oligonucleotides, targeted degradation of mRNAs by small interfering RNAs (siRNAs), small molecules, post transcriptional gene silencing (PTGs), developmentally regulated sequence-specific translational repression of mRNA by micro-RNAs (miRNAs) and targeted transcriptional gene silencing.

An antisense oligonucleotide is an oligonucleotide, preferably single stranded, that targets and binds, by complementary sequence binding, to a target oligonucleotide, e.g. mRNA. Where the target oligonucleotide is an mRNA, binding of the antisense to the mRNA blocks translation of the mRNA and expression of the gene product. Antisense oligonucleotides may be designed to bind sense genomic nucleic acid and inhibit transcription of a target nucleotide sequence.

In view of the known nucleic acid sequences for integrin α6, NCAM, GDNF, and/or the NCAM/GDNF complex, oligonucleotides may be designed to repress or silence the expression of integrin α6, NCAM, GDNF, and/or the NCAM/GDNF complex. Such oligonucleotides may have any length. but may preferably be short, e.g. less than 100 nucleotides, e.g. 10-40 nucleotides, or 20-50 nucleotides, and may comprise a nucleotide sequence having complete- or near-complementarity (e.g. 80%, 85%, 90%, 91%, 92%, 93%, 94%, 95%, 96%, 97%, 98%, 99% or 100% complementarity) to a sequence of nucleotides of corresponding length in the target oligonucleotide, e.g. the integrin α6, NCAM, GDNF, and/or the NCAM/GDNF complex mRNA. The complementary region of the nucleotide sequence may have any length, but is preferably at least 5, and optionally no more than 50, nucleotides long, e.g. one of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides.

Repression of integrin α6, NCAM, GDNF, and/or the NCAM/GDNF complex expression will preferably result in a decrease in the quantity of integrin α6, NCAM, GDNF, and/or the NCAM/GDNF complex expressed by a cell. For example, in a given cell the repression of integrin α6, NCAM, GDNF, and/or the NCAM/GDNF complex by administration of a suitable nucleic acid will result in a decrease in the quantity of integrin α6, NCAM, GDNF, and/or the NCAM/GDNF complex expressed by that cell relative to an untreated cell. Repression may be partial. Preferred degrees of repression are at least 50%, more preferably one of at least 60%, 70%, 80%, 85% or 90%. A level of repression between 90% and 100% is considered a ‘silencing’ of expression or function.

A role for the RNAi machinery and small RNAs in targeting of heterochromatin complexes and epigenetic gene silencing at specific chromosomal loci has been demonstrated. Double-stranded RNA (dsRNA)-dependent post transcriptional silencing, also known as RNA interference (RNAi), is a phenomenon in which dsRNA complexes can target specific genes of homology for silencing in a short period of time. It acts as a signal to promote degradation of mRNA with sequence identity. A 20-nt siRNA is generally long enough to induce gene-specific silencing, but short enough to evade host response. The decrease in expression of targeted gene products can be extensive with 90% silencing induced by a few molecules of siRNA. RNAi based therapeutics have been progressed into Phase I, II and III clinical trials for a number of indications (Nature 2009 Jan. 22; 457(7228):426-433).

In the art, these RNA sequences are termed “short or small interfering RNAs” (siRNAs) or “microRNAs” (miRNAs) depending on their origin. Both types of sequence may be used to down-regulate gene expression by binding to complementary RNAs and either triggering mRNA elimination (RNAi) or arresting mRNA translation into protein. siRNAs are derived by processing of long double stranded RNAs and when found in nature are typically of exogenous origin. Micro-interfering RNAs (miRNA) are endogenously encoded small non-coding RNAs, derived by processing of short hairpins. Both siRNA and miRNA can inhibit the translation of mRNAs bearing partially complimentary target sequences without RNA cleavage and degrade mRNAs bearing fully complementary sequences.

Accordingly, the present disclosure provides the use of oligonucleotide sequences for down-regulating the expression of integrin α6, NCAM, GDNF, and/or the NCAM/GDNF complex. siRNA ligands are typically double stranded and, in order to optimize the effectiveness of RNA mediated down-regulation of the function of a target gene, it is preferred that the length of the siRNA molecule is chosen to ensure correct recognition of the siRNA by the RISC complex that mediates the recognition by the siRNA of the mRNA target and so that the siRNA is short enough to reduce a host response.

miRNA ligands are typically single stranded and have regions that are partially complementary enabling the ligands to form a hairpin. miRNAs are RNA genes which are transcribed from DNA, but are not translated into protein. A DNA sequence that codes for a miRNA gene is longer than the miRNA. This DNA sequence includes the miRNA sequence and an approximate reverse complement. When this DNA sequence is transcribed into a single-stranded RNA molecule, the miRNA sequence and its reverse-complement base pair to form a partially double stranded RNA segment. The design of microRNA sequences is discussed in John et al, PLoS Biology, 11(2), 1862-1879, 2004.

Typically, the RNA ligands intended to mimic the effects of siRNA or miRNA have between 10 and 40 ribonucleotides (or synthetic analogues thereof), more preferably between 17 and 30 ribonucleotides, more preferably between 19 and 25 ribonucleotides and most preferably between 21 and 23 ribonucleotides. In some embodiments of the invention employing double-stranded siRNA, the molecule may have symmetric 3′ overhangs, e.g. of one or two (ribo)nucleotides, typically a UU of dTdT 3′ overhang. Based on the disclosure provided herein, the skilled person can readily design suitable siRNA and miRNA sequences, for example using resources such the Ambion siRNA finder. siRNA and miRNA sequences can be synthetically produced and added exogenously to cause gene downregulation or produced using expression systems (e.g. vectors). In a preferred embodiment the siRNA is synthesized synthetically.

Longer double stranded RNAs may be processed in the cell to produce siRNAs (see for example Myers (2003) Nature Biotechnology 21:324-328). The longer dsRNA molecule may have symmetric 3′ or 5′ overhangs, e.g. of one or two (ribo)nucleotides, or may have blunt ends. The longer dsRNA molecules may be 25 nucleotides or longer. Preferably, the longer dsRNA molecules are between 25 and 30 nucleotides long. More preferably, the longer dsRNA molecules are between 25 and 27 nucleotides long. Most preferably, the longer dsRNA molecules are 27 nucleotides in length. dsRNAs 30 nucleotides or more in length may be expressed using the vector pDECAP (Shinagawa et al., Genes and Dev., 17, 1340-5, 2003).

Another alternative is the expression of a short hairpin RNA molecule (shRNA) in the cell. shRNAs are more stable than synthetic siRNAs. A shRNA consists of short inverted repeats separated by a small loop sequence. One inverted repeat is complimentary to the gene target. In the cell the shRNA is processed by DICER into a siRNA which degrades the target gene mRNA and suppresses expression. In a preferred embodiment the shRNA is produced endogenously (within a cell) by transcription from a vector. shRNAs may be produced within a cell by transfecting the cell with a vector encoding the shRNA sequence under control of a RNA polymerase III promoter such as the human H1 or 7SK promoter or a RNA polymerase II promoter. Alternatively, the shRNA may be synthesised exogenously (in vitro) by transcription from a vector. The shRNA may then be introduced directly into the cell. Preferably, the shRNA molecule comprises a partial sequence of integrin α6, NCAM, GDNF, and/or the NCAM/GDNF complex. Preferably, the shRNA sequence is between 40 and 100 bases in length, more preferably between 40 and 70 bases in length. The stem of the hairpin is preferably between 19 and 30 base pairs in length. The stem may contain G-U pairings to stabilize the hairpin structure. siRNA molecules, longer dsRNA molecules or miRNA molecules may be made recombinantly by transcription of a nucleic acid sequence, preferably contained within a vector. Preferably, the siRNA molecule, longer dsRNA molecule or miRNA molecule comprises a partial sequence of integrin α6, NCAM, GDNF, and/or the NCAM/GDNF complex.

In one embodiment, the siRNA, longer dsRNA or miRNA is produced endogenously (within a cell) by transcription from a vector. The vector may be introduced into the cell in any of the ways known in the art. Optionally, expression of the RNA sequence can be regulated using a tissue specific (e.g. heart, liver, kidney or eye specific) promoter. In a further embodiment, the siRNA, longer dsRNA or miRNA is produced exogenously (in vitro) by transcription from a vector. Suitable vectors may be oligonucleotide vectors configured to express the oligonucleotide agent capable of integrin α6, NCAM, GDNF, and/or the NCAM/GDNF complex repression. Such vectors may be viral vectors or plasmid vectors.

The therapeutic oligonucleotide may be incorporated in the genome of a viral vector and be operably linked to a regulatory sequence, e.g. promoter, which drives its expression. The term “operably linked” may include the situation where a selected nucleotide sequence and regulatory nucleotide sequence are covalently linked in such a way as to place the expression of a nucleotide sequence under the influence or control of the regulatory sequence. Thus a regulatory sequence is operably linked to a selected nucleotide sequence if the regulatory sequence is capable of effecting transcription of a nucleotide sequence which forms part or all of the selected nucleotide sequence.

Viral vectors encoding promoter-expressed siRNA sequences are known in the art and have the benefit of long term expression of the therapeutic oligonucleotide. Examples include lentiviral (Nature 2009 Jan. 22; 457(7228):426-433), adenovirus (Shen et al., FEBS Lett 2003 Mar. 27; 539(1-3)111-4) and retroviruses (Barton and Medzhitov PNAS Nov. 12, 2002 vol. 99, no. 23 14943-14945).

In other embodiments a vector may be configured to assist delivery of the therapeutic oligonucleotide to the site at which repression of integrin α6, NCAM, GDNF, and/or the NCAM/GDNF complex expression is required. Such vectors typically involve complexing the oligonucleotide with a positively charged vector (e.g., cationic cell penetrating peptides, cationic polymers and dendrimers, and cationic lipids); conjugating the oligonucleotide with small molecules (e.g., cholesterol, bile acids, and lipids), polymers, antibodies, and RNAs; or encapsulating the oligonucleotide in nanoparticulate formulations (Wang et al., AAPS J. 2010 December; 12(4): 492-503).

In one embodiment, a vector may comprise a nucleic acid sequence in both the sense and antisense orientation, such that when expressed as RNA the sense and antisense sections will associate to form a double stranded RNA.

Alternatively, siRNA molecules may be synthesized using standard solid or solution phase synthesis techniques which are known in the art. Linkages between nucleotides may be phosphodiester bonds or alternatives, for example, linking groups of the formula P(O)S, (thioate); P(S)S, (dithioate); P(O)NR′2; P(O)R′; P(O)OR⁶; CO; or CONR′2 wherein R is H (or a salt) or alkyl (1-12C) and R⁶ is alkyl (1-9C) is joined to adjacent nucleotides through —O— or —S—.

Modified nucleotide bases can be used in addition to the naturally occurring bases, and may confer advantageous properties on siRNA molecules containing them. For example, modified bases may increase the stability of the siRNA molecule, thereby reducing the amount required for silencing. The provision of modified bases may also provide siRNA molecules which are more, or less, stable than unmodified siRNA. The term ‘modified nucleotide base’ encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Thus modified nucleotides may also include 2′ substituted sugars such as 2′-O-methyl-; 2′-O-alkyl; 2′-O-allyl; 2′-S-alkyl; 2′-S-allyl; 2′-fluoro-; 2′-halo or azido-ribose, carbocyclic sugar analogues, a-anomeric sugars; epimeric sugars such as arabinose, xyloses or lyxoses, pyranose sugars, furanose sugars, and sedoheptulose.

Modified nucleotides are known in the art and include alkylated purines and pyrimidines, acylated purines and pyrimidines, and other heterocycles. These classes of pyrimidines and purines are known in the art and include pseudoisocytosine, N4,N4-ethanocytosine, 8-hydroxy-N6-methyladenine, 4-acetylcytosine, 5-(carboxyhydroxylmethyl) uracil, 5 fluorouracil, 5-bromouracil, 5-carboxymethylaminomethyl-2-thiouracil, 5-carboxymethylaminomethyl uracil, dihydrouracil, inosine, N6-isopentyl-adenine, 1-methyladenine, 1-methylpseudouracil, 1-methylguanine, 2,2-dimethylguanine, 2-methyladenine, 2-methylguanine, 3-methylcytosine, 5-methylcytosine, N6-methyladenine, 7-methylguanine, 5-methylaminomethyl uracil, 5-methoxy amino methyl-2-thiouracil, -D-mannosylqueosine, 5-methoxycarbonylmethyluracil, 5methoxyuracil, 2 methylthio-N6-isopentenyladenine, uracil-5-oxyacetic acid methyl ester, psueouracil, 2-thiocytosine, 5-methyl-2 thiouracil, 2-thiouracil, 4-thiouracil, 5methyluracil, N-uracil-5-oxy acetic acid methylester, uracil 5-oxyacetic acid, queosine, 2-thiocytosine, 5-propyluracil, 5-propylcytosine, 5-ethyluracil, 5ethylcytosine, 5-butyluracil, 5-pentyluracil, 5-pentylcytosine, and 2,6,diaminopurine, methylpsuedouracil, 1-methylguanine, 1-methylcytosine.

Methods relating to the use of RNAi to silence genes in C. elegans, Drosophila, plants, and mammals are known in the art (Fire A, et al., 1998 Nature 391:806-811; Fire, A. Trends Genet. 15, 358-363 (1999); Sharp, P. A. RNA interference 2001. Genes Dev. 15, 485-490 (2001); Hammond, S. M., et al., Nature Rev. Genet. 2, 110-1119 (2001); Tuschl, T. Chem. Biochem. 2, 239-245 (2001); Hamilton, A. et al., Science 286, 950-952 (1999); Hammond, S. M., et al., Nature 404, 293-296 (2000); Zamore, P. D., et al., Cell 101, 25-33 (2000); Bernstein, E., et al., Nature 409, 363-366 (2001); Elbashir, S. M., et al., Genes Dev. 15, 188-200 (2001); WO0129058; WO9932619, and Elbashir S M, et al., 2001 Nature 411:494-498).

Accordingly, the present disclosure provides a nucleic acid that is capable, when suitably introduced into or expressed within a mammalian, e.g. human, cell that otherwise expresses integrin α6, NCAM, GDNF, and/or the NCAM/GDNF complex, of suppressing integrin α6, NCAM, GDNF, and/or the NCAM/GDNF complex expression by RNAi. The nucleic acid may have substantial sequence identity to a portion of integrin α6, NCAM, GDNF, and/or the NCAM/GDNF complex mRNA, or the complementary sequence to said mRNA.

The nucleic acid may be a double-stranded siRNA. (As the skilled person will appreciate, and as explained further below, a siRNA molecule may include a short 3′ DNA sequence also.) Alternatively, the nucleic acid may be a DNA (usually double-stranded DNA) which, when transcribed in a mammalian cell, yields an RNA having two complementary portions joined via a spacer, such that the RNA takes the form of a hairpin when the complementary portions hybridize with each other. In a mammalian cell, the hairpin structure may be cleaved from the molecule by the enzyme DICER, to yield two distinct, but hybridized, RNA molecules. Only single-stranded (i.e. non self-hybridized) regions of an mRNA transcript are expected to be suitable targets for RNAi. It is therefore proposed that other sequences very close in the integrin α6, NCAM, GDNF, and/or the NCAM/GDNF complex mRNA transcript may also be suitable targets for RNAi.

Accordingly, the present disclosure provides nucleic acids that are capable, when suitably introduced into or expressed within a mammalian cell that otherwise expresses integrin α6, NCAM, GDNF, and/or the NCAM/GDNF complex, of suppressing integrin α6, NCAM, GDNF, and/or the NCAM/GDNF complex expression by RNAi, wherein the nucleic acid is generally targeted to the sequence of, or portion thereof, of integrin α6, NCAM, GDNF, and/or the NCAM/GDNF complex. By “generally targeted” the nucleic acid may target a sequence that overlaps with integrin α6, NCAM, GDNF, and/or the NCAM/GDNF complex. In particular, the nucleic acid may target a sequence in the mRNA of human integrin α6, NCAM, GDNF, and/or the NCAM/GDNF complex that is slightly longer or shorter than one of integrin α6, NCAM, GDNF, and/or the NCAM/GDNF complex, but is otherwise identical to the native form.

It is expected that perfect identity/complementarity between the nucleic acid of the invention and the target sequence, although preferred, is not essential. Accordingly, the nucleic acid of the present disclosure may include a single mismatch compared to the mRNA of integrin α6, NCAM, GDNF, and/or the NCAM/GDNF complex. It is expected, however, that the presence of even a single mismatch is likely to lead to reduced efficiency, so the absence of mismatches is preferred. When present, 3′ overhangs may be excluded from the consideration of the number of mismatches.

The term “complementarity” is not limited to conventional base pairing between nucleic acid consisting of naturally occurring ribo- and/or deoxyribonucleotides, but also includes base pairing between mRNA and nucleic acids of the invention that include non-natural nucleotides.

In one embodiment, the nucleic acid (herein referred to as double-stranded siRNA) includes the double-stranded RNA sequences for integrin α6, NCAM, GDNF, and/or the NCAM/GDNF complex. However, it is also expected that slightly shorter or longer sequences directed to the same region of integrin α6, NCAM. GDNF, and/or the NCAM/GDNF complex mRNA will also be effective. In particular, it is expected that double-stranded sequences between 17 and 23 bp in length will also be effective.

The strands that form the double-stranded RNA may have short 3′ dinucleotide overhangs, which may be DNA or RNA. The use of a 3′ DNA overhang has no effect on siRNA activity compared to a 3′ RNA overhang, but reduces the cost of chemical synthesis of the nucleic acid strands (Elbashir et al., 2001c). For this reason, DNA dinucleotides may be preferred. When present, the dinucleotide overhangs may be symmetrical to each other, though this is not essential. Indeed, the 3′ overhang of the sense (upper) strand is irrelevant for RNAi activity, as it does not participate in mRNA recognition and degradation (Elbashir et al., 2001a, 2001b, 2001c). While RNAi experiments in Drosophila show that antisense 3′ overhangs may participate in mRNA recognition and targeting (Elbashir et al. 2001c), 3′ overhangs do not appear to be necessary for RNAi activity of siRNA in mammalian cells. Incorrect annealing of 3′ overhangs is therefore thought to have little effect in mammalian cells (Elbashir et al. 2001c; Czaudema et al. 2003).

Any dinucleotide overhang may therefore be used in the antisense strand of the siRNA. Nevertheless, the dinucleotide is preferably -UU or -UG (or -TT or -TG if the overhang is DNA), more preferably -UU (or -TT). The -UU (or -TT) dinucleotide overhang is most effective and is consistent with (i.e. capable of forming part of) the RNA polymerase III end of transcription signal (the terminator signal is TTTTT). Accordingly, this dinucleotide is most preferred. The dinucleotides AA, CC and GG may also be used, but are less effective and consequently less preferred. Moreover, the 3′ overhangs may be omitted entirely from the siRNA.

The present disclosure also provides single-stranded nucleic acids (herein referred to as single-stranded siRNAs) respectively consisting of a component strand of one of the aforementioned double-stranded nucleic acids, preferably with the 3′-overhangs, but optionally without. The present disclosure also provides kits containing pairs of such single-stranded nucleic acids, which are capable of hybridizing with each other in vitro to form the aforementioned double-stranded siRNAs, which may then be introduced into cells.

The present disclosure also provides DNA that, when transcribed in a mammalian cell, yields an RNA (herein also referred to as an shRNA) having two complementary portions which are capable of self-hybridizing to produce a double-stranded motif or a sequence that differs from any one of the aforementioned sequences by a single base pair substitution.

The complementary portions will generally be joined by a spacer, which has suitable length and sequence to allow the two complementary portions to hybridize with each other. The two complementary (i.e. sense and antisense) portions may be joined 5′-3′ in either order. The spacer will typically be a short sequence, of approximately 4-12 nucleotides, preferably 4-9 nucleotides, more preferably 6-9 nucleotides.

Preferably the 5′ end of the spacer (immediately 3′ of the upstream complementary portion) consists of the nucleotides -UU- or -UG-, again preferably -UU- (though, again, the use of these particular dinucleotides is not essential). A suitable spacer, recommended for use in the pSuper system of OligoEngine (Seattle, Wash., USA) is UUCAAGAGA (SEQ ID NO: 1). In this and other cases, the ends of the spacer may hybridize with each other.

Similarly, the transcribed RNA preferably includes a 3′ overhang from the downstream complementary portion. Again, this is preferably —UU or -UG, more preferably -UU. Such shRNA molecules may then be cleaved in the mammalian cell by the enzyme DICER to yield a double-stranded siRNA as described above, in which one or each strand of the hybridized dsRNA includes a 3′ overhang. Techniques for the synthesis of the nucleic acids of the invention are of course well known in the art. The skilled person is well able to construct suitable transcription vectors for the DNA of the present disclosure using well-known techniques and commercially available materials. In particular, the DNA will be associated with control sequences, including a promoter and a transcription termination sequence. Of particular suitability are the commercially available pSuper and pSuperior systems of OligoEngine (Seattle, Wash., USA). These use a polymerase-III promoter (H1) and a T5 transcription terminator sequence that contributes two U residues at the 3′ end of the transcript (which, after DICER processing, provide a 3′ UU overhang of one strand of the siRNA). Another suitable system is described in Shin et al. (RNA, 2009 May; 15(5): 898-910), which uses another polymerase-III promoter (U6). The double-stranded siRNAs of the present disclosure may be introduced into mammalian cells in vitro or in vivo using known techniques, as described below, to suppress expression of integrin α6, NCAM, GDNF, and/or the NCAM/GDNF complex. Similarly, transcription vectors containing the DNAs of the present disclosure may be introduced into tumor cells in vitro or in vivo using known techniques, as described below, for transient or stable expression of RNA, again to suppress expression of integrin α6, NCAM, GDNF, and/or the NCAM/GDNF complex.

Accordingly, the present disclosure also provides a method of suppressing integrin α6, NCAM, GDNF, and/or the NCAM/GDNF complex expression in a mammalian, e.g. human, cell, the method comprising administering to the cell a double-stranded siRNA of the present disclosure or a transcription vector of the present disclosure. Similarly, the present disclosure further provides a method of treating metastatic cancer, the method comprising administering to a subject a double-stranded siRNA of the invention or a transcription vector of the present disclosure.

The present disclosure further provides the double-stranded siRNAs of the present disclosure and the transcription vectors of the invention, for use in a method of treatment, preferably a method of treating metastatic cancer.

The present disclosure further provides the use of the double-stranded siRNAs of the present disclosure and the transcription vectors of the disclosure in the preparation of a medicament for the treatment of metastatic cancer.

The present disclosure further provides a composition comprising a double-stranded siRNA of the present disclosure or a transcription vector of the disclosure in admixture with one or more pharmaceutically acceptable carriers. Suitable carriers include lipophilic carriers or vesicles, which may assist in penetration of the cell membrane.

Materials and methods suitable for the administration of siRNA duplexes and DNA vectors of the present disclosure are well known in the art and improved methods are under development, given the potential of RNAi technology.

Generally, many techniques are available for introducing nucleic acids into mammalian cells. The choice of technique will depend on whether the nucleic acid is transferred into cultured cells in vitro or in vivo in the cells of a patient. Techniques suitable for the transfer of nucleic acid into mammalian cells in vitro include the use of liposomes, electroporation, microinjection, cell fusion, DEAE dextran and calcium phosphate precipitation. In vivo gene transfer techniques include transfection with viral (typically retroviral) vectors and viral coat protein-liposome mediated transfection (Dzau et al. (2003) Trends in Biotechnology 11, 205-210). In particular, suitable techniques for cellular administration of the nucleic acids of the present disclosure both in vitro and in vivo are disclosed in the following articles: General reviews: Borkhardt, A. 2002. Blocking oncogenes in malignant cells by RNA interference—new hope for a highly specific cancer treatment?Cancer Cell. 2:167-8. Hannon, G. J. 2002. RNA interference. Nature. 418:244-51. McManus, M. T., and P. A. Sharp. 2002. Gene silencing in mammals by small interfering RNAs. Nat Rev Genet. 3:737-47. Scherr, M., M. A. Morgan, and M. Eder. 2003b. Gene silencing mediated by small interfering RNAs in mammalian cells. Curr Med Chem. 10:245-56. Shuey, D. J., D. E. McCallus, and T. Giordano. 2002. RNAi: gene-silencing in therapeutic intervention. Drug Discov Today. 7:1040-6.

Systemic delivery using liposomes: Lewis, D. L., J. E. Hagstrom, A. G. Loomis, J. A. Wolff, and H. Herweijer. 2002. Efficient delivery of siRNA for inhibition of gene expression in postnatal mice. Nat Genet. 32:107-8. Paul, C. P., P. D. Good, I. Winer, and D. R. Engelke. 2002. Effective expression of small interfering RNA in human cells. Nat Biotechnol. 20:505-8. Song, E., S. K. Lee, J. Wang, N. Ince, N. Ouyang, J. Min, J. Chen, P. Shankar, and J. Lieberman. 2003. RNA interference targeting Fas protects mice from fulminant hepatitis. Nat Med. 9:347-51. Sorensen, D. R., M. Leirdal, and M. Sioud. 2003. Gene silencing by systemic delivery of synthetic siRNAs in adult mice. J Mol Biol. 327:761-6.

Virus mediated transfer: Abbas-Terki, T., W. Blanco-Bose, N. Deglon, W. Pralong, and P. Aebischer. 2002. Lentiviral-mediated RNA interference. Hum Gene Ther. 13:2197-201. Barton, G. M., and R. Medzhitov. 2002. Retroviral delivery of small interfering RNA into primary cells. Proc Natl Acad Sci USA. 99:14943-5. Devroe, E., and P. A. Silver. 2002. Retrovirus-delivered siRNA. BMC Biotechnol. 2:15. Lori, F., P. Guallini, L. Galluzzi, and J. Lisziewicz. 2002. Gene therapy approaches to HIV infection. Am J Pharmacogenomics. 2:245-52. Matta, H., B. Hozayev, R. Tomar, P. Chugh, and P. M. Chaudhary. 2003. Use of lentiviral vectors for delivery of small interfering RNA. Cancer Biol Ther. 2:206-10. Qin, X. F., D. S. An, I. S. Chen, and D. Baltimore. 2003. Inhibiting HIV-1 infection in human T cells by lentiviral-mediated delivery of small interfering RNA against CCR5. Proc Natl Acad Sci USA. 100:183-8. Scherr, M., K. Battmer, A. Ganser, and M. Eder. 2003a. Modulation of gene expression by lentiviral-mediated delivery of small interfering RNA. Cell Cycle. 2:251-7. Shen, C., A. K. Buck, X. Liu, M. Winkler, and S. N. Reske. 2003. Gene silencing by adenovirus-delivered siRNA. FEBS Lett. 539:111-4.

Peptide delivery: Morris, M. C., L. Chaloin, F. Heitz, and G. Divita. 2000. Translocating peptides and proteins and their use for gene delivery. Curr Opin Biotechnol. 11:461-6. Simeoni, F., M. C. Morris, F. Heitz. and G. Divita. 2003. Insight into the mechanism of the peptide-based gene delivery system MPG: implications for delivery of siRNA into mammalian cells. Nucleic Acids Res. 31:2717-24. Other technologies that may be suitable for delivery of siRNA to the target cells are based on nanoparticles or nanocapsules such as those described in U.S. Pat. Nos. 6,649,192B and 5,843,509B.

Another aspect of the present disclosure provides for the use of gene therapy, such as that provided by CRISPR gene therapy, to the prevention and treatment of metastatic cancer.

Accordingly, one aspect of the present disclosure provides a polynucleotide for the deletion of a gene encoding integrin α6, NCAM, GDNF, and/or the NCAM/GDNF complex, comprising, consisting of, or consisting essentially of a first promoter operably linked to a guide RNA (e.g., sgRNA) and optionally a TracrRNA, and a second promoter operably linked to, for example, SaCas9.

The “guide sequence portion” or “sequence guide portion” of an RNA molecule are used interchangeably (referred to as sgRNA herein) and refers to a nucleotide sequence that is capable of hybridizing to a specific target DNA sequence, e.g., the guide sequence portion has a nucleotide sequence which is fully complementary to the DNA sequence being targeted along the length of the guide sequence portion. In some embodiments, the guide sequence portion is 17, 18, 19, 20, 21, 22, 23, 24, 25, 26, 27, 28, 29, or 30 nucleotides in length, or approximately 17-30, 17-29, 17-28, 17-27, 17-26, 17-25, 17-24, 18-22, 19-22, 18-20, 17-20, or 17-22 nucleotides in length. The entire length of the guide sequence portion is fully complementary to the DNA sequence being targeted along the length of the guide sequence portion. The guide sequence portion may be part of an RNA molecule that can form a complex with a CRISPR nuclease with the guide sequence portion serving as the DNA targeting portion of the CRISPR complex. When the DNA molecule having the guide sequence portion is present contemporaneously with the CRISPR molecule the RNA molecule is capable of targeting the CRISPR nuclease to the specific target DNA sequence. Each possibility represents a separate embodiment. An RNA molecule can be custom designed to target any desired sequence. In one embodiment, the sgRNA comprises a sgRNA associated with integrin α6, NCAM, GDNF, and/or the NCAM/GDNF complex.

The polypeptides according to the present disclosure may comprise at least two promoters operably linked to an sgRNA or Sa Cas9. “Promoter” as used herein means a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. In one embodiment, the first promoter comprises an RNA polymerase promoter. In certain embodiments, the RNA polymerase promoter comprises a U6 promoter. In another embodiment, the second promoter comprises a tissue-specific promoter.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

Another aspect comprises a DNA targeting system comprising, consisting of, or consisting essentially of a first polynucleotide, the first polynucleotide comprising, consisting of, or consisting essentially of a first promoter operably linked to an sgRNA associated with integrin α6, NCAM, GDNF, and/or the NCAM/GDNF complex and a TracrRNA and a second promoter operably linked to SaCas9 and the second polynucleotide comprising, consisting of, or consisting essentially of a first promoter operably linked to an sgRNA associated with integrin α6, NCAM, GDNF, and/or the NCAM/GDNF complex and a TracrRNA and a second promoter operably linked to SaCas9.

Another aspect of the present disclosure provides a vector comprising, consisting of, or consisting essentially of one or more polynucleotides as provided herein. In one embodiment, the vector comprises a recombinant viral vector. In one embodiment, the recombinant viral vector comprises an AAV vector.

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAV type rh32.33, AAV type rh8, AAV type rh10, avian AAV, bovine AAV, canine AAV, equine AAV, ovine AAV, and any other AAV now known or later discovered. See, e.g., BERNARD N. FIELDS et al., VIROLOGY, volume 2, chapter 69 (4th ed., Lippincott-Raven Publishers). A number of AAV serotypes and clades have been identified (see, e.g., Gao et al, (2004) J. Virology 78:6381-6388; Moris et al, (2004) Virology 33-:375-383; and Table 1).

The genomic sequences of various serotypes of AAV and the autonomous parvoviruses, as well as the sequences of the native terminal repeats (TRs), Rep proteins, and capsid subunits are known in the art. Such sequences may be found in the literature or in public databases such as GenBank. See, e.g., GenBank Accession Numbers NC_002077, NC_001401, NC 001729, NC 001863, NC_001829, NC 001862, NC 000883, NC_001701, NC 001510, NC 006152, NC_006261, AF063497, U89790, AF043303, AF028705, AF028704, J02275, J01901, J02275, X01457, AF288061, AH009962, AY028226, AY028223, NC 001358, NC_001540, AF513851, AFS13852, AY530579; the disclosures of which are incorporated by reference herein for teaching parvovirus and AAV nucleic acid and amino acid sequences.

In the context of the present invention, the term “reference” relates to a standard in relation to quantity, quality or type, against which other values or characteristics can be compared, such as a standard curve.

In the present invention, the reference values may be the expression and/or activity levels of one or more biomarkers (e.g., integrin α6, BCCs expressing integrin α6. GDNF, NRTN, ARTN, and PSPN). A set of reference data may be established by collecting the reference values for a number of samples. As will be obvious to those of skill in the art, the set of reference data will improve by including increasing numbers of reference values.

In some embodiments, the reference means is an internal reference means and/or an external reference means. In the present context the term “internal reference means” relates to a reference which is not handled by the user directly for each determination, but which is incorporated into a device for the determination of the concentration/level of one or more biomarkers (e.g., integrin α6, BCCs expressing integrin α6, GDNF, NRTN, ARTN, and PSPN), whereby only the ‘final result’ or the ‘final measurement’ is presented. The terms the “final result” or the “final measurement” relate to the result presented to the user when the reference value has been taken into account. In the present context, the term “external reference means” relates to a reference which is handled directly by the user in order to determine the concentration/level of one or more of biomarkers (e.g., integrin α6, BCCs expressing integrin α6, GDNF, NRTN, ARTN, and PSPN), before obtaining the “final result”. In yet a further embodiment of the present invention external reference means are selected from the group consisting of a table, a diagram and similar reference means where the user can compare the measured signal to the selected reference means. To determine whether the subject has an biomarker level over-expressing subtype, a cut-off must be established. This cut-off may be established by the laboratory, the physician or on a case-by-case basis for each subject. The cut-off level could be established using a number of methods, including: percentiles, mean plus or minus standard deviation(s); multiples of median value; patient specific risk or other methods known to those skilled in the art.

The multivariate discriminant analysis and other risk assessments can be performed on the commercially available computer program statistical package Statistical Analysis System (manufactured and sold by SAS Institute Inc.) or by other methods of multivariate statistical analysis or other statistical software packages or screening software known to those skilled in the art.

As obvious to one skilled in the art, in any of the embodiments discussed above, changing the cut-off level could change the results of the discriminant analysis for each patient.

When expression and/or activity levels of a biomarker are compared to a reference level, they can either be different (above or below the reference value) or equal. However, using today's detection techniques an exact definition of different or equal result can be difficult because of noise and variations in obtained expression levels from different samples. Hence, the usual method for evaluating whether two or more expression levels are different or equal involves statistical analysis.

Statistical analysis enables evaluation of significantly different expression levels and significantly equal expressions levels. Statistical methods involve applying a function/statistical algorithm to a set of data. Statistical theory defines a statistic as a function of a sample where the function itself is independent of the sample's distribution: the term is used both for the function and for the value of the function on a given sample. Commonly used statistical tests or methods applied to a data set include t-test, f-test or even more advanced tests and methods of comparing data. Using such tests or methods enables a conclusion of whether two or more samples are significantly different or significantly equal.

The significance may be determined by the standard statistical methodology known by the person skilled in the art. The chosen reference level may be changed depending on the mammal for which the test is applied. The chosen reference level may be changed if desiring a different specificity or sensitivity as known in the art.

As used herein the sensitivity refers to the measures of the proportion of actual positives, which are correctly identified as such—in analogy with a diagnostic test, i.e. the percentage of mammals or people overexpressing the one or more of biomarkers (e.g., integrin α6, BCCs expressing integrin α6, GDNF, NRTN, ARTN, and PSPN). Usually the sensitivity of a test can be described as the proportion of true positives of the total number. As used herein the specificity refers to measures of the proportion of negatives, which are correctly identified—i.e. the percentage of mammal with an biomarker level equal to or below normal. The ideal diagnostic test is a test that has 100% specificity, i.e. only detects mammals which over-express the biomarker and, therefore, no false positive results, and has 100% sensitivity.

For any test, there is usually a trade-off between each measure. For example in a manufacturing setting in which one is testing for faults, one may be willing to risk discarding functioning components (low specificity), in order to increase the chance of identifying nearly all faulty components (high sensitivity). This trade-off can be represented graphically using a receiver operating characteristic (ROC curve).

Thus, in an embodiment the subtypes according to the present invention has an upregulated level (over-expression) of a biomarker of at least 2×, such as at least 4×, such as at least 6×, such as at least 10× compared to reference tissue.

In some embodiments, the reference level is an expression and/or activity level of one or more of integrin α6, BCCs expressing integrin α6, GDNF, NRTN, ARTN, and PSPN correlated with a low risk or no risk of developing LMD. In some embodiments, the reference level is an expression and/or activity level of one or more of integrin α6, BCCs expressing integrin α6, GDNF, NRTN, ARTN, and PSPN correlated with a high risk of developing LMD.

The present disclosure further provides kits comprising the compositions provided herein and for carrying out the subject methods as provided herein. For example, in one embodiment, a subject kit may comprise, consist of, or consist essentially of one or more of the following: (i) an inhibitory molecule as provided herein; (ii) a cell system incorporation an inhibitory molecule and/or for gene deletion (e.g., CRISPR) as provided herein; (iii) delivery systems comprising an inhibitory molecule as provided herein; (iv) cells comprising a vector encoding an inhibitory molecule as provided herein or cell system as provided herein; and/or (v) pharmaceutical compositions as provided herein.

In other embodiments, a kit may further include other components. Such components may be provided individually or in combinations, and may provide in any suitable container such as a vial, a bottle, or a tube. Examples of such components include, but are not limited to, (i) one or more additional reagents, such as one or more dilution buffers; one or more reconstitution solutions; one or more wash buffers; one or more storage buffers, one or more control reagents and the like, (ii) one or more control expression vectors or RNA polynucleotides; (iii) one or more reagents for in vitro production and/or maintenance of the of the molecules, cells, delivery systems etc. provided herein; and the like. Components (e.g., reagents) may also be provided in a form that is usable in a particular assay, or in a form that requires addition of one or more other components before use (e.g. in concentrate or lyophilized form). Suitable buffers include, but are not limited to, phosphate buffered saline, sodium carbonate buffer, sodium bicarbonate buffer, borate buffer, Tris buffer, MOPS buffer, HEPES buffer, and combinations thereof.

In addition to above-mentioned components, a subject kit can further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

Another aspect of the present disclosure provides all that is described and illustrated herein.

### EXAMPLES

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention. The use of pronouns such as “I”, “we”, and “our”, for example, refer to the inventors.

### Example I

This example demonstrates that BCCs enter the CNS along emissary vessels bridging BM and LM.

Our group recently discovered that leukemia cells in the vertebral/calvarial bone marrow (BM) can invade the LM by migrating along the laminin-rich abluminal/external surface of emissary vessels that directly connect the BM and LM, bypassing the need to cross the blood-brain barrier (FIG. 1a)¹⁰. Subsequent work has revealed that benign immune cells also utilize this transit corridor to efficiently enter the meninges.¹¹⁻¹⁴. Autopsy analysis has shown that the majority of patients with BC LM metastasis have coexisting, sometimes occult, vertebral or calvarial bone metastases⁹. We therefore utilized xenograft and syngeneic mouse models of bone metastatic BC to investigate whether a BM-LM corridor could be employed by malignant epithelial cells to colonize the LM.

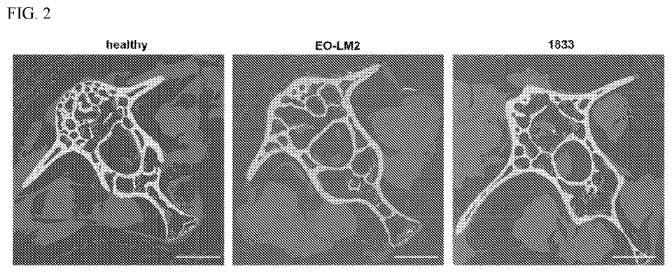

Overt LMD involvement is an infrequent occurrence in systemically-engrafted mouse models of metastatic BC¹⁵. In the bone tropic 1833-tdTomato-Parental (1833-P) subclone model of the human MDA-MB-231 cell line, however, we found that hindlimb paralysis (HLP) is a common sequela of lumbar LM metastases and cauda equina syndrome (FIG. 1b,c). Spontaneous LM metastasis is also rare in syngeneic mouse BC models. Through serial intracardiac engraftment of murine E0771-tdTomato-Parental (EO-P) BC cells harvested from the meninges of a mouse with LM metastasis, we generated a subclone after two passages (EO-LM2) that displays a 100% rate of HLP due to LMD (FIG. 1d-f and Table 2). Both the 1833-P xenograft model and EO-LM2 syngeneic model exhibit frequent BM metastases, including within the vertebrae and calvarium. MicroCT imaging of the lumbosacral vertebrae confirmed the integrity of the spinal canal, excluding vertebral bone fractures as a cause of spinal cord compression and HLP (FIG. 2b).

TABLE 2
Mouse HLP Scoring Guide

| Score | Clinical observations |
|---|---|
| 0 | No changes in motor function. |
| 0.5 | Tip of the tail is limp but muscle straining is felt. |
| 1 | Entire tail is limp. When picked up by the base, the tail |
| | drapes over the finger. |
| 1.5 | Limp fail and weakness in one hind limb. Walking may be |
| | slightly wobbly. |
| 2 | Limp tail and weakness in hind limits, Walking is wobbly. |
| | One hind limb may be dragging while the other is weak. |
| 2.5 | Limp tail and hind limb paresis. The hind limbs are both |
| | dragging but have some movement. - OR - One hind limb is |
| | paralyzed and the other is weak or dragging but with |
| | some movement. |
| 3 | Limp tail and bilateral hind limb paralysis. |

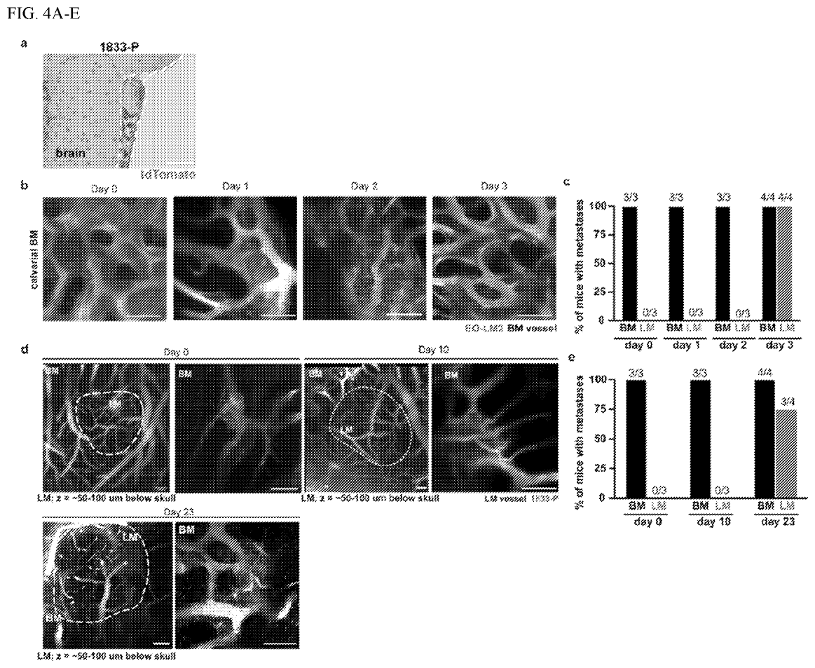

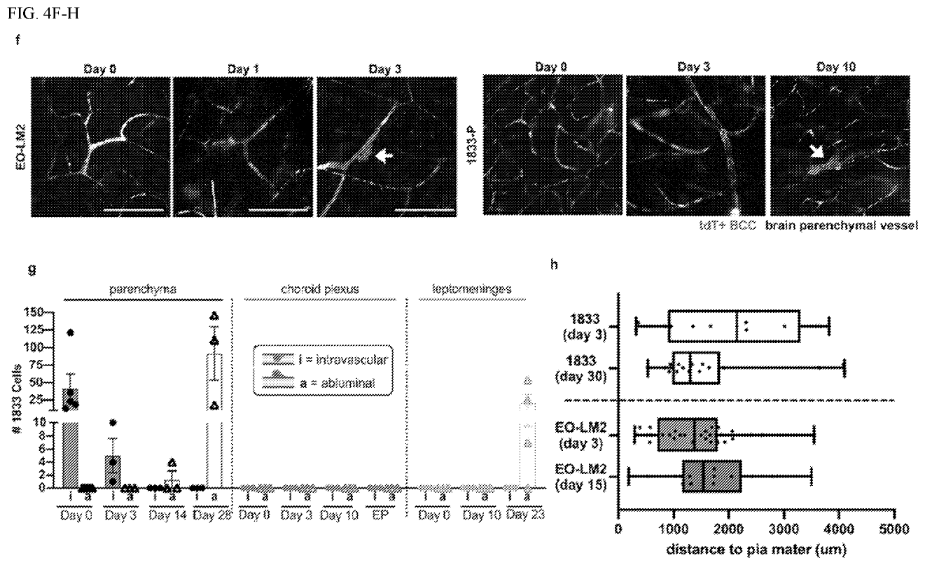

To characterize potential routes of LM metastasis in these models (FIG. 3a), we used a combination of ex vivo and real-time in vivo approaches to analyze BCC interactions with LM and choroid plexus vessels, the two vascular junctions between the periphery and CNS. Notably, we did not detect BCCs inside the choroid plexus at any post-engraftment time-points (FIG. 1g,i; FIG. 4g). To track fluorescent tumor cells in the LM vasculature in real-time, we used intravital microscopy (IVM) through thinned skull windows (FIG. 3b,c). Within hours post-intracardiac engraftment, we detected extravasated BCCs in the calvarial BM but not in the LM (FIG. 4b-e). Occasional BCCs were observed in circulation inside LM blood vessels, but did not demonstrate adhesive interactions with the LM vascular lumen nor extravasation into the LM (FIG. 1h,i and FIG. 4d,g). Instead, scattered BCCs were first detected in the LM 3 days post-engraftment, where they appeared on the abluminal surface of the LM vasculature (FIG. 1h,i). In contrast, BCCs lodged within the lumen of brain parenchymal vessels immediately after intracardiac engraftment and extravasated across the blood-brain barrier to infiltrate the surrounding parenchyma, similar to observations in other CNS tropic BC models^{16,17} (FIG. 4f,g). Parenchymal metastases were consistently detected >190 μm from the pia mater making it unlikely that these were seeding the LM (FIG. 4h). These findings suggest that blood-borne 1833-P and EO-LM2 BCCs, while capable of hematogenous metastasis to the brain parenchyma, do not commonly extravasate across the choroid plexus or LM vasculature to invade the LM.

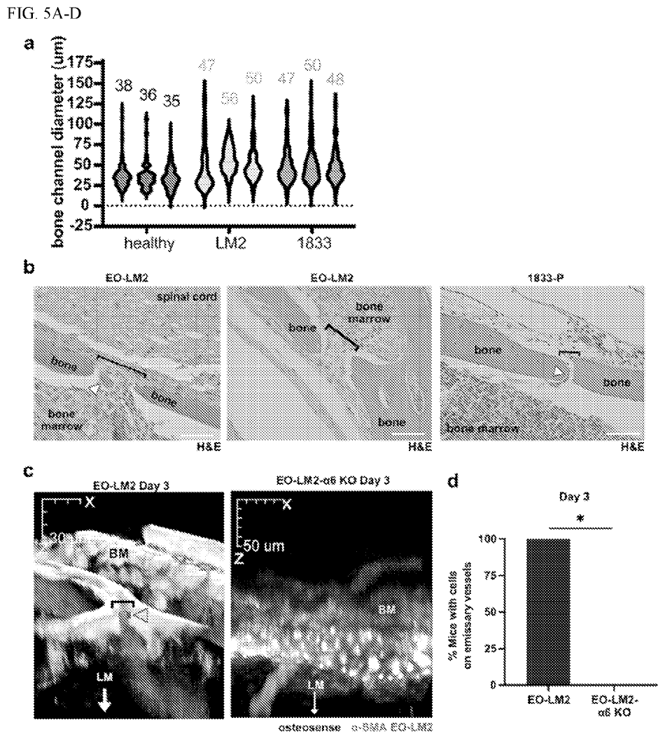

Previous findings from a large autopsy series indicate that the majority of BC patients with LM metastasis have coexisting vertebral or calvarial bone metastases located near prominent sites of LMD involvement⁹. This observation suggests the possibility of direct BM-to-LM metastasis, though a mechanism has never been identified in solid tumors. To determine whether BCCs metastasized to the LM by migrating along the abluminal surface of BM-LM bridging emissary vessels, we performed detailed radiographic and histologic analyses of lumbar vertebrae and ex vivo immunofluorescence (IF) imaging of calvaria^{18,19} in 1833 and EO-LM2-engrafted mice. High resolution microCT imaging demonstrated that mice with LMD had a widening in the mean diameter of vertebral body emissary vessel bone channels, the apertures through which the BM emissary vessels enter into the spinal canal and LM, (FIG. 1j; FIG. 5a), suggesting involvement by disease. We confirmed these radiographic findings by analysis of histologic cross-sections of the spines (FIG. 1k,l; FIG. 4b). At vertebral levels corresponding to BM metastatic involvement in tumored mice, we identified bone channels containing perivascular BCC clusters in transit between BM and adjacent LM (FIG. 1k,l; Extended Data FIG. 5b). Finally, ex vivo imaging of optically cleared calvaria revealed BCCs engaging α-SMA+ vessels¹⁰ within intact emissary bone channels (FIG. 1m; FIG. 5c).

Taken together, these findings reveal a novel route of solid tumor cell migration into the LM.

### Example II

This example demonstrates that integrin α6 expression by BCCs mediates abluminal vascular LM invasion.

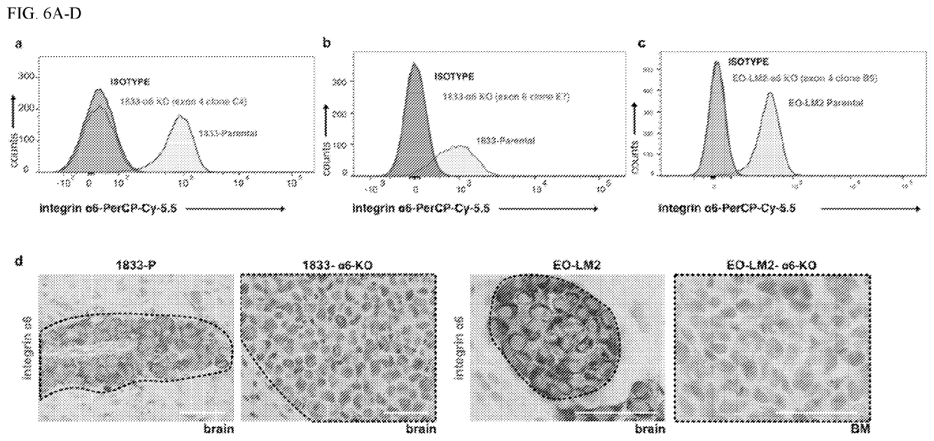

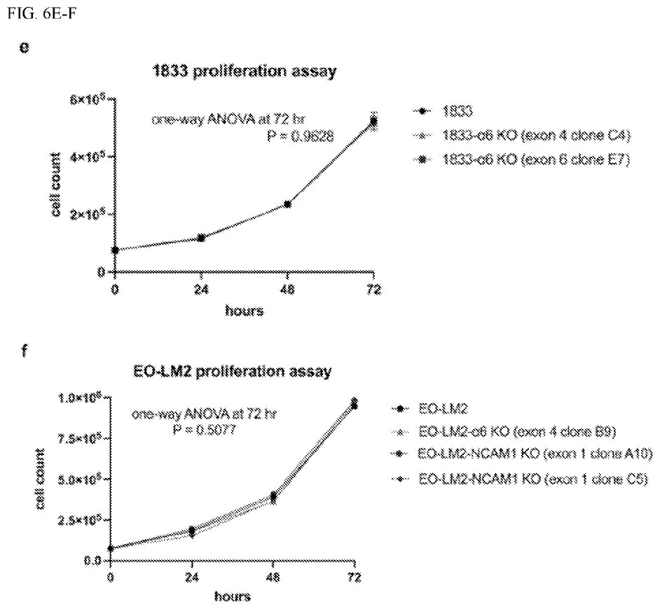

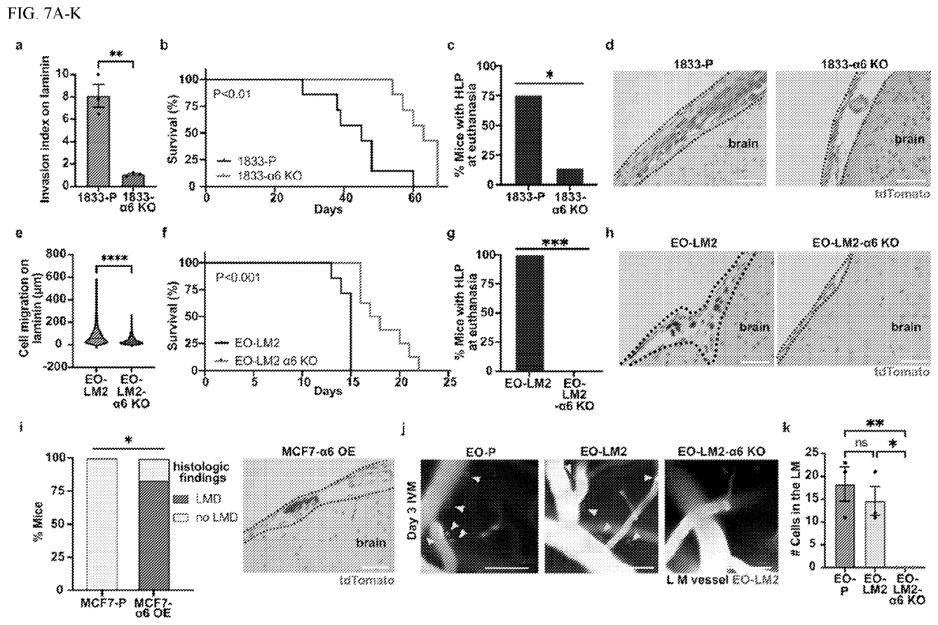

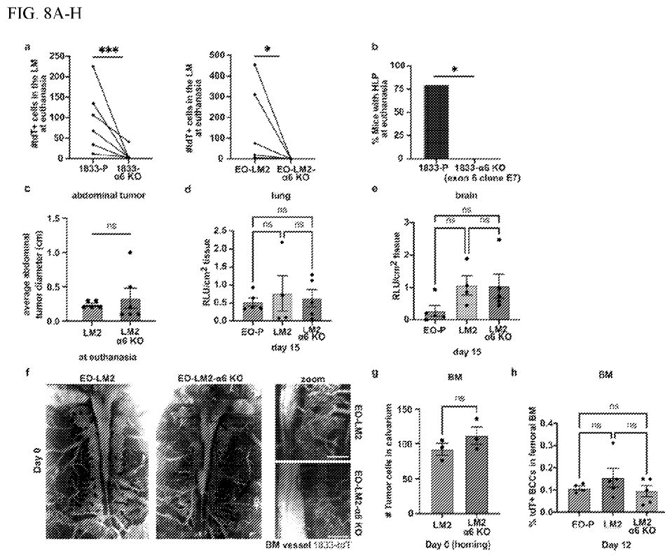

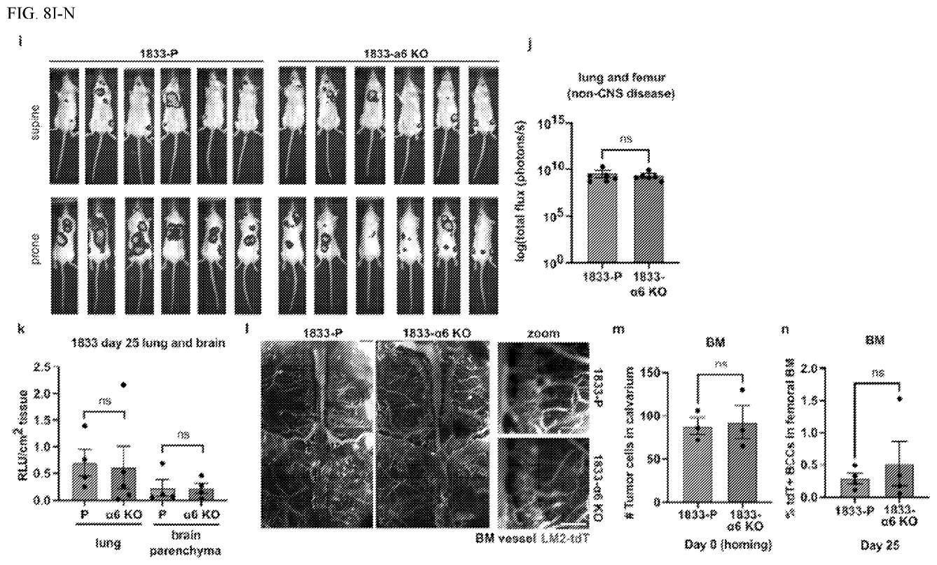

In mouse models of B-ALL, the laminin receptor integrin α6 plays a key role in abluminal LM metastasis along the laminin-rich basement membrane of emissary vessels¹⁰. We therefore investigated whether laminin receptors also regulate BC LM metastasis. We first confirmed the cell surface expression of the high affinity laminin receptor, integrin α6^{20,21}, on both the 1833-P and EO-LM2 cell lines (FIG. 6a-d). We next utilized CRISPR Cas-9 to delete (KO) integrin α6 from these cells. While integrin α6 KO had no effect on in vitro proliferation (FIG. 6e,f), it resulted in a substantial decrease in BCC invasion and migration on laminin (FIG. 7a,e). To assess the contribution of integrin α6 to LM invasion in vivo, we engrafted mice intracardially with either the parental or integrin α6 KO 1833 and EO-LM2 cell lines. In both models, we found that integrin α6 KO resulted in prolonged survival and a decreased incidence of HLP, with histologic analysis confirming a marked decrease in LMD (FIG. 7b-d,f-h; FIG. 8a,b). Integrin α6 KO and parental cells showed equivalent growth in the brain parenchyma, lung, and BM in both models, suggesting the survival and CNS phenotypes were attributable to decreased LM metastasis (FIG. 8c-n).

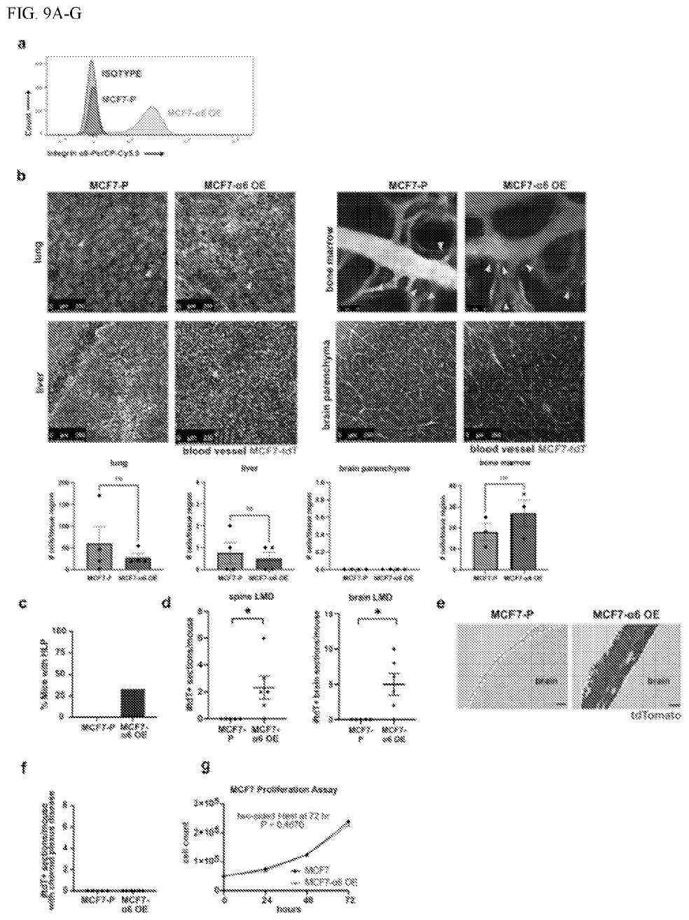

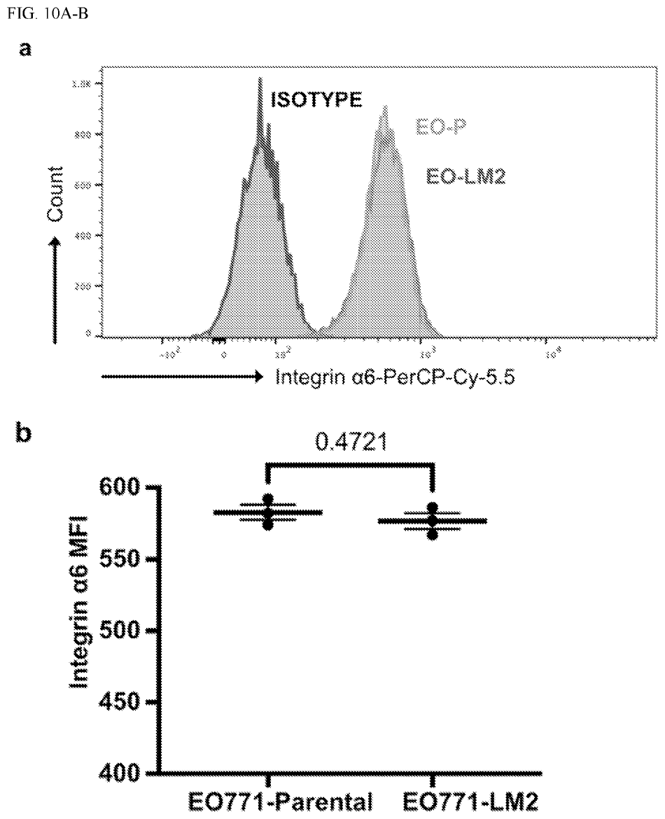

To determine whether integrin α6 is sufficient for LMD development, we overexpressed (OE) integrin α6 in the α6-negative MCF7 cell line (FIG. 9a). MCF7 cells home to the BM and establish micrometastases, but do not metastasize to the LM following intracardiac engraftment (FIG. 7i; FIG. 9b-e). We found that mice engrafted with the MCF7-α6 OE cell line had a significant increase in LMD burden, with 33% of mice engrafted with the MCF7-α6 OE cell line developing HLP (FIG. 7i; FIG. 9c-e). No mice had disease in the choroid plexus (FIG. 9f). Integrin α6 OE did not effect on in vitro cell proliferation or in vivo colonization of the lung, liver or BM (FIG. 9b,g). These data in xenograft and syngeneic mouse models support a critical role for integrin α6 in LMD progression.

The marked decrease in BC LMD progression caused by integrin α6 deletion could reflect a diminished ability of integrin α6 KO cells to penetrate the LM and/or a decreased capacity to proliferate within the LM niche.

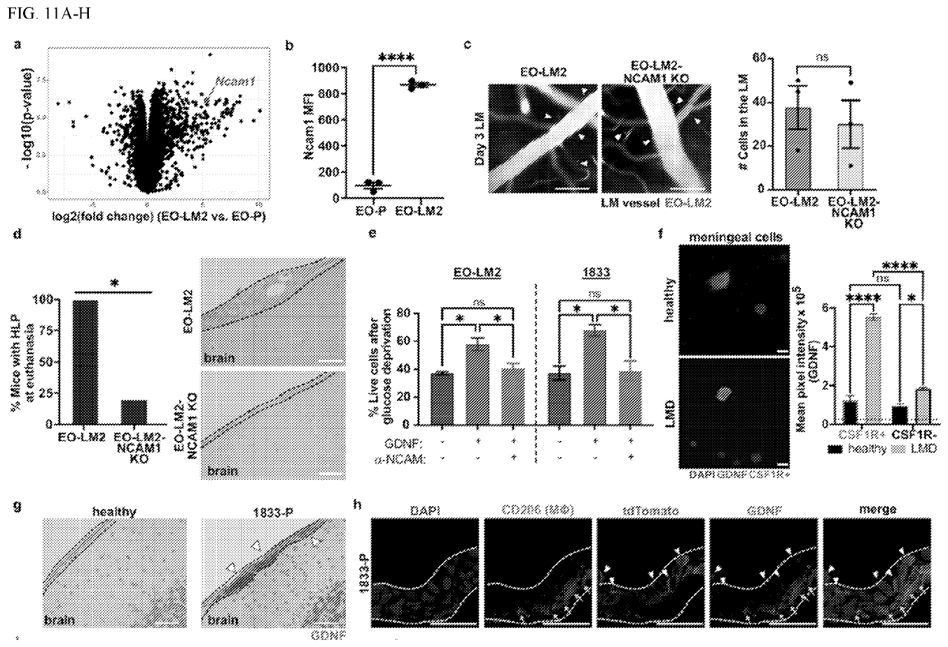

The parental E0771 cell line (EO-P) expresses functional integrin α6 receptors (FIG. 10), yet in contrast to its LM-trophic EO-LM2 subclone, rarely demonstrates LMD progression in vivo (FIG. 1e). We hypothesized that EO-P cells could invade the LM on the basis of their integrin α6 expression, but did not produce overt LMD due to an inability to proliferate in this microenvironment. To test this in vivo, we compared early LM colonization in mice intracardially engrafted with EO-P, EO-LM2 and EO-LM2-α6 KO cells using highly sensitive intravital microscopy. While EO-LM2-α6 KO cells were rarely detected in the LM or in transit along emissary vessels, EO-P and EO-LM2 cells were present in the LM at similar levels early post-engraftment (FIG. 7j,k). These data suggest that integrin α6 functions to mediate LM colonization, but another factor(s) differentially expressed by EO-LM2 cells mediates their successful growth in the LM niche.

### Example III

This example demonstrates GDNF/NCAM signaling in the LM supports BCC survival in a subverted perivascular macrophage niche.

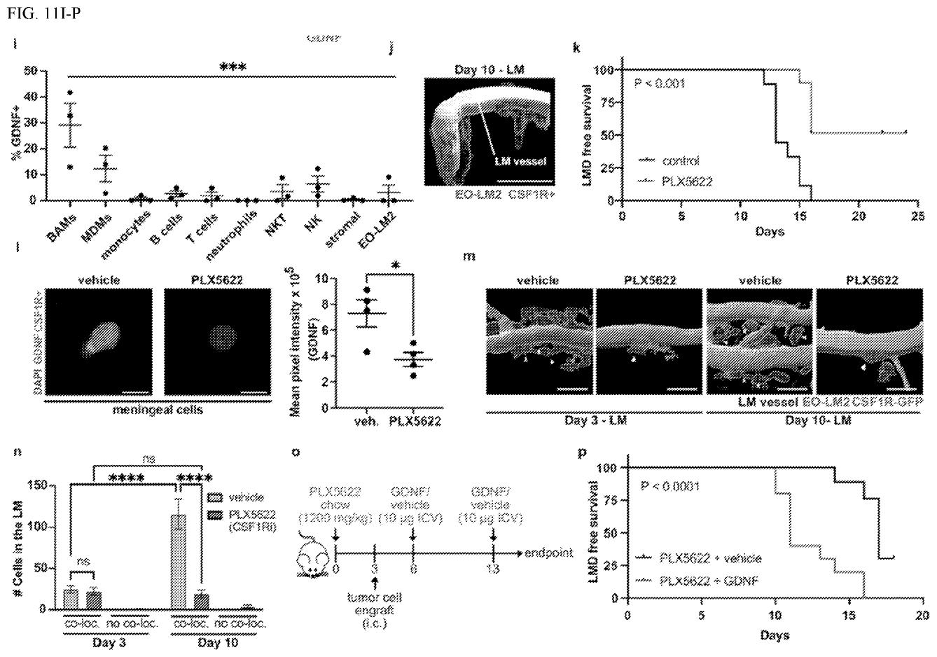

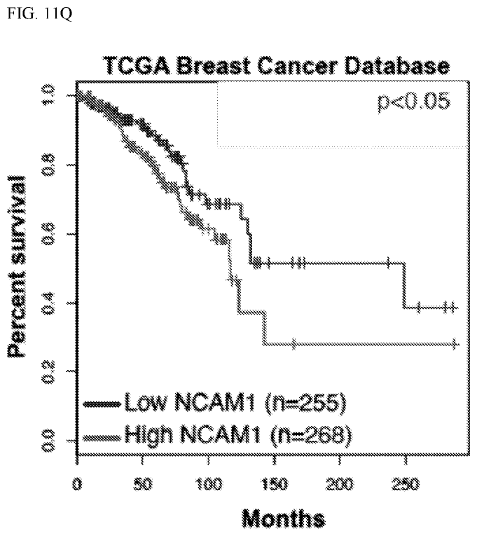

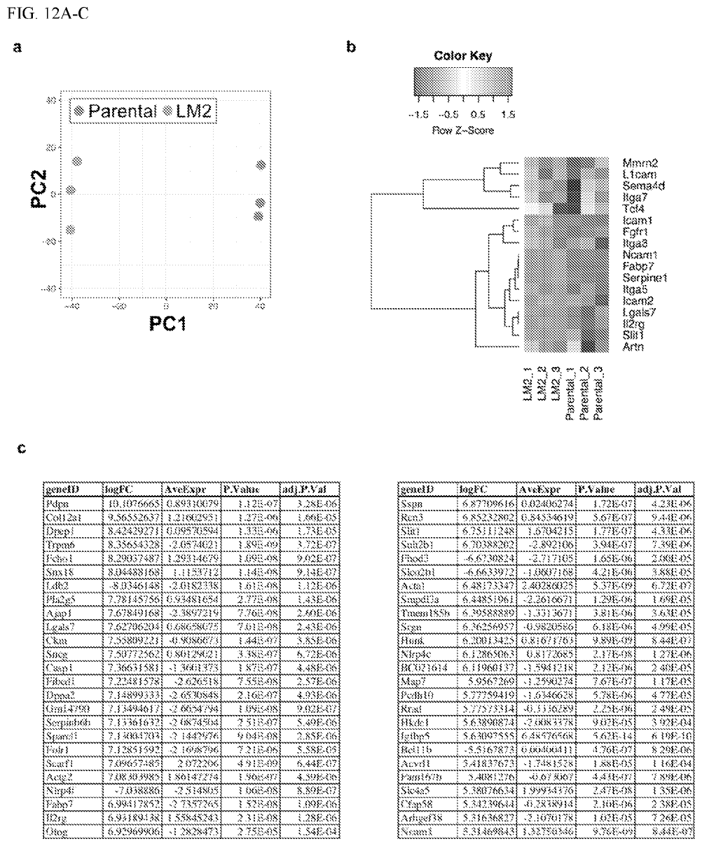

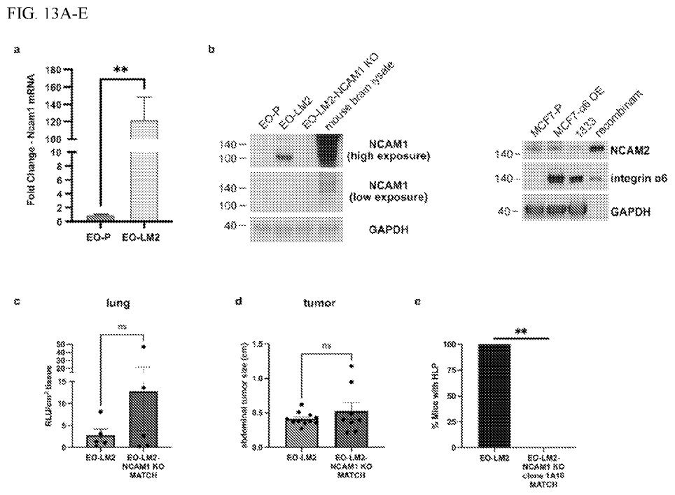

To screen for signaling pathways in EO-LM2 cells that could support their survival or proliferation in the meningeal niche, we performed RNA-seq analysis of EO-P vs. EO-LM2 cells. We found that Ncam1 was one of the most highly upregulated transcripts in EO-LM2 vs. EO-P cells (FIG. 11a; FIG. 12). We confirmed the cell surface expression of NCAM1 on EO-LM2 cells and absence in EO-P cells (FIG. 11b; FIG. 13a,b). NCAM1 deletion from EO-LM2 cells did not alter in vitro proliferation, systemic disease growth or initial colonization of the LM (FIG. 11c; FIGS. 6f and 13c,d). However, NCAM1 KO inhibited LMD progression (FIG. 11d; Extended Data FIG. 13e), indicating its role in BCC survival rather than invasion of the LM niche.

NCAM1 and its paralog NCAM2 (collectively referred to as NCAM) encode homophilic cell-cell adhesion molecules that have also been shown to form part of a cell surface signaling complex for the neurotrophin, GDNF^{22,23}. While GDNF is not highly expressed in the healthy adult LM, it plays a crucial role in nervous system development and has been shown to be secreted by CNS microglia and macrophages in response to brain injury, where it blocks apoptotic neuronal stress responses²⁴⁻²⁹. The LM is a relatively nutrient-poor environment, presenting tumor cells with additional cellular stress³⁰. We therefore tested whether GDNF could protect NCAM+1833 (FIG. 13b) and EO-LM2 cells from cell death induced by glucose deprivation. Recombinant GDNF treatment provided marked protection from cell death in low glucose conditions, and this effect was reversed by NCAM receptor blockade (FIG. 11e).

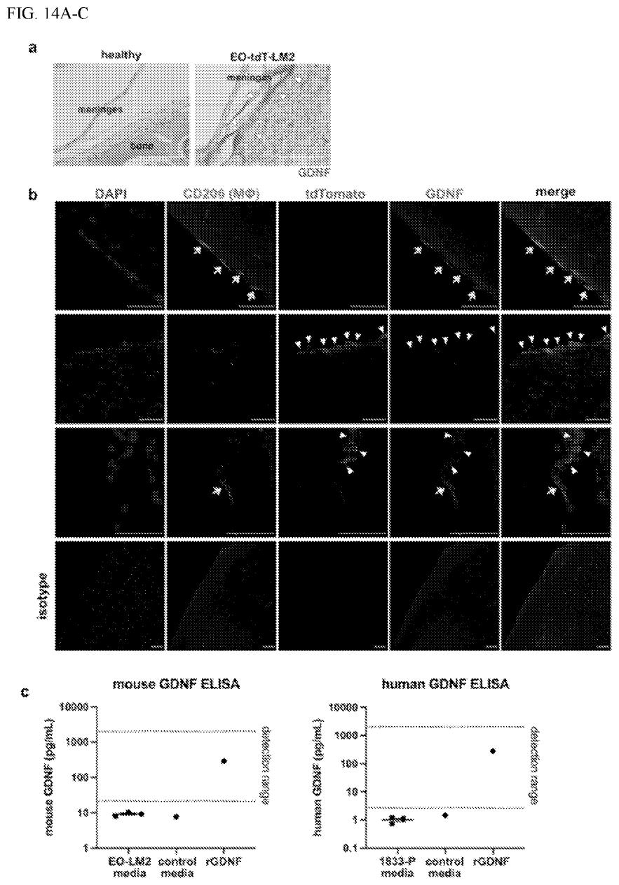

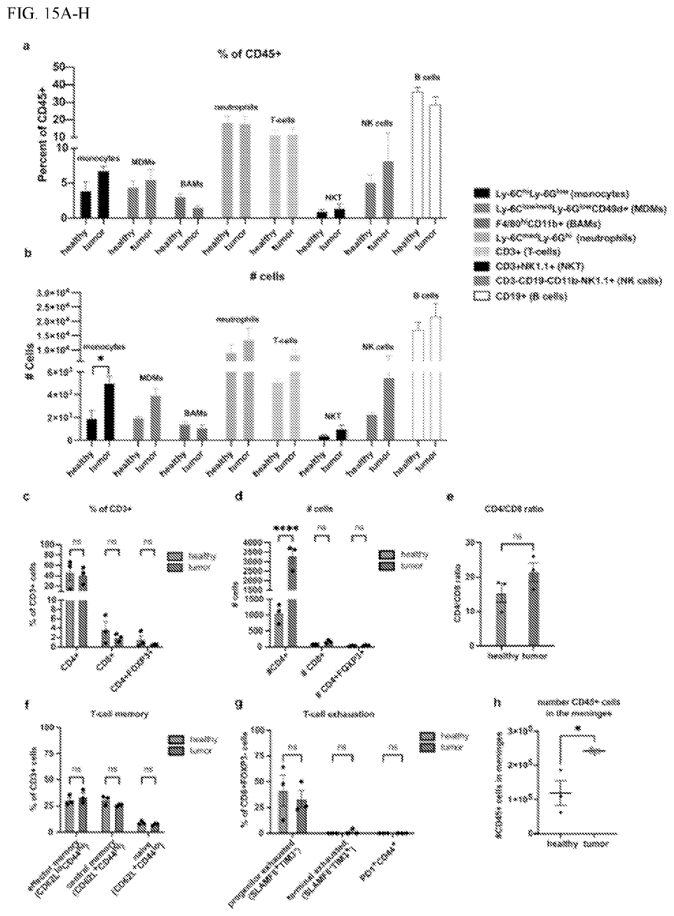

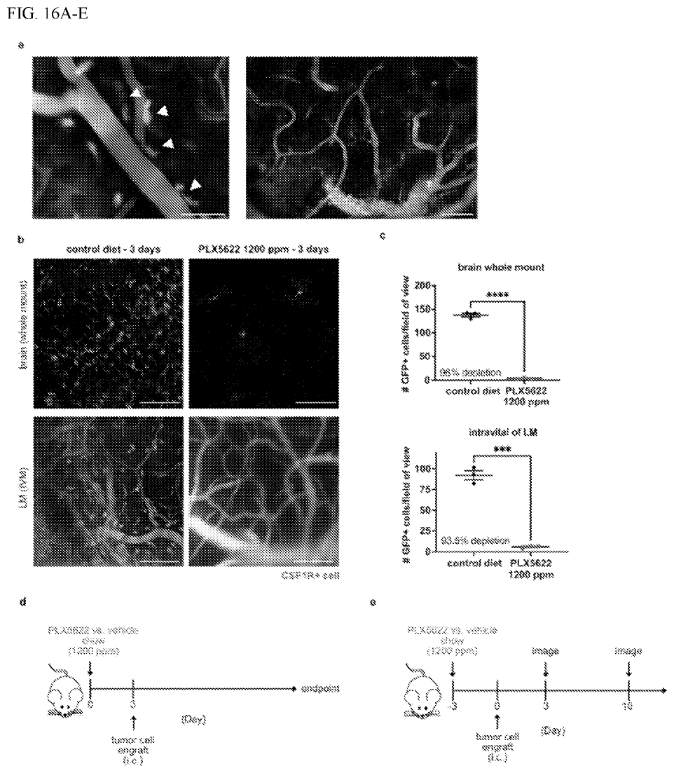

To determine if BCCs stimulate GDNF expression in meningeal macrophages which are CSF1R+, we isolated meningeal cells from healthy vs. EO-LM2-engrafted CSF1R-GFP C57BL/6 reporter mice and performed GDNF IF. We found that CSF1R+ cells exhibited over a 3-fold increase in GDNF expression, while CSF1R− meningeal cells displayed only a modest increase in GDNF expression (FIG. 11f). GDNF IHC confirmed a marked increase in GDNF deposition within the LM of diseased mice (FIG. 11g; Extended Data FIG. 11a). Co-immunofluorescence staining revealed that CD206+ macrophages, but not tdT+ tumor cells, were a source of GDNF (FIG. 3h; FIG. 14b). To better define the GDNF+ cell populations within the meninges, we conducted flow cytometry on meningeal isolates from EO-LM2 endpoint mice (Supplemental Information 1). This revealed that border associated macrophages (BAMs) and monocyte-derived macrophages (MDMs) were the predominant source of GDNF, while other meningeal cell populations, including monocytes, neutrophils, lymphocytes, NK cells, stromal cells, and tumor cells, showed no or minimal GDNF expression (FIG. 11i; FIG. 14c). Quantitative analysis revealed stable numbers of BAMs in tumored mice in contrast to a marked, although non-significant, increase in MDMs, suggesting the potential recruitment of MDMs to the diseased LM (FIG. 15).

We next performed intravital imaging of the LM of tumored CSF1R-GFP mice to examine whether there was evidence of in vivo interactions of LM macrophages and metastatic BCCs. We observed a near 100% co-localization of EO-LM2 and CSF1R+ myeloid cells (FIG. 16a). 3D reconstruction of Z-stack images revealed BCCs tightly insinuated between blood vessels and perivascular macrophages (FIG. 11j), suggesting their protective encasement by CSF1R+ cells.

To determine whether CSF1R+ cells in the LM niche were indeed pro-tumoral, we treated mice with the CSF1R inhibitor, PLX5622, which dramatically depletes CSF1R+ cells from the CNS with modest effects on systemic CSF1R+ populations (FIG. 16b-d)^{31,32}. We found that CSFTR+ cell depletion substantially prolonged time to LMD progression in EO-LM2 mice, consistent with a dramatic decrease in LM cells expressing GDNF (FIG. 11k,l).

Notably, CSF1R+ cell depletion by PLX5622 did not affect initial BCC invasion of the LM, but did prevent subsequent BCC expansion within the LM (FIG. 11m,n; FIG. 16e), indicating that resident macrophages promote LMD by supporting BCC survival and proliferation within the LM niche. Lastly, we tested whether intraventricular delivery of recombinant GDNF could rescue LM tumor growth in meningeal macrophage-depleted mice. As shown in FIG. 11o,p, intraventricular administration of GDNF accelerated LMD progression in tumored, CSF1R-depleted mice, suggesting that GDNF was sufficient to replace the pro-tumoral effects of macrophages. Finally, data mining of The Cancer Genome Atlas revealed that breast cancer patients with high NCAM expression have worse overall survival compared to patients with low NCAM expression (FIG. 11q).

### Example IV

This example demonstrates that integrin α6 and GDNF expression are associated with meningeal metastasis in BC patients.

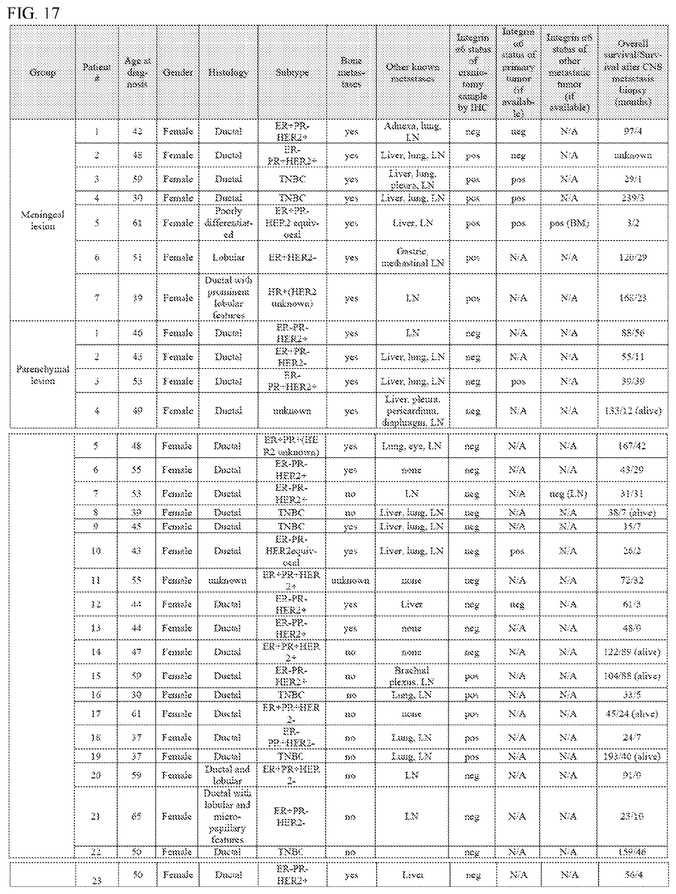

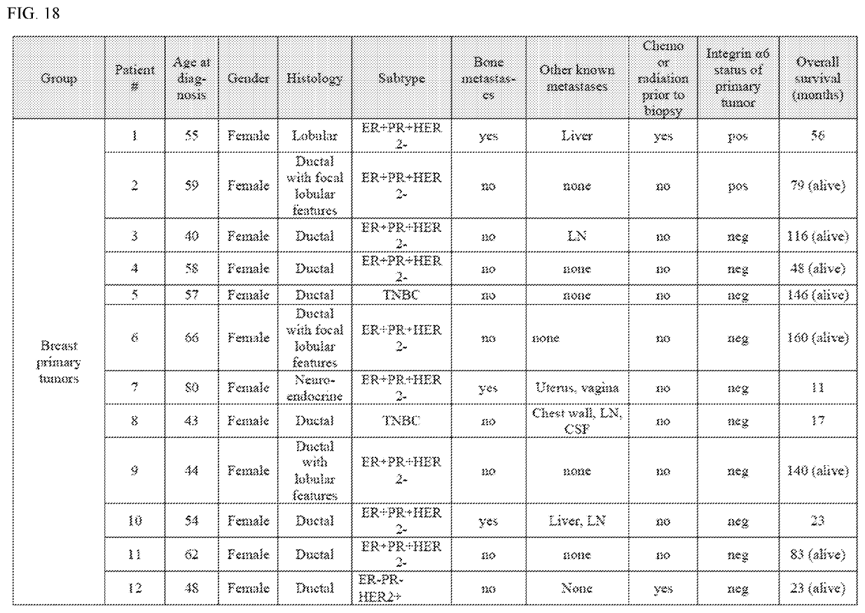

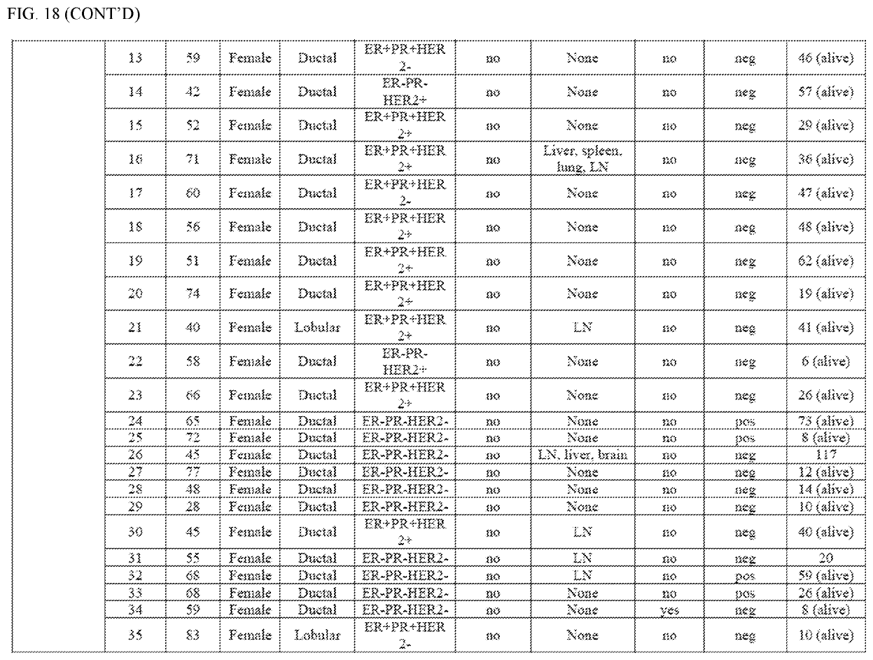

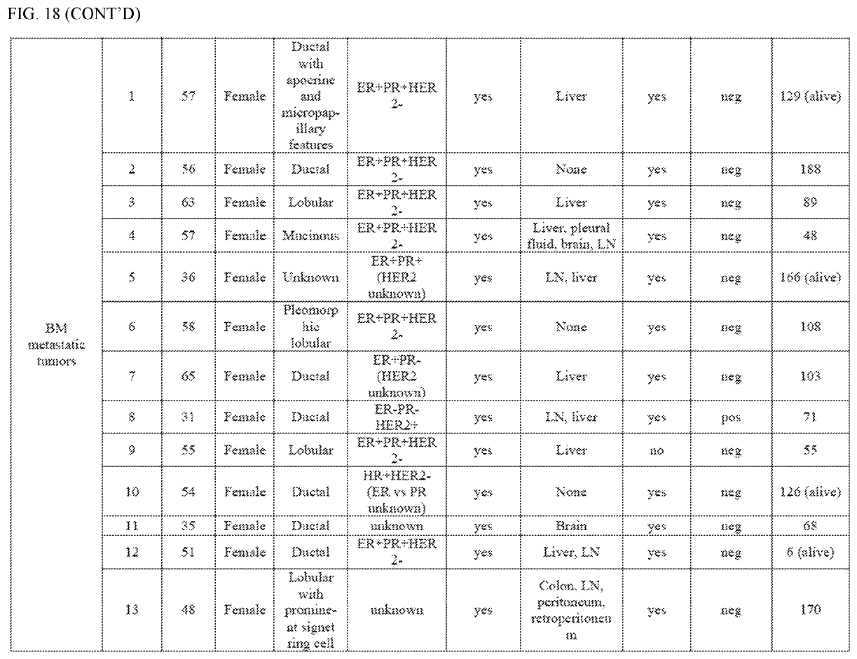

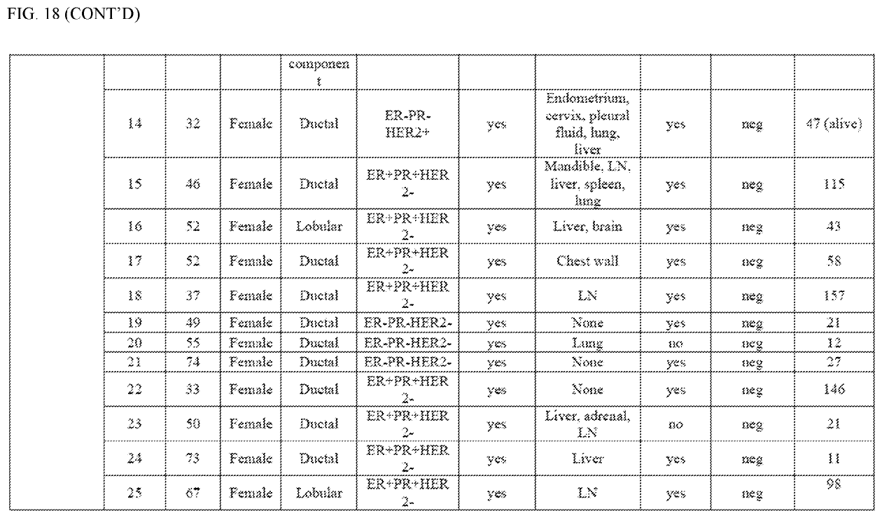

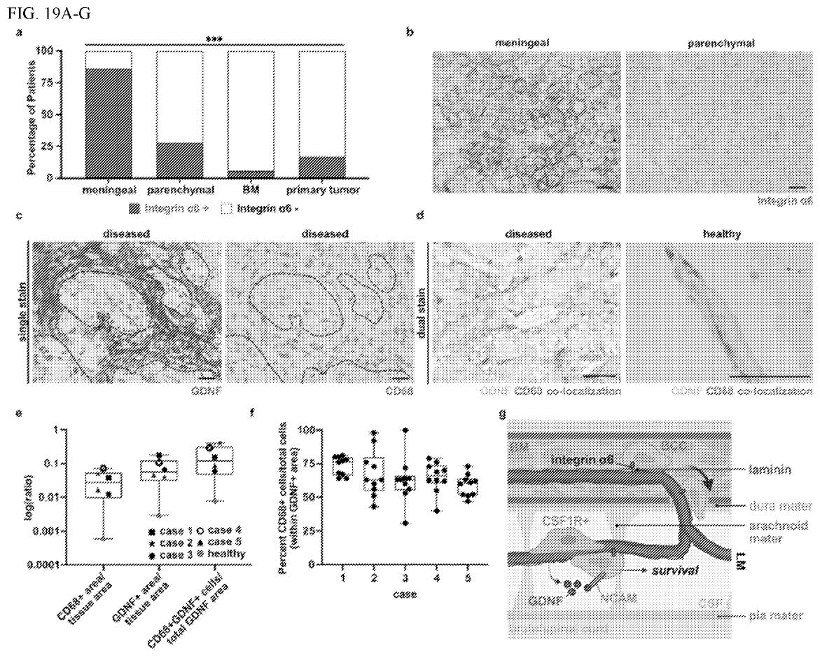

We next examined whether integrin α6 receptor and/or GDNF expression correlated with meningeal metastasis in patients with BC diagnoses. We used IHC to compare tumor cell membrane integrin α6 expression in breast primary tumors, BM metastases, and brain biopsy specimens from patients with meningeal-based vs. brain parenchymal BC metastases (FIGS. 17 and 18). CT and MRI imaging were reviewed to exclude any patients with breakdown of the calvarial or vertebral bone that would allow unconstrained spread to the LM. Although the rarity of biopsies performed on meningeal-based lesions limited our sample size, we found a statistically significant correlation between integrin α6 positivity and meningeal lesions (FIG. 19a,b). Review of the medical record confirmed that all patients with meningeal lesions also had bone metastasis detected by bone scan or by BM biopsy.

Finally, we performed IHC staining for GDNF and CD68 expression in available cases from our panel of meningeal-based metastasis biopsy specimens (FIG. 19c,d). We found that 5/6 of these samples showed GDNF positivity characterized by intense staining of the peri-tumoral stroma. Machine learning (FIG. 19e) and pathologist's quantification (FIG. 19f) of GDNF and CD68 staining on these 5 positive cases revealed that CD68+ cells frequently co-localized with GDNF, and that the majority of cells within GDNF+ regions were CD68+. These data suggest macrophages as an important source of GDNF deposition in patients.

The LM are considered a “sanctuary site” for tumor growth, due to the limited access of both cytotoxic immune cells and therapeutic agents in this anatomic compartment³³. At the same time, it is a relatively harsh environment for tumor cell survival, requiring adaptation to its hypoxic and nutrient-poor conditions³⁰. The molecular processes that allow BC and other malignant cells to navigate and flourish in this microenvironment are only beginning to be elucidated³⁴⁻³⁶. Here we show that BCCs exploit a hematopoietic cell trafficking corridor¹⁰ to enter the LM. There, they subvert resident macrophages to create a favorable microenvironment for growth. This process combines neuronal mimicry, immune cell hijacking, and a restructuring of the LM to induce a more protective stromal niche. Central to this process are integrin α6 and NCAM, each of which has well-described roles in neurogenesis and neuronal pathfinding³⁷⁻⁴⁰. These molecules appear to play tandem roles in BC LMD, with BC integrin α6-vascular basement membrane laminin interactions stimulating migration along the vasculature⁴¹ of BM-LM bridging vessels, and NCAM transducing growth and survival signals from meningeal GDNF. This is highlighted by the observation that mice engrafted with the EO-P (integrin α6+, NCAM−) and MCF7-P (integrin α6−, NCAM+) (FIG. 13b) cells do not develop LMD; however, upon acquisition of both molecules, the EO-LM2 (integrin α6+, NCAM+) and the MCF7-α6 OE (integrin α6+, NCAM+) cells are able to successfully invade and colonize the LM. Interestingly, NCAM and other integrins have been reported to co-localize at the cell surface, and functional interactions between integrins and NCAM can reciprocally enhance downstream signaling events^{42,43}. Integrin α6 expression may therefore not only propel LM invasion but also strengthen the metastatic “fitness” of cells in the laminin-rich meningeal microenvironment.

Macrophages are increasingly recognized to play a myriad of roles in primary brain tumors and metastases⁴⁴⁻⁴⁸, although their function in the unique microenvironment of the LM is only beginning to be understood^{34,49,50}. Residing in close apposition to the vasculature, meningeal macrophages are ideally situated to perform a gatekeeper function. Despite this, our findings suggest a model in which BCCs in the LM are able to subvert these macrophages, paradoxically by stimulating neuroprotective macrophage inflammatory responses. Arrival of BCCs into the LM by means of a perivascular migration route may also ensure that BCCs enjoy the protection of perivascular macrophages throughout their journey. Indeed, we observed invading LM BCCs to be tightly intercalated between the vasculature and perivascular macrophages. This close interaction, perhaps enhanced by homotypic NCAM cell adhesion between BCCs and NCAM+ macrophages, may provide a combined physical and molecular shield. Going forward, it will be important to fully characterize the unique phenotype of meningeal macrophages and other potential interactions between BCCs and meningeal macrophages that promote LM tumor growth, including conceivable effects on adaptive immune responses.

It will also be important to understand other potential sources of GDNF, additional molecular mechanisms that may regulate abluminal vascular migration between the BM and LM, and whether BCCs may employ hematogenous and abluminal migration simultaneously. As other LM metastatic malignancies, including lung cancer and melanoma, can also express integrin α6 and/or NCAM, it is possible that these pathways are tumor agnostic. Future efforts to targets these signaling mechanisms could therefore benefit BC and other LM metastasis patients who have a high unmet clinical need for effective therapies.

### Example V

This example demonstrates that integrin α6 plays a role in BC survival in the leptomeningeal (LM) niche.

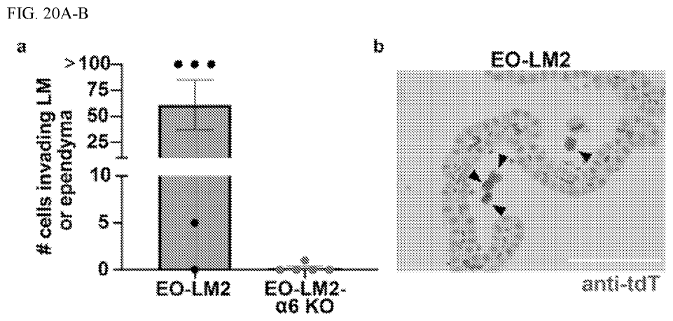

Integrins can transduce multiple downstream signals regulating cell proliferation and resistance to apoptosis. Our previous data demonstrate that integrin α6 plays a critical role in tumor invasion and colonization of the LM. In order to study whether integrin α6 could play a role in LMD growth after tumor cell migration to the LM, we engrafted EOLM2 or EO-LM2-α6 knockout (KO) breast cancer cells directly into the LM by intracerebroventricular (icv) injection. Analysis of brain histologic sections 2 weeks post-icv engraftment showed LMD in 4/5 EO-LM2 mice, but virtually no detectable disease in EO-LM2-α6 KO mice, supporting dual roles for α6 in breast cancer LM colonization and proliferation (see FIG. 20).

### Example VI

This example provides the material and methods implemented in conducting the experiments described in Examples I, II, III, IV, and V.

### Cell Lines and Culture

1833 cells were obtained from the Massague Lab (Memorial Sloan Kettering Cancer Center, New York, NY) and cultured in high-glucose Dulbecco's modified Eagle's medium (DMEM; Corning) supplemented with 10% fetal bovine serum (FBS, Gemini Bio-Products). The E0771 (CH3 BioSystems) cell lines were cultured in RPMI 1640 medium (Corning) supplemented with 10% FBS and 10-mM HEPES (Thermo Fisher). MCF7 cells (ATCC) were cultured in high-glucose Dulbecco's modified Eagle's medium (DMEM; Corning) supplemented with 10% fetal bovine serum (FBS, Gemini Bio-Products). All cultures were maintained at 37° C. in a 5% CO₂ humidified atmosphere. STR profiling was repeated routinely to authenticate cell lines. Mycoplasma testing by PCR was performed every three months on all cell lines in culture.

Generation of 1833-tdTomato-Luciferase and E0771-tdTomato Cell Line

The tdTomato-luciferase vector was constructed by cloning the tdTomato gene from the pLX-IRES-tdTomato vector (Clontech) into the pGL3 luciferase promoter vector (Promega). The 1833-tdTomato-luciferase (1833-P) and MCF7-tdTomato-luciferase (MCF7-tdT) cells were generated through the lentiviral transduction of the tdTomato-luciferse vector into 1833 cells and then sorting single cell clones. The E0771-tdT cells were generated though the lentiviral transduction of the FUtdTW plasmid (Addgene) into E0771 cells and then sorting single cell clones.

### Derivation of the E0771-LM2 Cell Line

6- to 8-week-old female C57BL/6 (Jackson Laboratories) mice were engrafted with 1 ×10⁵ E0771-tdT cells in PBS via intracardiac injection. One mouse that developed hind limb paralysis was euthanized and the spinal cord and meninges of each vertebral body were then washed with RPMI 1640 containing 10% FBS to collect cells from the CSF. The CSF cells were passed through a 70-μm filter, washed with PBS, and treated with ACK lysis buffer to remove red blood cells. The cells were then plated in a 12.5-cm tissue culture flask and cultured for 2 weeks in culture medium containing Penicillin-Streptomycin (Thermo Fisher) and Antibiotic-Antimycotic (Thermo Fisher). The medium was changed daily. Once the isolated E0771-LM1-tdT cells were confluent, the E0771-LM2-tdT cells were generated by repeating the process above.

### CRISPR-Cas9 Knockout of Integrin α6 and Ncam1

The CRISPR-Cas9 mediated knockout cell lines were created by transfecting the cell lines with the pSpCas9(BB)-2A-GFP (PX458) plasmid (Addgene) (Ran, F. A. et al. Genome engineering using the CRISPR-Cas9 system. Nature Protocols 8, 2281-2308 (2013)). Benchling was used to design the target sequences of the sgRNAs which were cloned into the PX458 plasmid. Target sequences: human ITGA6 sgRNA1, TCTCAGGATTGAAGACGATA (SEQ ID NO: 2); human ITGA6 sgRNA2, GAGCACATATTCGATGGAGA (SEQ ID NO: 3); mouse Itga6, GCACAGTGAGCGTGAGCCGGC (SEQ ID NO: 4), mouse Ncam1, GGCGGCGGAGCAATGGACCG (SEQ ID NO: 5). EO-LM2 and 1833 cells were grown to 70% confluency in a 6-well plate and transfected with 2.5 μg of plasmid DNA in Opti-MEM (Thermo Fisher) with Lipofectamine 3000 (Thermo Fisher). After 24 h the media was changed and after 48 h the transfected cells were stained with Live/Dead Fixable Near IR (Thermo Fisher) and live single cells were sorted for the GFP reporter into a 96-well plate. Single cell clones were grown up and were assessed for KO via flow cytometry and Western blot.

### Overexpression of Integrin α6

MCF7-tdT cells were transduced with lentivirus containing the vector pLX_TRC317-ITGA6 (clone TRCN0000479977) (Millipore Sigma) and selected using puromycin. Overexpression was confirmed via flow cytometry and Western blot.

### Mouse Engraftment

Specific pathogen-free 6- to 8-week-old female SCID mice (Charles River) were engrafted with 1×10⁵ 1833-P cells in 100 μL of PBS via intracardiac injection. 6- to 8-week-old female C57BL/6 (Jackson Laboratories) and MacGreen (Jackson Laboratories) mice were engrafted with 1×10⁵ EO-LM2 cells in 100 μL of PBS via intracardiac injection. Specific pathogen-free 6- to 8-week-old female NCG mice (Charles River) were engrafted with 1×10⁵ MCF7 cells in 100 μL of PBS via intracardiac injection. All experimental procedures involving mice were approved by the Animal Care and Use Committee of Duke University.

### Administration of PLX5622

PLX5622 (ChemGood) was formulated into AIN-76A rodent chow at 1200 mg kg⁻¹ by Research Diets Inc. Formulated chow was irradiated prior to administration. CSF1R-GFP (MacGreen) mice were given AIN-76A±PLX5622 for three days prior to engraftment and continued to feed on formulated or control chow for the duration of the study.

### Skull Thinning

Mice were anaesthetized using isoflurane and the cortical bone was exposed following a sagittal incision in the scalp. The head of the mouse was secured using a stereotactic apparatus. A micromotor high-speed drill (Stoelting) with a carbide 0.75-mm drill bit (Stoelting) was used to thin the skull immediately to the left of the sagittal suture and centered between the coronal suture and the lambdoid suture. The skull-thinned region was circular and approximately 1 mm in diameter and the thinning continued until the meningeal vessels were visible under a dissecting microscope and the remaining bone was about 50 μm thin. PBS was used to clean the surgical site and dissipate heat every 15 sec. High-resolution images of the leptomeninges were obtained through the skull thinned window using a Leica SP8 confocal and multiphoton microscope with a 25× water immersion lens.

### Intravital Imaging and Whole-Mount Imaging

Mice were anaesthetized using isoflurane and a rectangular incision was made in the scalp, revealing the intact, underlying cortical bone allowing for the skull thinning procedure. The skull-thinned region was washed with PBS and fluorescently labelled dextran (Dex-AF647) was administered via tail vein injection to highlight the vasculature. Dex-AF647 is a large molecular weight dextran molecule conjugated to the fluorophore Alexa Fluor 647. Because it has a prolonged blood-pool half-life, it is a useful means of visualizing the vasculature in in vivo studies. Mice were placed in a specially designed restrictor and a cover slip was placed over the exposed calvarial bone and surgical window. Mice remained anaesthetized throughout the procedure. High-resolution images were obtained or the BM or through the skull-thinned window using a Leica SP8 confocal and multiphoton microscope with a 25× water immersion lens. The pinhole was set to 2 area units. Visible lasers at 552 nm and 638 nm were used to excite tdTomato and AF647 respectively. A multiphoton laser (Chameleon) tuned to 925 nm was used to excite GFP. HyD detectors were used to detect tdTomato and AF647 and an internal non-descanned detector (NDD) was used to detect GFP. Images and Z-stacks were captured using Leica LASX software using line and frame averaging. A motorized stage was used to scan and take images of the entire skull thinned window (˜1×1 mm) or the entire calvarial bone marrow. After the procedure, the mice were euthanized via carbon dioxide overdose and the brain or lung was immediately removed and placed in a cold PBS bath. A sagittal slice from the midline or the right lung lobe was taken and the tissue was placed on a glass slide and mounted with PBS and a coverslip. Using a 10× objective and motorized stage, the entire slice was imaged using the SP8 system. After scanning, the 25× water objective was used to take higher-resolution images of cells in the brain parenchyma.

### Quantification of Intravital and Whole Mount Imaging Data

Intravital and whole mount images were imported into ImageJ for quantification. Intravital: The number of tdT+ tumor cells were enumerated within the entire imaging window (˜3 mm²) for each mouse. Whole mount: Thresholding was used to remove any background in the tdT channel based off an engrafted mouse control. Tissue bordered were then traced and the mean tdT signal was measured over total tissue area.

### Imaris 3D Reconstruction

Confocal image Z-stacks were imported into Imaris 9.0 (Oxford Instruments), and the contour surface tool was used to create 3D reconstructions.

### Tissue Immunohistochemistry (IHC) and Immunofluorescence (IF)

tdTomato, GDNF, and integrin α6 immunohistochemistry staining were performed under the following conditions. tdTomato: antigen retrieval was performed using EDTA Target Retrieval solution (DAKO). Slides were stained with anti-RFP (Rockland, 5 g mL⁻¹). DAKO Envision System-HRP (DAB), for use with rabbit (DAKO), was used as a secondary antibody. GDNF: antigen retrieval was performed using Citrate Target Retrieval solution (DAKO). Slides were stained with anti-GDNF (Abcam, 2 μg mL⁻¹). DAKO Envision System-HRP (DAB), for use with rabbit (DAKO), was used as a secondary antibody. α6 integrin: Antigen retrieval was performed using EDTA Target Retrieval solution (DAKO). Slides were stained with anti-integrin α6 (Abcam, 6 μg mL⁻¹). DAKO Envision System-HRP (DAB), for use with rabbit (DAKO), was used as a secondary antibody. Human CD68 staining was conducted at the Duke Research Immunohistochemistry Laboratory core. tdTomato, GDNF, CD206 co-immunofluorescence staining were performed under the following conditions: antigen retrieval was performed using Citrate Target Retrieval solution (DAKO). Slides were stained with anti-RFP (Rockland, 5 μg mL⁻¹), anti-GDNF (Abcam, 5 μg mL⁻¹), and anti-CD206 (Biorad, 5 μg mL⁻¹) overnight at 4° C. Secondary antibody staining with donkey anti-rat AF488 pAb (Thermo, 5 μg mL⁻¹), donkey anti-goat AF555 pAb (Thermo, 5 μg mL⁻¹), donkey anti-rabbit AF680 pAb (Thermo, 5 μg mL⁻¹) for 1 hour at room temperature.

### Quantification of IHC

For quantification of disease burden in 1833/EO-LM2 parental vs. α6 KO engrafted mice, 10× brain sections were stained for tdT and the number of tdT+ nucleated cells in the LM were enumerated on each section. Data were not normally distributed and many values in the α6 KO arm were zero, so the difference in cell number between matched mouse pairs was taken and then log-transformed prior to running a one-sample t-test against zero. For quantification of disease burden in the MCF7 model, 14× spine sections and 10× brain sections were stained for tdT. LMD positive cases were defined as mice with at least two distinct sections containing at least 3 tdT+ nucleated cells. The number of sections with tdT+ nucleated cells were also enumerated.

### RNA Sequencing and Analysis

Cells were cultured in tissue culture treated flasks for 48 h to about 75% confluence and RNA was extracted using a Qiagen RNeasy kit (Qiagen). RNA QC, library preparation, and sequencing was conducted by the Duke Center for Genomic and Computational Biology core. RNA concentration was evaluated by Qubit and the integrity was assessed by 2100 Bioanalyzer. Sequencing was conducted using the IlluminaNovaSeq 6000 platform on an S-Prime 50-bp paired-end flow cell. 33 million reads per sample were conducted. Raw reads were mapped to the mouse reference transcriptome, GRCm39 from Ensembl, using Kallisto version 0.46.2. The quality of the reads and the Kallisto mapping was assessed using FastQC and MultiQC. After read mapping, the data were imported into RStudio version 1.4.1106 and edgeR version 3.32.1 was used to calculate normalized log 2 counts per million (CPM) and filtering was carried out to remove lowly expressed genes (genes with less than 1 CPM in at least 3 or more samples). To identify differentially expressed genes (DEGs), precision weights were first applied to each gene based on its mean-variance relationship using VOOM, then data were normalized using the TMM method in edgeR. Linear modeling and bayesian stats were employed via Limma version 3.46.0 to find genes that were up- or down-regulated by 2-fold or more, with a false-discovery rate (FDR) of 0.05. All graphics and data wrangling were handled using the tidyverse suite of packages (version 1.3.1). The workflow used was initially described by Berry, et al. (Berry Alexander, S. F. et al. An Open-Source Toolkit To Expand Bioinformatics Training in Infectious Diseases. mBio 12, e01214-01221 (2021)).

### High-Resolution X-Ray Computed Tomography

Mouse spines were dissected and fixed for 24 h in cold 10% formalin. Fixed spines were dehydrated in a chemical cabinet for 72 h at room temperature and then the dehydrated spines were wrapped in Kimwipes and placed snuggly into a plastic straw for imaging. Four lumbosacral vertebrae per specimen were imaged at the Duke Shared Materials Instrumentation Facility using a high-resolution microCT (Nikon XTH 225 ST) with a 180 kV NanoTech Transmission Based Signal Tungsten Target at a 6-μm³ isotropic voxel size and 4000-ms exposure. The scans were reconstructed using Nikon's Feldkamp Cone Based CT Algorithm and then imported into Avizo 9.5 for analysis. The isosurface rendering tool was used to create 3D reconstructions and measure bone channel diameters by a blinded experimenter.

### Isolation of Mouse Meningeal Cells

The mouse meninges were isolated as described by Louveau et al (Louveau, A., Filiano, A. J. & Kipnis, J. Meningeal whole mount preparation and characterization of neural cells by flow cytometry. Current protocols in immunology 121, e50-e50 (2018)). Briefly, the skull cap was removed and placed in a cold PBS bath under a dissecting microscope. Using fine forceps, the meninges were carefully peeled off the skull. Any remaining meninges on the brain surface were carefully removed with fine forceps while limiting the removal of residual brain parenchyma. The meninges were digested for 45 min at 37° C. in digest buffer (1×HBSS with calcium and magnesium (Corning), 5% FBS, 10 mM HEPES, DNase (Sigma Aldrich, 20 U mL⁻¹), Liberase DL (Sigma Aldrich, 10 mg mL⁻¹), Liberase TL (Sigma Aldrich, 10 mg mL⁻¹)) and lightly shaken every 10 min. The digested solution was then passed through a 70-μm cell strainer and a rubber syringe pump was used to gently push through any undigested tissue. Red blood cells were lysed by resuspending the cell pellet in 5 mL of ACK lysis buffer and incubating on ice for 5 min. The ACK lysis buffer was quenched with 10 mL of DMEM+10% FBS and the cells were pelleted and resuspended in 200 μL of PBS. The isolated meninges of three mice were pooled for each sample to obtain sufficient cells for flow cytometry analysis.

### Isolation of Femoral Bone Marrow

Mouse femurs were collected and aspirated with RPMI 1640 medium containing 10% fetal bovine serum (FBS). Red blood cells were lysed by resuspending the cell pellet in 5 mL of ACK lysis buffer and incubating on ice for 5 min. The ACK lysis buffer was quenched with 10 mL of DMEM+10% FBS and the cells were pelleted and resuspended in 200 μL of PBS.

### Cytospin of Meningeal Isolates

Glass 96-well plates were coated with 100 μL of poly-D-lysine (Sigma Aldrich, 0.1 mg mL⁻¹) in water at room temperature for 1 h and then washed three times with sterile PBS and allowed to air dry for 15 min in a tissue culture hood. Approximately 30,000 meningeal cells were placed in each well and the plate was spun down at 40 g for 10 min at 20° C. The cells were fixed with 4% paraformaldehyde for 15 min at room temperature, quenched with 10 mM NH₄Cl in PBS for 10 min at room temperature, permeabilized with 0.10% Triton X-100 in PBS for 10 min at room temperature, and blocked in 5% normal goat serum in PBS with 0.05% Tween-20 (PBS-T) for 10 min at room temperature. The cells were stained with rabbit anti-human/mouse GDNF antibody (Abcam, 2.5 μg mL⁻¹) at room temperature for 1 h. The wells were then washed and followed by incubation in donkey anti-rabbit IR 680D-conjugated secondary antibody (Abcam, 5 μg mL⁻¹) at room temperature for 2 h. After washing off the secondary, the cells were incubated in 1:5000 DAPI for 5 min at room temperature, washed, and then placed in PBS. The plate was immediately imaged on a Leica SP8 confocal microscope (Leica). ImageJ was used to quantify raw integrated pixel density per cell.

### Cₑ3D Optical Clearing and Tissue Immunofluorescence

Staining and tissue clearing were conducted as described by Li et al. (Li, W., Germain, R. N. & Gemer, M. Y. Multiplex, quantitative cellular analysis in large tissue volumes with clearing-enhanced 3D microscopy (Ce3D). Proceedings of the National Academy of Sciences 114, E7321-E7330, doi:10.1073/pnas.1708981114 (2017)) and Wang (Wang, Y. et al. Early developing B cells undergo negative selection by central nervous system-specific antigens in the meninges. Immunity 54, 2784-2794.e2786, doi:10.1016/j.immuni.2021.09.016 (2021))ran. Briefly, mice were injected with 2 nmol of Osteosense 680 (Perkin Elmer) via tail vein injection 24 h before euthanasia. Mice were cardiac perfused with cold PBS, calvaria were removed and fixed in PLP buffer at 4° C. for 24 h. Calvaria were washed 3×30 min with wash buffer (0.2% Triton X-100, 0.5% 1-thioglycerol in PBS) and then blocked (1% mouse serum, 1% BSA, 0.3% Triton X-100 in PBS) at 37° C. for 24 h at 200 rpm. Calvaria were incubated with AF488-conjugated mouse anti-mouse α-SMA (1.25 ug mL⁻¹) and AF568-conjugated anti-RFP (Rockland) (1.25 ug mL⁻¹) in blocking buffer at 37° for 24 h at 200 rpm. Calvaria were then washed for 14 h at 37° C. at 200 rpm and then for 24 h at 25° C. Calvaria were then cleared in Ce3D clearing solution (22% N-methylacetamide, 0.8 g mL⁻¹ histodenz, 0.1% Triton X-100, 0.5% 1-thioglycerol in PBS) for 72 h at room temperature. Calvaria were then mounted on a coverslip in a slide spacer in Ce3D solution and imaged on a Leica SP8 confocal microscope. The entire calvarium was scanned to search for tdT+ cells align emissary vessels.

### IVIS Bioluminescence Imaging

Mice were engrafted with 1833-Luciferase-Parental or -α6 KO cells via intracardiac engraftment for IVIS imaging on day 28 post-engraftment. Briefly, mice were administered 150 mg/kg body weight of Xenolight D-luficerin (Perkin Elmer) via intraperitoneal injection. After 15 minutes, anesthetized mice were imaged on an IVIS Kinetic Imager (Perkin Elmer) in the prone and supine position and radiance (photons) was collected. Living Image (Perkin Elmer) was used to quantify total flux (photons/s) In defined regions of interest.

### Antibodies for Flow Cytometry

The following antibodies were purchased from BD Biosciences: PerCP-Cy5.5-conjugated rat anti-human/mouse CD49f, BV421-conjugated rat anti-mouse CD56, FITC-conjugated rat anti-mouse CD3, FITC-conjugated rat-anti mouse CD19, AF700-conjugated rat anti-mouse CD45, APC-Cy7-conjugated rat anti-mouse CD11b, BV711-conjugated rat anti-mouse F4/80, BV605-conjugated rat-anti-mouse Ly-6C, BUV395-conjugated rat anti-mouse Ly-6G, PE-Cy7-conjugated rat anti-mouse CD45, APC-Cy7-conjugated rat anti-mouse CD3, AF700-conjugated rat-anti mouse CD4, PerCP-Cy5.5-conjugated rat anti-mouse CD8, BV711-conjugated hamster anti-mouse PD-1, BV421-conjugated mouse anti-mouse TIM-3, BV605-conjugated mouse anti-mouse Slamf6, AF647-conjugated rat anti-mouse Foxp3, BV650-conjugated rat anti-mouse LAG-3, AF488-conjugated anti-Ki-67, BV786-conjugated rat anti-mouse CD62L, BV786-conjugated rat anti-mouse CD19, BIV737-conjugated mouse anti-mouse NK-1.1. The following antibodies were purchased from BioLegend: PE-Cy7-conjugated rat anti-mouse CD49d, PE/Dazzle 594-conjugated rat anti-mouse CD44, PE/Dazzle 594-conjugated rat anti-mouse CD49f. Rabbit anti-human/mouse GDNF antibody was purchased from Thermo Fisher and conjugated with a PerCP-Cy5.5 conjugation kit from Abcam.

### Flow Cytometry

0.5×10⁶ cultured cells or 0.8×10⁵ meningeal cells were washed with autoMACS (Miltenyi)+3% BSA. Cells were blocked in 100 μL of ‘SuperBlock’ (2% FBS, 5% normal rat serum, 5% normal mouse serum, 5% normal rabbit serum and anti-mouse FcR (BD Biosciences, 10 g mL⁻¹) in PBS) for 20 min on ice. Cells were washed and resuspended in 100 μL of either Live/Dead Fixable Near-IR (Thermo Fisher) or Zombie UV Fixable Viability Kit (BD Biosciences) and incubated on ice for 20 min. Cells were washed and incubated in 100 μL of primary antibody on ice for 25 min. For intracellular stained samples, after surface staining cells were fixed and permeabilized in 250 μL of Cytofix/Cytoperm (BD Biosciences) for 30 min on ice and then washed and incubated with 50 μL of primary antibody diluted in 1× Perm/Wash buffer (BD Biosciences). After staining, cells were washed and resuspended in 300 μL of PBS and analyzed on a flow cytometer. Flow cytometry was conducted on a BD FACSCanto II or a BD LSRFortessa X-20 (BD Biosciences) at the Duke Cancer Institute Flow Cytometry Core. Cell sorting was conducted on a BD FACSAria II (BD Biosciences) at the Duke Human Vaccine Institute Flow Cytometry Core. Data was analyzed in FlowJo 10 (BD Biosciences).

### Western Blot Analysis

More than 1×10⁶ cultured cells were collected by centrifugation and washed with ice-cold PBS. Ice-cold RIPA buffer (50 mM Tris-HCl (pH 7.4), 0.25 M NaCl, 5 mM EDTA, 20 mM NaF, 1% NP-40) containing fresh protease inhibitor cocktail (Sigma Aldrich) and phosphatase inhibitor cocktails 2 and 3 (Sigma Aldrich) was added to the cells. The suspension was transferred into a centrifuge tube and placed on ice for 3 min. The cell suspension was cleared by centrifugation at 14,000 g for 3 min at 4° C. The supernatants (total cell lysate) were used immediately or stored at −80° C. Protein concentrations were determined using the DC Protein Assay (Bio-Rad Laboratories). Samples (20 μg of protein) were analyzed using the following primary antibodies, as indicated: anti-NCAM1 (Cell Signaling Technology), anti-NCAM2 (Cell Signaling Technology), anti-integrin α6 (Cell Signaling Technology), GAPDH (Cell Signaling Technology). Horseradish peroxidase (HRP)-coupled rabbit IgG (Cell Signaling Technology) was used as a secondary antibody, and immunoreactive proteins were detected by enhanced chemiluminescence (ECL) (ThermoFisher).

### RT-Qpcr

Cells were cultured in tissue culture treated flasks for 48 h to about 75% confluence and RNA was extracted using a Qiagen RNeasy kit (Qiagen). cDNA was prepared using Maxima First Strand cDNA Synthesis Kit for RT-qPCR (Thermo Fisher). TaqMan master mix and probes (Thermo Fisher) were used for the amplification of target and housekeeping genes on the ViiA 7 Real-Time PCR System (Thermo Fisher). The delta-delta Ct method was used for calculation of fold change.

### Three-Dimensional Invasion Assays

Cells were seeded for 16 h in media containing 0.3% FBS. Cells were resuspended in serum-free rat-tail collagen (Advanced Biomatrix, 3 mg mL⁻¹) supplemented with recombinant laminin 511 (Biolamina, 0.1 mg mL⁻¹). Resuspended cells were aliquoted into 96-well plates and spun down to the bottom of the plate. Collagen was allowed to polymerize for 1 h and cell culture medium containing 20% FBS was added on top of the gel as a chemoattractant. After 24 h of incubation at 37° C., plates were fixed and stained with Hoechst 33258 (Molecular Probes-Life Technologies, 5 g mL⁻¹). Plates were imaged on a Leica SP8 microscope. The three-dimensional migration index was calculated as number of invading cells at 50 μm divided by the total number of cells.

### Migration Assays

Optical bottom 96-well plates were coated with either recombinant laminin 511 (Biolamina, 0.1 mg mL⁻¹) or BSA (0.1 mg mL⁻¹) for 24 h at 4° C. 1,000 cells were seeded in each well in 100 μL of media and allowed to settle for 1 h prior to imaging. A 5×5 tile-scan image was collected in each well on a Leica SP8 confocal microscope with an incubator attachment every 15 min for 24 h using a 10× objective. Data were imported into Imaris and cells were tracked to calculate mean displacement over 24 h.

### GDNF Glucose Starvation Assays

1×10⁵ 1833 or EO-LM2 cells were seeded in glucose free or complete media+1% FBS in a 12-well plate. The cells were treated with recombinant human/mouse GDNF (PeproTech, 100 ng mL⁻¹) or vehicle (0.1% BSA in water) and anti-CD49f neutralizing antibody (Invitrogen, 2.5 μg mL⁻¹) or isotype control. The cells were incubated at 37° C. and 5% CO₂ for 24 h (EO-LM2) or 48 h (1833) and then trypsinized and counted with a hemacytometer. The number of live cells in the glucose deprived conditions were then divided by the number of cells in the glucose complete condition to determine the percent of cells alive after glucose deprivation.

### Proliferation Assays

0.75×10⁵ 1833/EO-LM2 or 0.5×10⁵ MCF7 cells were seeded in a 6-well plate in 2 mL of media. Every 24 h, cells were trypsinized, resuspended in 200 μL of media, and counted on a hemacytometer.

### Human Samples

Patients were recruited to our retrospective case-control study by searching the Duke University electronic medical records using the DEDUCE (Duke Enterprise Data Unified Content Explorer) on-line research portal. Diagnostic codes indicating breast cancer and CNS disease were used to identify an initial patient cohort and charts were reviewed to confirm the diagnoses. Breast cancer patients with CNS disease confirmed through tissue biopsy were retrospectively enrolled. Patients identified by our DEDUCE search, but for whom craniotomy biopsy tissue blocks were unavailable (e.g., the biopsy was performed at an outside hospital, the archived sample was misplaced) were excluded from our study. These occurrences could potentially create a sample selection bias/systematic selection error in our study if patients whose biopsies were performed at an outside hospital or whose samples were lost at Duke were not representative of the patient population as a whole. This is a common limitation of a case-control study; therefore, the results of this patient study are construed to support the overall findings of our manuscript but are not stated as providing definitive evidence of cause and effect in this patient population.

### Quantification of CD68/GDNF Dual Stained Biopsy Samples

Whole slide images were acquired with the GT450 imaging system (Leica Biosystems). Acquired images were loaded into the Visiopharm Oncotopix software (Visiopharm). Images were then processed using a machine learning algorithm using a decision forest pixel classifier as follows. Training regions were selected and hand annotated to distinguish GDNF staining (yellow), CD68 staining (magenta), and background. The algorithm was trained and tested in several patches in multiple slide images to ensure adequate model performance. For the final analysis an overall tissue detection step was run, and the classifier was run on the entire tissue area. Postprocessing was performed to remove small holes in GDNF areas and simplify contours of the GNDF and CD68 annotated areas with sequential dilation and erosion steps. The algorithm identified CD68 positive cells contacting GDNF positive areas and those not contacting GDNF. CD68 positive cells were counted and total area computed for both GNDF contacting and non-contacting cells. Additionally computed were total tissue area and total GDNF positive tissue area for each specimen. For manual quantification, slight image color adjustments were employed for each image to improve the contrast of cell nuclei from background. Subsequently, ten arbitrarily selected 40× fields were identified from low power (2×) to maximize representation of GDNF+ areas but at insufficient resolution to visualize CD68+ staining (WRJ). These images were then counted by a board certified hematopathologist (JN) to enumerate the number of CD68+ cells out of total cells within GDNF+ areas.

### Statistics and Reproducibility

Two-sided, unpaired Student's t-test was used for analysis of 3D invasion assay, migration assay, Ncam1 flow quantification validation, IVM quantification of EO-LM2 vs. −NCAM1 KO, and quantification of GDNF IF for vehicle vs. PLX5622 treated EO-LM2 mice. One-way ANOVA with Tukey's post-hoc test was used for analysis of bone channel diameters, IVM quantification on EO-P vs. −LM2 vs. −LM2-α6 KO, GDNF expression via flow cytometry, and glucose starvation assays. Two-way ANOVA with Tukey's post-hoc test was used for analysis of GDNF IF comparing healthy vs. diseased mice and for IVM quantification of MacGreen-engrafted mice treated with PLX5622 or vehicle. Kaplan-Meier curves with two-sided log rank Mantel-Cox analysis was used for in vivo survival studies. Fisher's exact test was used for analysis of HLP incidence and LMD incidence (MCF7 vs α6 OE). Chi-squared test was used for integrin α6 expression of patient data. Kruskal-Wallis with Dunn's multiple comparisons post-hoc test was used for analysis of HLP score enrichment in derivation of EO-LM2 cells. One-sample t-test against zero on log-transformed values was used to quantify intravascular vs. abluminal EO-LM2 cells on day 3. Statistical analysis was conducted in GraphPad Prism version 9.0. Significant P values were defined as follows: ns, non significant; *P<0.05; **P<0.01; ***P<0.001; ****P<0.0001. Data are mean s.e.m. Precise P values for data shown in the main figures using these ranges are as follows. FIG. 1d: P 0.9617 (Parental vs. LM1), ***P=0.0010 (Parental vs. EO-LM2). FIG. 1i: **P=0.0076. FIG. 1j: *P=0.0104, **P=0.0037. FIG. 7a: **P=0.0023. FIG. 7b: **P=0.0025. FIG. 7c: *P=0.0406. FIG. 7e: ****P<0.0001. FIG. 7f: ***P=0.0002. FIG. 7g: ***P =0.0002. FIG. 7i: *P=0.0152. FIG. 7k: *P=0.0242, **P=0.0088. FIG. 11b: ****P<0.0001. FIG. 11c: P=0.6318. FIG. 11d. *P=0.0476. FIG. 11e: EO-LM2: *P=0.0120 (vehicle/isotype vs. GDNF/isotype), * P=0.0277 (GDNF/isotype vs. GDNF/anti-NCAM), P =0.7475 (vehicle/isotype vs GDNF/anti-NCAM). 1833-P: *P=0.0203 (vehicle/isotype vs. GDNF/isotype), *P=0.0256 (GDNF/isotype vs. GDNF/anti-NCAM), P=0.9733 (vehicle/isotype vs. GDNF/anti-NCAM). FIG. 11f P=0.5324 (healthy CSF1R+vs. healthy CSF1R−), *P=0.0159 (healthy CSF1R− vs. LMD CSF1R−), ****P<0.0001 (healthy CSF1R+vs. LMD CSF1R+), ****P<0.0001 (LMD CSF1R+vs. LMD CSF1R−). FIG. 11i: ***P=0.0003. FIG. 3k. ***P=0.0002. FIG. 3l: *P=0.0232. FIG. 11m P>0.9999 (D3 co-loc. vehicle vs. PLX5622), P >0.9999 (D3 co-loc. PLX5622 vs D10 co-loc. PLX5622), ****P<0.0001 (D3 co-loc. vehicle vs. D10 co-loc. vehicle), ****P<0.0001 (D10 co-loc. vehicle vs. PLX5622). FIG. 11p: ***P=0.0002. FIG. 19a: ***P=0.0002.

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

### INCORPORATION BY REFERENCE

The entire disclosure of each of the patent documents and scientific articles referred to herein is incorporated by reference for all purposes. Complete citations for the references cited within the application are provided within the following reference list. Indeed, each of the following references are herein incorporated by reference in their entireties:
1 Franzoi, M. A. & Hortobagyi, G. N. Leptomeningeal carcinomatosis in patients with breast cancer. Critical Reviews in Oncology/Hematology 135, 85-94, doi:https://doi.org/10.1016/j.critrevonc.2019.01.020 (2019).
2 Gauthier, H. et al. Survival of breast cancer patients with meningeal carcinomatosis. Annals of Oncology 21, 2183-2187, doi:10.1093/annonc/mdq232 (2010).
3 Oechsle, K., Lange-Brock, V., Kruell, A., Bokemeyer, C. & de Wit, M. Prognostic factors and treatment options in patients with leptomeningeal metastases of different primary tumors: a retrospective analysis. Journal of Cancer Research and Clinical Oncology 136, 1729-1735, doi: 10.1007/s00432-010-0831-x (2010).
4 Wang, N., Bertalan, M. S. & Brastianos, P. K. Leptomeningeal metastasis from systemic cancer: Review and update on management. Cancer 124, 21-35 (2018).
5 Pellerino, A. et al. Management of Brain and Leptomeningeal Metastases from Breast Cancer. International Journal of Molecular Sciences 21, doi:10.3390/ijms21228534 (2020).
6 Derk, J., Jones, H. E., Como, C., Pawlikowski, B. & Siegenthaler, J. A. Living on the Edge of the CNS: Meninges Cell Diversity in Health and Disease. Frontiers in Cellular Neuroscience 15 (2021).
7 Mastorakos, P. & McGavern, D. The anatomy and immunology of vasculature in the central nervous system. Science Immunology 4, 1-15, doi:10.1126/sciimmunol.aav0492 (2019).
8 Boire, A. et al. Complement Component 3 Adapts the Cerebrospinal Fluid for Leptomeningeal Metastasis. Cell 168, 1101-1113, doi:10.1016/j.cell.2017.02.025 (2017).
9 Kokkoris, C. P. Leptomeningeal carcinomatosis: How does cancer reach the pia-arachnoid?Cancer 51, 154-160, doi:10.1002/1097-0142(19830101)51:1<154::AID-CNCR2820510130>3.0.CO; 2-K (1983).
10 Yao, H. et al. Leukaemia hijacks a neural mechanism to invade the central nervous system. Nature 560, 55-60, doi:10.1038/s41586-018-0342-5 (2018).
11 Brioschi, S. et al. Heterogeneity of meningeal B cells reveals a lymphopoietic niche at the CNS borders. Science 373, eabf9277, doi:10.1126/science.abf9277 (2021).
12 Cugurra, A. et al. Skull and vertebral bone marrow are myeloid cell reservoirs for the meninges and CNS parenchyma. Science 373, eabf7844, doi:10.1126/science.abf7844 (2021).
13 Cai, R. et al. Panoptic imaging of transparent mice reveals whole-body neuronal projections and skull-meninges connections. Nature Neuroscience 22, 317-327, doi:10.1038/s41593-018-0301-3 (2019).
14 Herisson, F. et al. Direct vascular channels connect skull bone marrow and the brain surface enabling myeloid cell migration. Nature Neuroscience 21, 1209-1217, doi:10.1038/s41593-018-0213-2 (2018).
15 Valiente, M. et al. Brain Metastasis Cell Lines Panel: A Public Resource of Organotropic Cell Lines. Cancer Research 80, 4314-4323, doi:10.1158/0008-5472.CAN-20-0291 (2020).
16 Dai, J. et al. Astrocytic laminin-211 drives disseminated breast tumor cell dormancy in brain. Nature Cancer 3, 25-42, doi:10.1038/s43018-021-00297-3 (2022).
17 Kienast, Y. et al. Real-time imaging reveals the single steps of brain metastasis formation. Nature Medicine 16, 116-122, doi:10.1038/nm.2072 (2010).
18 Li, W., Germain, R. N. & Gerner, M. Y. Multiplex, quantitative cellular analysis in large tissue volumes with clearing-enhanced 3D microscopy (Ce3D). Proceedings of the National Academy of Sciences of the United States of America 114, E7321-E7330 (2017).
19 Wang, Y. et al. Early developing B cells undergo negative selection by central nervous system-specific antigens in the meninges. Immunity 54, 2784-2794.e2786, doi:https://doi.org/10.1016/j.immuni.2021.09.016 (2021).
20 Nishiuchi, R. et al. Characterization of the Ligand-Binding Specificities of Integrin α3β1 and α6β1 Using a Panel of Purified Laminin Isoforms Containing Distinct a Chains. The Journal of Biochemistry 134, 497-504, doi:10.1093/jb/mvg185 (2003).
21 Nishiuchi, R. et al. Ligand-binding specificities of laminin-binding integrins: A comprehensive survey of laminin-integrin interactions using recombinant α3β1, α6β1, α7β1 and α6β4 integrins. Matrix Biology 25, 189-197, doi:https://doi.org/10.1016/j.matbio.2005.12.001 (2006).
22 Nielsen, J. et al. Role of Glial Cell Line-Derived Neurotrophic Factor (GDNF)-Neural Cell Adhesion Molecule (NCAM) Interactions in Induction of Neurite Outgrowth and Identification of a Binding Site for NCAM in the Heel Region of GDNF. The Journal of Neuroscience 29, 11360-11376, doi:10.1523/JNEUROSCI.3239-09.2009 (2009).
23 Paratcha, G., Ledda, F. & Ibáñez, C. F. The Neural Cell Adhesion Molecule NCAM Is an Alternative Signaling Receptor for GDNF Family Ligands. Cell 113, 867-879, doi:https://doi.org/10.1016/S0092-8674(03)00435-5 (2003).
24 Batchelor, P. E. et al. Activated Macrophages and Microglia Induce Dopaminergic Sprouting in the Injured Striatum and Express Brain-Derived Neurotrophic Factor and Glial Cell Line-Derived Neurotrophic Factor. The Journal of Neuroscience 19, 1708, doi:10.1523/JNEUROSCI.19-05-01708.1999 (1999).
25 Ortega-de San Luis, C. & Pascual, A. Simultaneous Detection of Both GDNF and GFRα1 Expression Patterns in the Mouse Central Nervous System. Frontiers in Neuroanatomy 10 (2016).
26 Batchelor, P. E. et al. Macrophages and Microglia Produce Local Trophic Gradients That Stimulate Axonal Sprouting Toward but Not beyond the Wound Edge. Molecular and Cellular Neuroscience 21, 436-453, doi:https://doi.org/10.1006/mcne.2002.1185 (2002).
27 Biju, K. C. et al. Macrophage-mediated GDNF Delivery Protects Against Dopaminergic Neurodegeneration: A Therapeutic Strategy for Parkinson's Disease. Molecular Therapy 18, 1536-1544, doi:https://doi.org/10.1038/mt.2010.107 (2010).
28 Chen, C. et al. GDNF-expressing macrophages mitigate loss of dopamine neurons and improve Parkinsonian symptoms in MitoPark mice. Scientific Reports 8, 5460, doi:10.1038/s41598-018-23795-4 (2018).
29 Ibáñez, C. F. & Andressoo, J.-O. Biology of GDNF and its receptors—Relevance for disorders of the central nervous system. Neurobiology of Disease 97, 80-89, doi:https://doi.org/10.1016/j.nbd.2016.01.021 (2017).
30 Spector, R., Robert Snodgrass, S. & Johanson, C. E. A balanced view of the cerebrospinal fluid composition and functions: Focus on adult humans. Experimental Neurology 273, 57-68, doi:https://doi.org/10.1016/j.expneurol.2015.07.027 (2015).
31 Spangenberg, E. et al. Sustained microglial depletion with CSF1R inhibitor impairs parenchymal plaque development in an Alzheimer's disease model. Nature Communications 10, 3758, doi:10.1038/s41467-019-11674-z (2019).
32 Valdearcos, M. et al. Microglia Dictate the Impact of Saturated Fat Consumption on Hypothalamic Inflammation and Neuronal Function. Cell Reports 9, 2124-2138, doi:https://doi.org/10.1016/j.celrep.2014.11.018 (2014).
33 Rua, R. & McGavem, D. B. Advances in Meningeal Immunity. Trends in Molecular Medicine 24, 542-559, doi: 10.1016/j.molmed.2018.04.003 (2018).
34 Chi, Y. et al. Cancer cells deploy lipocalin-2 to collect limiting iron in leptomeningeal metastasis. Science 369, 276-282, doi:10.1126/science.aaz2193 (2020).
35 Boire, A., Brastianos, P. K., Garzia, L. & Valiente, M. Brain metastasis. Nature Reviews Cancer 20, 4-11, doi:10.1038/s41568-019-0220-y (2020).
36 Martirosian, V. et al. Medulloblastoma uses GABA transaminase to survive in the cerebrospinal fluid microenvironment and promote leptomeningeal dissemination. Cell Reports 35, 109302, doi:https://doi.org/10.1016/j.celrep.2021.109302 (2021).
37 Flanagan, L. A., L. M., R., Derzic, S. & Monuki, E. S. Regulation of human neural precursor cells by laminin and integrins. J Neurosci Res 83, 845-856 (2006).
38 Cremer, H., Chazal, G., Goridis, C. & Represa, A. NCAM Is Essential for Axonal Growth and Fasciculation in the Hippocampus. Molecular and Cellular Neuroscience 8, 323-335, doi:https://doi.org/10.1006/mcne.1996.0588 (1997).
39 Duncan, B. W., Murphy, K. E. & Maness, P. F. Molecular Mechanisms of L1 and NCAM Adhesion Molecules in Synaptic Pruning, Plasticity, and Stabilization. Front Cell Dev Biol 9 (2021).
40 Bovetti, S. et al. Blood Vessels Form a Scaffold for Neuroblast Migration in the Adult Olfactory Bulb. Journal of Neuroscience 27, 5976-5980, doi:10.1523/jneurosci.0678-07.2007 (2007).
41 Bamhill, R. L. et al. Atypical Spitzoid Melanocytic Neoplasms With Angiotropism: A Potential Mechanism of Locoregional Involvement. The American Journal of Dermatopathology 33, 236-243 (2011).
42 Diestel, S., Hinkle, C., Schmitz, B. & Maness, P. F. NCAM140 stimulates integrin-dependent cell migration by ectodomain shedding. Journal of Neurochemistry 95, 1777-1784 (2005).
43 Frame, M. C. & Inman, G. J. NCAM Is at the Heart of Reciprocal Regulation of E-Cadherin- and Integrin-Mediated Adhesions via Signaling Modulation. Developmental Cell 15, 494-496, doi:https://doi.org/10.1016/j.devcel.2008.09.016 (2008).
44 Andersen, B. M. et al. Glial and myeloid heterogeneity in the brain tumour microenvironment. Nature Reviews Cancer 21, 786-802, doi:10.1038/s41568-021-00397-3 (2021).
45 Guldner, I. H. et al. CNS-Native Myeloid Cells Drive Immune Suppression in the Brain Metastatic Niche through Cxcl10. Cell 183, 1234-1248, doi:https://doi.org/10.1016/j.cell.2020.09.064 (2020).
46 Bowman, R. L. et al. Macrophage Ontogeny Underlies Differences in Tumor-Specific Education in Brain Malignancies. Cell Reports 17, 2445-2459, doi:10.1016/j.celrep.2016.10.052 (2016).
47 Klemm, F. et al. Interrogation of the Microenvironmental Landscape in Brain Tumors Reveals Disease-Specific Alterations of Immune Cells. Cell 181, 1643-1660.e1617, doi:https://doi.org/10.1016/j.cell.2020.05.007 (2020).
48 Schulz, M. et al. Cellular and Molecular Changes of Brain Metastases-Associated Myeloid Cells during Disease Progression and Therapeutic Response. iScience 23, 101178, doi:https://doi.org/10.1016/j.isci.2020.101178 (2020).
49 Smalley, I. et al. Single-Cell Characterization of the Immune Microenvironment of Melanoma Brain and Leptomeningeal Metastases. Clinical Cancer Research 27, 4109-4125, doi:10.1158/1078-0432.CCR-21-1694 (2021).
50 Munro David, A. D., Movahedi, K. & Priller, J. Macrophage compartmentalization in the brain and cerebrospinal fluid system. Science Immunology 7, eabk0391, doi:10.1126/sciimmunol.abk0391 (2022).

What is claimed is:

1. A method for determining if a subject is suffering from leptomeningeal disease (LMD) comprising obtaining a biological sample from the subject, detecting the presence or absence of one or more of: integrin α6, BCCs expressing integrin α6, NCAM, GDNF, NRTN, ARTN, and PSPN in the biological sample, and determining that the subject is suffering from or at risk of suffering from LMD if one or more of: integrin α6, BCCs expressing integrin α6, NCAM, GDNF, NRTN, ARTN, and PSPN are detected in the biological sample.

2. The method of claim 1, wherein the subject is a human subject suffering from breast cancer.

3. The method of claim 1,
wherein detecting the presence or absence of one or more of NCAM comprises detecting the presence or absence of NCAM signaling,
wherein detecting the presence or absence of one or more of GDNF comprises detecting the presence or absence of GDNF signaling,
wherein detecting the presence or absence of one or more of NRTN comprises detecting the presence or absence of NRTN signaling,
wherein detecting the presence or absence of one or more of ARTN comprises detecting the presence or absence of ARTN signaling,
wherein detecting the presence or absence of one or more of PSPN comprises detecting the presence or absence of PSPN signaling.

4. The method of claim 1, wherein the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

5. A method for predicting a risk of developing LMD, the method comprising providing a biological from a subject, determining the level of one or more of integrin α6, BCCs expressing integrin 06, GDNF, NRTN, ARTN, and PSPN in the biological sample; and comparing the determined level to a reference level; wherein a higher level of one or more of integrin α6, BCCs expressing integrin α6, GDNF, NRTN, ARTN, and PSPN in the biological sample compared to the reference level is indicative of an increased risk of developing LMD; and wherein a level equal to or lower than the reference level is not indicative of an increased risk of developing LMD.

6. The method of claim 5, wherein the reference level is an expression and/or activity level of one or more of integrin α6, BCCs expressing integrin α6, GDNF, NRTN, ARTN, and PSPN correlated with a low risk or no risk of developing LMD.

7. The method of claim 5, wherein the subject is a human subject suffering from breast cancer.

8. The method of claim 5, wherein the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

9. A method for characterizing a biological sample, comprising obtaining a biological sample from a subject, detecting the presence or absence of one or more of integrin α6, BCCs expressing integrin α6, NCAM, GDNF, NRTN, ARTN, and PSPN in the biological sample, and characterizing the biological sample as consistent with LMD if one or more of integrin α6, BCCs expressing integrin α6, NCAM, GDNF, NRTN, ARTN, and PSPN is detected in the biological sample.

10. The method of claim 9,
wherein detecting the presence or absence of one or more of NCAM comprises detecting the presence or absence of NCAM signaling,
wherein detecting the presence or absence of one or more of GDNF comprises detecting the presence or absence of GDNF signaling,
wherein detecting the presence or absence of one or more of NRTN comprises detecting the presence or absence of NRTN signaling,
wherein detecting the presence or absence of one or more of ARTN comprises detecting the presence or absence of ARTN signaling,
wherein detecting the presence or absence of one or more of PSPN comprises detecting the presence or absence of PSPN signaling.

11. The method of claim 9, wherein the subject is a human subject suffering from breast cancer.

12. The method of claim 9, wherein the biological sample is selected from breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

13. A method for treating LMD, preventing LMD, and/or ameliorating the symptoms of LMD in a subject in need thereof, the method comprising administering to the subject a therapeutically effective amount of an agent capable of one or more of the following: inhibiting expression of integrin α6, inhibiting activity of integrin α6, inhibiting BCC expression of integrin α6, inhibiting activity of BCCs expressing integrin α6, preventing metastasis of BCCs expressing integrin α6 to LM cells, preventing abluminal vascular LM invasion by BCCs expressing integrin α6, preventing BCCs expressing integrin α6 from entering the CNS along emissary vessels bridging bone marrow (BM) and LM, preventing survival of BCCs expressing integrin α6 in the LM, inhibiting LM related NCAM expression and/or activity, inhibiting LM related GDNF expression and/or activity, inhibiting LM related NRTN expression and/or activity, inhibiting LM related ARTN expression and/or activity, inhibiting LM related PSPN expression and/or activity, inhibiting LM related NCAM signaling, inhibiting LM related GDNF signaling, inhibiting LM related NRTN signaling, inhibiting LM related ARTN signaling, inhibiting LM related PSPN signaling, preventing engagement of BCCs having integrin α6 expression with vascular basement membrane laminin in LM cells, preventing encasement of BCC having integrin α6 expression in perivascular CSF1R+ meningeal macrophages, and preventing BCC having integrin α6 expression inducement of CSF1R+ cells to upregulate expression of the neuroprotective factor GDNF.

14. The method of claim 13, wherein the LMD is leptomeningeal, disseminated, and/or multicentric disease.

15. The method of claim 13, wherein the LMD is associated with one or more primary CNS tumors.

16. The method of claim 13, wherein the LMD is associated with one or more low-grade gliomas (LGGs).

17. The method of claim 13, wherein the symptoms of LMD are one or more of the following: headaches (usually associated with nausea, vomiting, light-headedness), gait difficulties from weakness or ataxia, memory problems, incontinence, sensory abnormalities, pain, and seizures.

18. The method of claim 13, wherein the symptoms of LMD are one or more of the following: cerebral involvement including headache, lethargy, papilledema, behavioral changes, and gait disturbance; cranial-nerve involvement including impaired vision, diplopia, hearing loss, and sensory deficits, including vertigo; and cranial-nerve palsies; and spinal-root involvement including nuchal rigidity and neck and back pain, or invasion of the spinal roots.

19. The method of claim 13, wherein the agent is a small molecule, an antibody, nucleic acid molecule (e.g., siRNA, antisense oligonucleotide), a mimetic peptide, or a recombinant peptide.

20. The method of claim 13,
wherein the agent is a small molecule or antibody capable of inhibiting activity and/or expression of BCCs expressing integrin α6β1; and/or
wherein the agent is a small molecule or antibody capable of inhibiting activity and/or expression of integrin α6β1.

21. A method for treating LMD, preventing LMD, and/or ameliorating the symptoms of LMD in a subject, the method comprising:
(a) determining the presence of absence of one or more of integrin α6, BCCs expressing integrin α6, NCAM, GDNF, NRTN, ARTN, and PSPN in a biological sample taken from the subject; and
(b) administering a therapeutically effective amount of an agent to the subject if one or more of integrin α6, BCCs expressing integrin α6, NCAM, GDNF, NRTN, ARTN, and PSPN is identified, wherein the agent is capable of one or more of the following: inhibiting expression of integrin α6, inhibiting activity of integrin α6, inhibiting BCC expression of integrin α6, inhibiting activity of BCCs expressing integrin α6, preventing metastasis of BCCs expressing integrin α6 to LM cells, preventing abluminal vascular LM invasion by BCCs expressing integrin α6, preventing BCCs expressing integrin α6 from entering the CNS along emissary vessels bridging bone marrow (BM) and LM, preventing survival of BCCs expressing integrin α6 in the LM, inhibiting LM related NCAM expression and/or activity, inhibiting LM related GDNF expression and/or activity, inhibiting LM related NRTN expression and/or activity, inhibiting LM related ARTN expression and/or activity, inhibiting LM related PSPN expression and/or activity, inhibiting LM related NCAM signaling, inhibiting LM related GDNF signaling, inhibiting LM related NRTN signaling, inhibiting LM related ARTN signaling, inhibiting LM related PSPN signaling, preventing engagement of BCCs having integrin α6 expression with vascular basement membrane laminin in LM cells, preventing encasement of BCC having integrin α6 expression in perivascular CSF1R+ meningeal macrophages, and preventing BCC having integrin α6 expression inducement of CSF1R+ cells to upregulate expression of the neuroprotective factor GDNF.

22. The method of claim 21, wherein the subject is a human subject suffering from breast cancer.

23. The method of claim 21, wherein the biological sample is selected from a breast tissue sample, a breast tumor sample, a breast cancer cell sample, a bone-marrow sample, a bone marrow tumor sample, a bone marrow cell sample, a brain tissue sample, a brain tumor sample, a brain cell sample, a brain cancer cell sample, a LM tissue sample, a LM cell sample, and a CSF sample.

24. The method of claim 21, wherein the LMD is leptomeningeal, disseminated, and/or multicentric disease.

25. The method of claim 21, wherein the LMD is associated with one or more primary CNS tumors.

26. The method of claim 21, wherein the LMD is associated with one or more low-grade gliomas (LGGs).

27. The method of claim 21, wherein the symptoms of LMD are one or more of the following: headaches (usually associated with nausea, vomiting, light-headedness), gait difficulties from weakness or ataxia, memory problems, incontinence, sensory abnormalities, pain, and seizures.

28. The method of claim 21, wherein the symptoms of LMD are one or more of the following: cerebral involvement including headache, lethargy, papilledema, behavioral changes, and gait disturbance; cranial-nerve involvement including impaired vision, diplopia, hearing loss, and sensory deficits, including vertigo; and cranial-nerve palsies; and spinal-root involvement including nuchal rigidity and neck and back pain, or invasion of the spinal roots.

29. The method of claim 21, wherein the agent is a small molecule, an antibody, nucleic acid molecule (e.g., siRNA, antisense oligonucleotide), a mimetic peptide, or a recombinant peptide.

30. The method of claim 21,
wherein the agent is a small molecule or antibody capable of inhibiting activity and/or expression of BCCs expressing integrin α6$1; and/or
wherein the agent is a small molecule or antibody capable of inhibiting activity and/or expression of integrin α6β1.

31. The method of claim 13 or 21, further comprising co-administering to the subject an additional therapeutic agent.

32. The method of claim 31, wherein the additional therapeutic agent is an anti-cancer therapy.

33. The method of claim 32, wherein the anti-cancer therapy comprises radiation therapy and/or a chemotherapeutic agent.

34. The method of claim 33, wherein the chemotherapeutic agent is one or more of the following: a taxane, a platinum-based agent, an anthracycline, an anthraquinone, an alkylating agent, a HER2 targeting therapy, vinorelbine, a nucleoside analog, ixabepilone, eribulin, cytarabine, a hormonal therapy, capecitabine, lapatinib, 5-FU, vincristine, etoposide, or methotrexate.

35. A method of preventing and/or treating metastatic cancer in a subject, the method comprising administering to the subject a therapeutically effective amount of an inhibitory molecule, or a pharmaceutical composition thereof, capable of inhibiting and/or downregulating the binding, signaling, and/or function of a molecule selected from the group consisting of integrin α6, GDNF, NCAM, GDNF/NCAM complex, macrophages and/or microglia, and combinations thereof.

36. A method of preventing and/or treating systemic and brain parenchymal metastases in a subject, the method comprising: administering to the subject a therapeutically effective amount of an inhibitory molecule, or a pharmaceutical composition thereof, capable of inhibiting and/or downregulating the binding, signaling, and/or function of a molecule selected from the group consisting of: integrin α6, GDNF, NCAM, GDNF/NCAM complex, macrophages and/or microglia, and combinations thereof such that the systemic and brain parenchymal metastases is prevented and/or treated.

37. A method of preventing and/or treating leptomeningeal disease in a subject, the method comprising: administering to the subject a therapeutically effective amount of an inhibitory molecule, or a pharmaceutical composition thereof, capable of inhibiting and/or downregulating the binding, signaling, and/or function of a molecule selected from the group consisting of integrin α6, GDNF, NCAM, GDNF/NCAM complex, macrophages and/or microglia, and combinations thereof such that the leptomeningeal disease is prevented and/or treated.

38. The method as in any of claims 35-36 or 37 in which the inhibitory molecule is selected from the group consisting of an antibody, oligonucleotide, aptamer, small inhibitory molecule, and combinations thereof.

39. The method according to claim 38 in which the inhibitory molecule comprises an antibody.

40. The method according to claim 39 in which the antibody is specific to α6β1.

41. The method according to claim 39 in which the antibody is specific to GDNF.

42. The method according to claim 39 in which the antibody is specific to NCAM.

43. The method according to claim 39 in which the antibody is capable of inhibiting and/or disrupting the GDNF/NCAM complex.

44. The method according to claim 38 in which the inhibitory molecule comprises a small inhibitory molecule.

45. The method according to claim 44 in which the small inhibitory molecule is capable of inhibiting and/or disrupting the GDNF/NCAM complex and/or signaling pathway.
